(19) United States
(12) Patent Application Publication
(10) Pub. No.: US 2015/0037297 A1
(43) Pub. Date: Feb. 5, 2015

(54) Sickled Erythrocytes and Progenitors Target Cytotoxics to Tumors

(71) Applicant: David S Terman, Pebble Beach, CA (US)

(72) Inventor: David S Terman, Pebble Beach, CA (US)

(21) Appl. No.: 14/222,292

(22) Filed: Mar. 21, 2014

Related U.S. Application Data
(63) Continuation-in-part of application No. 13/367,797, filed on Feb. 7, 2012, now Pat. No. 8,431,117.
Application No. 13/367,797 is a continuation-in-part of application No. 14/037,176, filed on Sep. 25, 2013.
Application No. 14/037,176 is a continuation-in-part of application No. 13/317,590, filed on Oct. 20, 2011, now abandoned.
Application No. 13/317,590 is a continuation-in-part of application No. 12/586,532, filed on Sep. 22, 2009, now abandoned.
Application No. 12/586,532 is a continuation-in-part of application No. 13/328,748, filed on Dec. 16, 2011.
Application No. 13/328,748 is a continuation-in-part of application No. 13/317,590, filed on Oct. 20, 2011, now abandoned.
(60) Provisional application No. 61/455,592, filed on Oct. 20, 2010.

(51) Int. Cl. A61K 35/18

(52) U.S. Cl. CPC A61K 35/18; A61K 2035/124
USPC 424/93.21

(57) ABSTRACT

The present invention provides therapeutic mammalian cells which synthesize and express SS hemoglobin and a tumoricidal transgene. They are produced by transduction of SS erythroid progenitors/erythroblasts using viral vectors comprising a tumoricidal transgene operatively linked to the coding region of SS β-globin promoter/enhancer. Such transduced SS erythroid cells differentiate into mature SSRBCs that exhibit sustained synthesis and expression of SS hemoglobin, a tumoricidal protein(s). Both mature and progenitor SS-cells carrying tumoricidal transgene(s) are capable of selectively localizing in tumor microenvironment, occluding tumor microvessels and inducing a tumoricidal response.

### CROSS REFERENCE TO RELATED DOCUMENTS

The present application is a continuation in part of U.S. patent application Ser. No. 13/367,797 filed Feb. 7, 2012 which is a continuation in part of Ser. No. 12/586,532 filed Sep. 22, 2009 (abandoned) which is a continuation in part of U.S. patent application Ser. No. 12/276,941 filed Nov. 24, 2008, which is a continuation in part of Ser. No. 12/145,949 filed Jun. 25, 2008 (abandoned) which issued as U.S. Pat. No. 7,803,637 on Sep. 28, 2010 which is a divisional of U.S. patent application Ser. No. 10/937,758 filed Sep. 8, 2004 (abandoned) which is a continuation of U.S. patent application Ser. No. 09/650,884 filed Aug. 30, 2000 (abandoned) which is a continuation of U.S. provisional patent application 60/151,470 filed Aug. 30, 1999 (abandoned). All of the above patents and patent applications and their references are incorporated by reference in their entirety.

The present application is also a continuation in part of United States patent application Ser. No. 14/037,176 filed on Sep. 25, 2013 which is a continuation in part of U.S. patent application Ser. No. 13/317,590 filed Oct. 20, 2011 which claims priority to provisional U.S. patent application 61/455,592 filed Oct. 20, 2010 (abandoned). Both of these applications are incorporated by reference.

The present application is also a continuation in part of U.S. patent application Ser. No. 13/328,748 filed Dec. 16, 2011 which is a continuation in part of U.S. patent application Ser. No. 13/317,590 filed Oct. 20, 2011 which is a continuation in part of U.S. provisional application Ser. No. 61/455,592 filed Oct. 20, 2010 which is a continuation in part of U.S. patent application Ser. No. 12/586,532 filed Sep. 22, 2009. All or these patents and patent applications are incorporated in entirety by reference with their references. The present application claims priority U.S. provisional application 61/807,457 filed Apr. 2, 2013, U.S. patent application Ser. No. 13/367,797 filed Feb. 7, 2012, U.S. patent application Ser. No. 12/586,532 filed Sep. 22, 2009 (abandoned) and also claims priority to U.S. provisional application Ser. No. 61/215,906 filed May 11, 2009 (abandoned) and U.S. provisional application Ser. No. 61/211,227 filed Mar. 28, 2009 (abandoned) and U.S. provisional application Ser. No. 61/206,338 filed on Jan. 28, 2009 (abandoned) and U.S. provisional application Ser. No. 61/205,776 filed Jan. 22, 2009 (abandoned) and U.S. provisional application Ser. No. 61/192,949 filed on Sep. 22, 2008 (abandoned). All of the above patents and patent applications and their references are incorporated by reference in their entirety.

The following applications are related and incorporated by reference: PCT/US07/69869 filed May 29, 2007 (abandoned) and U.S. provisional application Ser. No. 60/809,553 filed on May 30, 2006 (abandoned) and U.S. provisional application Ser. No. 60/819,551 filed on Jul. 8, 2006 (abandoned) and U.S. provisional application Ser. No. 60/842,213 filed on Sep. 5, 2006 (abandoned) and U.S. patent application Ser. No. 10/428,817, filed May 5, 2003 (abandoned) and U.S. provisional application Ser. No. 60/438,686, filed Jan. 9^{th}, 2003 (abandoned) and U.S. provisional application Ser. No. 60/415,310, filed on Oct. 1^{st}, 2002 (abandoned) and U.S. provisional application Ser. No. 60/406,750, filed on Aug. 29, 2002 (abandoned) and U.S. provisional application Ser. No. 60/415,400, filed on Oct. 2^{nd}, 2002 (abandoned) and U.S. provisional application Ser. No. 60/406,697, filed on Aug. 28^{th}, 2002 (abandoned) and U.S. provisional application Ser. No. 60/389,366, filed on Jun. 15^{th}, 2002 (abandoned) and U.S. provisional application Ser. No. 60/378,988, filed on May 8, 2002 (abandoned) and U.S. patent application Ser. No. 09/870,759 filed on May 30, 2001 (abandoned) and U.S. patent application Ser. No. 09/650,884 filed Aug. 30, 2000 (abandoned) and U.S. provisional patent application Ser. No. 60/151,470 filed on Aug. 30, 1999 (abandoned).

### FIELD OF THE INVENTION

The invention is in the fields of genetics and medicine and covers compositions and methods for targeted delivery of anti-tumor agents using sickled erythrocytes, their nucleated precursors, and erythroleukemia cells in native state or upregulated for expression of constitutive adhesion molecules and transduced or loaded with hypoxia responsive elements, tumoricidal proteins, toxins, superantigens, hemolysins, oncolytic viruses, chemotherapeutics and anaerobic spores.

### BACKGROUND

Tumoricidal transgenes/proteins, oncolytic viruses/viral genomes and anti-tumor RNA or DNA nucleotides (collectively TTORs) delivered directly into the circulation face impediments to their localization to, and infection of, primary or metastatic tumors from seroreactive neutralizing antibodies or degradation by circulating nucleases. These obstacles to systemic delivery of TTORs are overcome by cellular vehicles, which afford their cargos protection in addition to tumor localization. Proteins and viruses can be loaded into, or onto, various cell vehicles without losing the biological activity of the virus, protein or cell carrier. Cellular vehicles that exhibit tropism for tumor cells or the tumor microenvironment are particularly efficient at ferrying tumoricidal molecules directly to primary and metastatic tumor even in the presence of neutralizing antibodies or circulating nucleases.

The present invention constitutes sickle red blood cells (SSRBCs) their nucleated precursors and sickle hemoglobin variants as vehicles for carriage of TTORs to tumors. Such SSRBCs and SS erythroid progenitor cells (SSEPCs) used for this purpose are a significant improvement over non-cell based submicron particles that deliver drug through blood circulation and subsequent extravasation to the tumor site. Despite development of numerous nanoparticulate drug carriers the therapeutic results have not met expectations or attained their theoretical advantages over use of free drug. As currently employed nanoparticles are limited in treating advanced disseminated and metastatic disease, because they require direct application of energy to the carriers within each individual tumor. For this reason, those methods are only effective for tumors at a tissue depth within reach of an applied energy field of sufficient strength to trigger release.

In further contrast to nanoparticles SSRBCs do not rely on the tumor targeted antibodies or tumor specific signaling molecules to guide them to the tumor. Instead, tumor localization of SSRBCs and SSEPCs relies on their affinity on specific disruptions of the normal tissue microenvironment induced by the tumor such as structurally chaotic vasculature and severe hypoxia (pO₂ levels ranging from 1-10%.). Such hypoxic zones are present in nearly all solid tumors and constitute a major cause of treatment resistance and tumor recurrence. In this hypoxic tumor microenvironment, SS hemoglobin polymerizes resulting in formation of membrane spicules and upregulation of a panoply or adhesion molecules. Such molecules BCAM/Lu, ICAM-4 and α4β1, adhere abnormally to cognate ligands laminin-α5, ανβ3, and VCAM-1 which are overexpressed on tumor endothelium. Such adhesion is amplified over time resulting in the formation of microaggregates that obstruct/occlude tumor vessels. SSRBCs trapped on the tumor endothelium produce superoxide/peroxide-driven hydroxyl radicals leading to membrane peroxidation and hemolysis. Hemoglobin released from hemolyzed SS cells is rapidly oxidized from ferro- to ferri-hemoglobin (methemoglobin) generating highly lipophilic heme-nitrosyl complexes that readily intercalate into cell membranes. Intracellular heme and its oxidative product free iron are highly toxic to cells catalyzing the oxidation of membrane lipids and DNA and activating caspases and cathepsins leading to perturbations of cytoskeleton and apoptosis. In this fashion, the SSRBCs by themselves are able to potentiate the tumoricidal effect of exogenously administered pro-oxidants (Terman D S et al., PLoS ONE 8(1): e52543. doi:10.1371 (2013)

Additional non-cell based drug carriers such as liposomes currently approved for clinical use have yet to surmount the problems of disintegration in the bloodstream, uptake by macrophages and Kupffer cells in liver and spleen and inablilty to pass through the endothelial cell barrier. Pegylated liposomes show reduced uptake by macrophages and a prolonged half-life but still have not exhibited sufficient localization to tumor tissues. Stealth liposomes in which a targeting molecule is attached to a pegylated residue have shown localization to tumors in vivo but to date, no significant therapeutic effects. Fusigenic molecules that promote fusion with the cell membrane, penetratin and TAT-mediated translocation, receptor mediated endocytosis have been employed to promote particle transfer across cell membranes but to date have produced no convincing anti-tumor effects. These constraints coupled with lackluster clinical results with nanoparticles and liposome call for conceptually new approaches for drug carriers (I. K. Kwon, et al., J. Control. Release 164: 108-114 (2012)).

In contrast, SSRBCs or SSEPCs possess a large carrying capacity and serve as excellent vehicles for transport of tumoricidal drugs, proteins and TORRs into the tumor mileau. SSEPCs transduced with tumoricidal transgenes whose encoded proteins, viruses and RNA nucleotides can be expressed and secreted by both the SSEPCs and SSRBCs. Expulsion of tumoricidal cargo from nucleated SS progenitor cells is effected by the unique adhesion of SSEPCs and SSRBCs to cognate receptors overexpresson on tumor endothelial cells. Such an adhesive synapse initiates signals that mobilize the transport of vesicles loaded with tumoricidal cargo to the intercellular adhesive synapse. Thereupon, they are transferred and endocytosed by target tumor endothelial cells. In mature SSRBCs, devoid of secretory vesicle machinery release of encapsulated drug payload into the tumor is accomplished by spontaneous autohemolysis of SSRBCs trapped on the tumor endothelium. Such autohemolysis in vivo is also induced by pretreatment of the cells with photo-oxidation which synchronizes the timing of in vivo hemolysis to coincide with maximum cellular accumulation in the tumor (Choe, Terman et al., J. Control. Release 171 184-192 (2013)).

The SSRBCs and SSEPCs also offer advantages over normal RBCs (nRBCs) cells as vehicles for carriage of oncolytic agents into tumors. While several anti-cancer agents have been encapsulated in nRBCs and used as drug carriers in vivo, they are rapidly engulfed and eliminated by the reticuloendothelial system. Their failure to accumulate in tumors and induce tumor killing severely limits the ability of nRBCs to selectively deliver a drug payload to tumors (Choe, Terman et al., supra (2013)).

T cells are used for adoptive immunotherapy but their preparation is arduous, expensive and problematic. Moreover, their specificity for tumor cells is diluted by their receptors for other tissues and organs such that less than 10% of the infused cells actually access the tumor in vivo. To obviate this concern, T cells are genetically transduced with single chain tumor specific fv fragment that recognize the tumor and provide costimulatory signals for cell activation. Such T cell stimulation, however, results in generation of toxicity-inducing cytokines such TNFα, IL-2 or IFNγ which can induce cytokine storm, shock, multiorgan failure and death. SSRBCs and SSEPCs do not produce significant amounts of such molecules after stimulation and thus avert their attendant toxicity. Whereas T cells must be obtained from blood or tumor of the same patient in order to avoid graft versus host disease, mature SSRBCs with blood type O, Rh⁻, antigens can be used as universal donors for treatment of all patients with cancer. Such SSRBCs can be obtained from induced pluripotent stem cells (SSiPPSCs) or embryonic stem cells (SSESCs) from sickle cell donors. Collectively, these drawbacks severely restrict clinical translation with T cells to a few centers with expertise in the preparation of these cell populations.

Cytokine induced T cells (CITS) are easier to prepare than T cells but suffer from the same considerations. Other cells vehicles such as mesenchymal stem cells are largely sequestered in the lungs after infusion and those cells that target the tumor are largely confined to the tumor stroma rather than the tumor cells or tumor endothelial cells. Embryonic progenitor endothelial cells target the tumor endothelium but the nature and phenotype of the cell population that actually does this has not been identified thus precluding consistent results with this carrier. Thus, SSRBCs and SSEPCs have highly novel tumor localizing properties and the ability to kill tumors directly or by carriage of tumoricidal transgenes and drugs into the tumor mileau. They offer advantages over submicron particles of spontaneously unloading their cargo in tumors without requiring an external energy source. Relative to other cellular carriers, they are universally available to all patients with cancer not just autologous donors and do not display graft versus host disease or the cytokine-induced toxicity associated with other T cell carriers.

The instant invention aims to promote the tumoricidal ability of SSEPCs and their differentiative progeny, mature SSRBCs, by arming them with tumoricidal transgenes, oncolytic viruses and regulatory RNAs. To accomplish this we introduce a unique lenitviral/HS2-4 β-globin expression vector in which the β globin coding region is replaced by a tumoricidal transgene. Transduction of SSEPCs with this vector initiates a surge in globin gene transcription resulting in robust activation and expression of a tumoricidal transgene together with endogenous sickle globin genes. Erythroid-specific expression of the transgene is driven by the β-globin promoter, two β-globin proximal enhancer sequences, and by the powerful Locus Control Region DNaseI hypersensitive regions (HS) 2-4.

This β-globin vector can accommodate and express tumoricidal transgenes encoding proteins or nucleic acids from eukaryotic and prokaryotic sources. Oncolytic viruses/genomes and regulatory RNA sequences are also integrated and expressed in this vector. By including an erythroid-specific transcriptional signal in the vector along with a tumoricidal transgene, the present invention exploits the genetic programming of the SSRBC which devotes almost its entire synthetic capabilities to the production of SS hemoglobin. SS progenitors transduced with this vector not only produce the transgene product but also differentiate into mature SSRBCs which continue to synthesize the tumoricidal molecule even after their nuclei have been extruded. To this end, we have now cloned a tumoricidal transgene (staphylococcal enterotoxin superantigen (SE)) into the β-globin coding region of this vector and demonstrated that it can efficiently transduce Sca-1+ bone marrow SS hematopoietic progenitor cells (SSHPCs) Importantly, upon reconstitution of lethally irradiated hosts with these transduced SS progenitor cells, the SE transgene product and SS hemoglobin are present at high levels in mature SSRBCs in the peripheral blood.

Consequently, by redirecting the red cell transcriptional signals toward inducing expression of a tumoricidal transgene, the present invention exploits the genetic programming of erythroid cells and renders these cells capable of synthesizing tumoricidal proteins and nucleotides. Thus, the instant invention, coupled with the inherent tumor targeting ability of SSEPCs and SSRBCs provides a hitherto unrecognized and efficient method for producing and delivering large amounts of tumoricidal proteins to hypoxic tumor niches notable for their recurrence and resistance to existing treatment.

### LEGEND TO FIGURE

FIG. 1. Insertion of a potent tumoricidal transgene into a lentiviral vector. Genes are illustrated by filled boxes and are represented according to scale. The lentiviral/tumoricidal transgene expression construct is shown. HS2 (1203 bp), HS3 (1213 bp), and HS4 (954 bp) sequences, the 3′ globin enhancer, the 266-βp-globin promoter (p) and the β-globin gene whose coding region is replaced by the tumoricidal transgene. The HIV-1 LTR is shown with a 3′ SIN deletion; indicates packaging signal; SD and SA, splice donor and acceptor sites, respectively; RRE, Rev-responsive element; cPPT/CTS, central polypurine tract or DNA flap/central termination sequence; and WPRE, woodchuck hepatitis virus post-transcriptional regulatory element.

### SUMMARY OF INVENTION

The present invention provides mature SS cells, SS progenitors or sickle hemoglobin variants that deposit in tumor vasculature undergo hemolysis releasing hemoglobin and heme. The latter are toxic to adjacent tumor endothelium and tumor cells and produce a tumoricidal response. The present invention also provides erythrocytes with SS hemoglobin, their nucleated precursors, sickle hemoglobin variants for targeted delivery of tumoricidal agents specifically to the microvasculature of the tumors. Selective generation of tumoricidal agents is effected by transduction of SS nucleated erythrocyte precursors with the a lentiviral or other vector in which the β-globin coding region is replaced by a transgene encoding a tumoricidal molecule (s). Such molecules include granzymes and perforin alone or operably fused to a beta adrenergic receptor. The latter construct is activated in mature sickle cells by catacholamines secreted by neuroblastomas, pheochromocytomas and medullocytomas. Transgenes encoding Staphylococcal enterotoxins SEG and SEI and pseudomonas exotoxins free or fused to tumor specific monoclonal antibodies or receptors are also useful for insertion into the lentiviral vector. Full or partial length oncolytic viral sequences or genomes are loaded into the β-globin coding region allowing full transcription of an oncolytic virus. Indeed, and regulatory RNAs are also loaded into the β-globin coding region any other tumoricidal transgene functional in this or other vectors is useful. The tumoricidal transgenes are under transcriptional control of the locus control region containing erythroid specific DNAase hypersensitivity regions, the β-globin promoter/enhancer found in the second intron of the β-globin gene as well as part of the third exon of β-globin and the enhancer located 3′ to the human β-globin gene, a poly A sequence and a β-globin Nco sequence. Optionally hypoxia responsive enhancer/promoters or other inducible promoters are also introduced into the vector. These transduced SS progenitor cells differentiate into mature SS erythrocytes that synthesize and secrete these tumoricidal molecules. After systemic injection, these SS cell populations localize in tumor vasculature where they adhere to endothelium, induce vaso-occlusion, secrete and efflux their tumoricidal molecules resulting in tumor cell cytolysis.

The present invention also contemplates replacing the AA hemoglobin gene with nucleic acids encoding the SS hemoglobin in autologous hematopoietic progenitor cells obtained from tumor bearing patients. In another embodiment, the SS hemoglobin replacing the AA hemoglobin is similarly linked to tumoricidal transgenes as described above. These transduced SS hematopoietic precursors are injected into their original donor where they induce tumor vaso-occlusion and a tumoricidal response. Such SS cells constitute a constantly recirculating and regenerating source of sickle cells cytotoxic for tumor cells.

### DETAILED DESCRIPTION

The present invention constitutes the use of mature sickle erythrocytes (SSRBCs) or sickle cell erythroid progenitor cells (SSEPCs) transduced with transgenes encoding tumoricidal agents, oncolytic viruses and regulatory RNAs (collectively TTORs) capable of inducing a cytolytic or cytostatic tumor cell response when administered in vivo to a tumor bearing host. To produce these cells the TTORs are integrated into the coding region of a novel β-globin lentiviral vector and transfected into nucleated SSEPCs at any stage of erythroid developmental. The TTORs are fully expressed in the nucleated SSEPC population as it differentiates and also in mature enucleated SSRBCs wherein the TTOR gene products are produced along with SS hemoglobin. In the present invention, the SSEPCs and SSRBCs with or without transduced with TTORs are obtained from induced pluripotent stem cells (SSiPPSCs) or embryonic stem cells (SSESCs) derived from patients with sickle cell anemia. The iPPSCs and ESCs are also obtained from donors with AA hemoglobin which is removed and replaced with SS hemoglobin using specific gene editing technology.

In one embodiment, these SSRBCs or SSEPCs derived from iPPSCs or ESCs cells devoid of TTORs are administered to tumor bearing animals and patients as an autologous or allogeneic transplant (Animal Tumor Models and Examples I and IV). This is based on the findings that recurrence of certain tumors in mice can be prevented by exposure to a continuous source of SSRBCs. In another embodiment, the SSRBCs or SSEPCs housing TTORs derived from iPPSCs or ESCs are administered to patients with established tumors as periodic transfusions (Animal Tumor Models and Examples 2, 3, 5). This is based on the findings that such transfused cells bearing TTORS induce a tumoricidal response against established tumors in mice. In another embodiment SSEPCs and SSRBCs are subjected to graded photosensitization ex vivo to induce hemolysis at a specific time after in vivo administration coincident with maximum deposition of SSRBCs or SSEPCs in the tumor.

### Sickled Erythrocytes and Nucleated Erythroid Precursors for Targeted Delivery of Tumoricidal Agents

The present invention presents a remedy for the problems of specificity and efficacy. It provides a natural cell, the erythrocyte of sickle cell anemia and variants which the inventor has observed to have a proclivity to deposit in the tortuous neovasculature of tumors. This property of the sickled cells provides exquisite specificity for tumor tissue. In the hypoxemic environment of tumors, SS hemoglobin depolymerizes resulting an increase in membrane rigidity. In this state the SS cells are insufficiently malleable to make their way through the channels of the tortuous tumor vasculature. Under these conditions, the cells also upregulate expression of ligands/receptors such as aᵥV₄, CD36 and VCAM-1 which bind to their cognate receptors/ligands on the tumor endothelium where they induce local hemostasis and clot formation.

This invention differs from other therapies in the field in that the therapeutic is a natural product of nature. The native SS erythroblast targets microvasculature of virtually all solid tumors without relying on the presence of tumor specific antigens, antibodies, markers, overexpressed receptors or ligands or specific signaling molecules. It does not induce the immunosuppression of chemotherapy or the acute toxicity of various toxins.

Perhaps the most significant problem in therapeutics of cancer is specificity and targeting of anti-tumor agents into tumor tissue while sparing normal tissues. Tumor specific monoclonal antibodies specific for tumor associated antigen/receptors alone or with an attached conjugate have had some success but to date have shown only a modest increase in survival as well as numerous untoward side effects. Hence there is a quest for additional agents that can specifically target tumors with more potent tumor killing effects and less morbidity. A recent autopsy of a patient with SA disease and terminal cervical carcinoma showed the selective deposition of sickled erythrocytes in the vasculature of the tumor while the peripheral blood smear had no sickled elements. It was proposed that the hypoxemic environment of the tumor predisposed to the polymerization of hemoglobin and sickling in the tumor not seen in the normally oxygenated peripheral blood (Milosevic et al., Gyn Oncol 83; 428-431 (2001).

The interior of carcinomas is significantly hypoxemic relative to normal tissues (see Table 1). Under these conditions of deoxygenation erythrocytes from patients with sickle cell anemia SS hemoglobin polymerizes leading to a sickled morphology, The cells become structurally rigid and are insufficiently flexible to negotiate the tortuous and angular microcirculation of the tumor. In addition, SS erythrocytes become adherent to the tumor microvasculature due to upregulated expression of integrin complex a₄b₁ and CD36 which bind to endothelial VCAM-1 and platelet thrombospondin respectively. The least dense sickle erythrocytes are especially involved in hypoxia-sensitive adherence while secondary trapping of SS4 (dense cells) occurs in post capillary venules. In this way the SS red cells aggregate and obstruct tumor microvessels.

Because of its marked tortuosity and hypoxemia relative to normal tissues, the neovasculature of carcinomas is especially is well suited for selective deposition and aggregation of SS erythrocytes. Under hypoxic conditions, inflammatory cytokines such as TNF, various interleukins and lipid-mediated agonists (prostacyclins) commonly produced by patients with carcinomas also increase the adhesive and hemostatic properties of tumor neovasculature and promote the adherence of SS cells (Table 1).

TABLE 1
Oxygenation of tumors and normal tissues

| | | Median normal |
|---|---|---|
| Tumor type | Median PO₂ (pt. no.) | PO₂ (pt. no.) |
| Glioblastoma | 4.9 (10) | Nd |
| | 5.6 (14) | Nd |
| Head and Neck Carcinoma | 12.0 (30) | 40 (14) |
| | 14.7 (23) | 43.8 (30) |
| | 14.6 (66) | 51.2 (65) |
| Lung Cancer | 7.5 (17) | 38.5 (17) |
| Breast Cancer | 10.0 (15) | Nd |
| Pancreatic Cancer | 2.7 (7) | 51.6 (7) |
| Cervical Cancer | 5.0 (8) | 51.0 (8) |
| | 5.0 (74) | Nd |
| | 3.0 (86) | Nd |
| Prostate Cancer | 2.4 (59) | 30.0 (59) |
| Soft Tissue Sarcoma | 6.4 (34) | Nd |
| | 18.0 (22) | |

### Nucleated Sickle Cells for Transfection of Tumoricidal Agents

Nucleated erythroid precursors from patients with sickle cell anemia are the useful in the claimed subject matter. Because they are endowed with nuclei, they are readily transduced with the therapeutic oncolytic viruses and nucleic acids encoding toxins, toxin-tumor specific antibodies, -diabodies, -nanobodies and other therapeutic molecules The hemoglobin of these cells polymerizes and they undergo characteristic morphological deformation in the form of fine, fragile, elongated spicules consisting of highly organized and tightly aligned hemoglobin fibers in the protruded regions. The nucleated erythroblasts have a larger volume than mature red cells and have more dilute hemoglobin which is confined mostly to the cytoplasm. Nevertheless, under partial or complete deoxygenation they behave much like mature SS red cells, i.e., they deposit and aggregate in the tumor microcirculation.

Nucleated erythroid precursors can be readily obtained in abundance by culture peripheral blood erythrocytes with erythropoietin (Fibach et al., Exp Hematology 26:319-319 (1998); Fibach et al., Blood 73: 100-103 (1989). Peripheral blood (10-20 mL) is drawn from patients with sickle cell anemia. Mononuclear cells isolated by centrifugation on a gradient of Ficoll-Hypaque are cultured according to a two phase liquid culture procedure. In phase 1, the cells are cultured for 7 days in α-minimal essential medium supplemented with 10% fetal calf serum (both from Gifco, Grand Island N.Y.), cyclosporine A (1 μg/ml) (Sandoz, Basel, Switzerland) and 10% conditioned medium collected from bladder carcinoma 5637 cultures. In phase 2, the nonadherent cells are recultured in α-medium supplemented with 30% fetal calf serum, 1% deionized bovine serum albumin, 1×10⁵M 2-mercaptoethanol, 1.5 mM glutamine, 1×10⁻⁶M dexamethasone, and 1 U/mL human recombinant erythropoietin (Ortho Pharmaceutical Co., Raritan N.J.). Cultures are incubated at 37° C. in an atmosphere of 5% CO, to air with extra humidity. Cell morphology is assessed microscopically on cytocentrifuge-prepared slides (Shandon, Cheshire, UK) stained with alkaline benzidine and Giemsa.

Nucleated Sickle Cells Transfected with Tumoricidal Transgenes

Nucleated erythroid precursors or progenitors from patients with sickle cell anemia are the useful in the claimed subject matter. Because they are endowed with nuclei, they are readily transduced with the therapeutic oncolytic viruses and nucleic acids encoding toxins, toxin-tumor specific antibodies, -diabodies, -nanobodies and other therapeutic molecules. The hemoglobin of these cells polymerizes and they undergo characteristic morphological deformation in the form of fine, fragile, elongated spicules consisting of highly organized and tightly aligned hemoglobin fibers in the protruded regions. The nucleated erythroblasts have a larger volume than mature red cells and more dilute hemoglobin which is confined mostly to the cytoplasm. Under partial or complete deoxygenation they behave much like mature SS red cells, i.e., their sickle hemoglobin polymerizes, they deposit and aggregate in the tumor microcirculation.

Nucleated erythroid precursors/progenitors can be readily obtained in abundance from peripheral blood erythrocytes (Fibach E et al., Exp Hematology 26:319-319 (1998); Fibach E et al., Blood 73: 100-103 (1989); Panzenbock B et al., Blood 1998 92:3658-3668; Arcasoy M O & Jiang X Brit. J. Haematol. 130:121-129 (2005)). Peripheral blood (10-20 mL) is drawn from patients with sickle cell anemia. Mononuclear cells isolated by centrifugation on a gradient of Ficoll-Hypaque are cultured according to a two phase liquid culture procedure. In phase 1, the cells are cultured for 7 days in α-minimal essential medium supplemented with 10% fetal calf serum (both from Gifco, Grand Island N.Y.), cyclosporine A (1 μg/mL) (Sandoz, Basel, Switzerland) and 10% conditioned medium collected from bladder carcinoma 5637 cultures. In phase 2, the nonadherent cells are recultured in α-medium supplemented with 30% fetal calf serum, 1% deionized bovine serum albumin, 1×10⁵M 2-mercaptoethanol, 1.5 mM glutamine, 1×10⁻⁶M dexamethasone, and 1 U/mL human recombinant erythropoietin (Ortho Pharmaceutical Co., Raritan N.J.). Cultures are incubated at 37° C. in an atmosphere of 5% CO₂, with extra humidity. Cell morphology is assessed microscopically on cytocentrifuge-prepared slides (Shandon, Cheshire, UK) stained with alkaline benzidine and Giemsa. Nucleated erythroid precursors/progenitors are obtained from bone marrow or erythroid cells or stem cells. They are also obtained from established erythroid and stem cell lines. The desired nucleated progenitor cells are generally CD34+. All of these cells are identified, isolated and enriched using methods well established in the art.

Cell banks are prepared consisting of ABO and Rh typed, nucleated sickle precursor cells, transfected with the appropriate tumoricidal agents under control of the HRE. Cell banks can also include mature SS, SA and other sickle variants cells incorporating anaerobic bacterial spores, Listeria, S. aureus or tumoricidal drugs for use in patients with solid tumors. Thus it is feasible to use nucleated erythroid precursor cells for transfection of HRE and nucleotides encoding tumoricidal agents.

### SS Cells, Nucleated SS Erythroblasts and Erythroleukemia Cells Transduced by Nucleic Acids Encoding Oncolytic Viruses, Tumoricidal Toxins, Toxin Homologues Toxin-Antibody Proteins, Superantigens, Superantigen Homologues Superantigen Conjugates.

The present invention contemplates the transduction of the SS cells, SS erythroblasts and erythroleukemia cells by oncolytic viruses, plasmids encoding oncolytic viruses, tumoricidal toxins, toxin-antibody proteins, therapeutic antibodies or antibody fragments and cytokines all optionally under the control of the hypoxia responsive element (HRE). The HRE has been reported in the 5′ or 3′ flanking regions of hypoxia responsive molecules VEGF and EPO and phosphoglycerate kinase promoter and several other genes and is indispensable for their hypoxia-induced transcriptional activation. The core consensus sequence is (A/G) CGT (G/C) C (Forsythe, J A et al., Mol. Cell. Biol. 16:4604-4613 (1996); Levy, A P, J. Biol. Chem. 270, 13333-13340 (1995); Gupta, M et al., Blood 96, 491-497 (2000)).

HIF-1, a key transcription factor that binds to HRE, regulates the expression of various hypoxia-responsive molecules such as EPO. HIF-1 is composed of a 120-kDa O₂-regulated β subunit and a 91- to 94-kDa constitutively expressed a subunit. HIF-1 activity depends mainly on the intracellular level of HIF-1α protein, which is regulated in inverse relation to the oxygen concentration by an oxygen-dependent enzyme, prolylhydroxylase 2 (PHD2). Under hypoxic conditions, the a subunit is stabilized because of the lack of proline hydroxylation and accumulates. Stabilized HIF-1α translocates into the nucleus and forms an HIF-1 complex with the almost ubiquitously expressed HIF-1β. The HIF-1 complex binds to hypoxia response elements (HREs) found in enhancers or promoters of hypoxia-inducible genes.

In the present invention, the HRE is used preferably in concatenated form of up to 15 or more repeats (Prentice H et al., Cardiovasc Res. 35:567-74 (1997)). It is activated at tissue oxygen partial pressures of 1% and with more recent improvements in concatenation to 2-2.5%. The latter pO₂ is well within the range of most carcinomas. The HRE can be used with various promoters (complete or minimal) of which the CMV appears to be the most potent under hypoxic conditions. The present invention contemplates that the HRE as a key promoter in the virus or vector used to transduce SS erythrocytes. The inventor contemplates that preferably the HRE is incorporated into SS erythroblasts ex vivo before administration of the erythrocytes to the patient. After the latter cells are administered to a living body with tumor or suspected tumor (microscopic metastases) they localize in tumor microvasculature. Under hypoxemic conditions of the tumor microenvironment, nucleic acids encoding oncolytic viruses and/or tumoricidal transgenes are activated.

The present invention contemplates sickled erythroid precursors optionally containing the HRE found for example in the EPO and VEGF genes to control the transcription of various tumor selective viruses and tumoricidal agents. When this sickled erythrocyte is trapped in hypoxemic tumor microvasculature, the HRE optionally activates the synthesis of the tumoricidal viruses and proteins producing a targeted tumor killing response. The present invention contemplates any inducible promoter operatively linked to nucleic acids encoding any tumoricidal transgenes or constitutive genes including but not limited to tumoricidal viruses, toxins, toxin-tumor specific antibody fusions, cytokines including but not limited to TNFα and IFNγ, lytic agents including but not limited to perforins, granzyme, hemolysins, holotoxins, autolytic toxins and key constitutive enzymes as useful and functional. Inducible promoters and transcriptional control elements useful in the present invention include but are not limited to estrogen and steroid responsive promoters, tetR gene, radiation inducible promoters such as EGR-1, thyroglobulin promoter, albumin promoter, heat responsive promoters, heavy metal responsive promoters, tissue-restricted transcriptional control elements include the α₁-antitrypsin and albumin promoters (hepatocyte-selective), tyrosine hydrolase promoter (melanocytes), villin promoter (intestinal epithelium), glial fibrillary acidic protein promoter (astrocytes), myelin basic protein (glial cells), and the immunoglobulin gene enhancer (B lymphocytes), tumor-selective promoter elements include α-fetoprotein (hepatoma), DF3/MUC 1 (breast and other carcinomas), thyroglobulin (thyroid carcinoma), prostate-specific antigen (prostate carcinoma), and carcinoembryonic antigen (breast, lung, and colorectal carcinomas), DF3/MUC 1 promoter, Myc/Max family. The erythroid precursor can accept, encode and deliver plasmids of any kind including those expressing tumoricidal viruses and manmade virus constructs with tumoricidal activity

The present invention contemplates adeno- or self-replicating RNA viral vectors incorporated into SS erythrocytes, their nucleated precursors, sickle hemoglobin variants, erythroleukemia cells and activated by their HREs under hypoxic conditions of the tumor microvasculature leading to hemolysis and shedding of the HRE-containing adeno- or Sindbis virus. By placing the viral gene essential for transcription optionally under the hypoxia responsive promoter element (HRE), viral proliferation is activated under conditions of severe hypoxia present in most tumors and carcinomas in particular. Notably, the HRE also confers these viruses with a natural tropism for tumor cells exhibiting high levels of HIF-1. The ability of this promoter to preferentially direct transcription in hypoxic cells can be assessed by producing a plasmid that contains the promoter operatively linked to several well known fluorescent coding sequences. The HRP-fluorescent marker construct is used to establish stable sublines from tumor cell lines: Cells grown in normoxic conditions do not express the marker whereas cells from stably transduced sublines exposed to hypoxic conditions (with oxygen tension at 0.5 to 1.5%) showed excellent expression of the marker.

Conditional replication competence using the HRE constructs results in selective vector replication in sickle cells localized in the hypoxic tumor microcirculation. An oncolytic virus (preferably tumor selective/specific) linked to the HRE proliferates and hemolyzes the erythrocyte. The virus with an HRE-viral construct has an affinity for tumor cells with high levels of HIF-1. Other excellent viral constructs such as d11530 and Sindbis viruses by themselves have an affinity for tumor cells deficient in p53 and laminin receptors respectively and are preferably linked to an HRE enhancer. Virus is shed from the burst erythrocyte to infect tumor cells with high levels of HIF-1 (and/or P53 deficiency or laminin receptors). High replication of the vector is achieved in the tumor cells while replication in surrounding non-neoplastic cells is minimal.

For genes that are upregulated in response to hypoxia, wherein the precise sequence that confers hypoxia inducibility is unknown, the responsive sequence can be identified by methods known to the average artisan. Within a candidate promoter region, the presence of regulatory proteins bound to a nucleic acid sequence is detected with variety of methods well known to those skilled in the art (Ausubel et al, ed. Short Protocols in Molecular Biology. New York: Green Publishing Associates and John Wiley & Sons. P. 26-33 (1992)). Briefly, in vivo footprinting assays demonstrate protection of DNA sequences from chemical and enzymatic modification within living or permeabilized cells. Likewise, in vitro footprinting assays show protection of DNA sequences from chemical or enzymatic modification using protein extracts. Nitrocellulose filter-binding assays and gel electrophoresis mobility shift assays (EMSAs) track the presence of radiolabeled regulatory DNA elements based on provision of candidate transcription factors. Computer analysis programs, for example TFSEARCH version 1.3 (Yutaka Akiyama: “TFSEARCH: Searching Transcription Factor Binding Sites”) can also be used to locate consensus sequences of known transcriptional regulatory elements within a genomic region.

A hypoxia inducible promoter is concatamerized, polymerized or combined with additional elements to amplify transcriptional activity and mRNA translation in response to hypoxia. The hypoxia inducible promoter comprises 5-10 tandem copies of the HRE from the human VEGF or EPO gene linked to the CMV minimal promoter or many other promoters well known in the art.

A hypoxia inducible promoter of the presently claimed subject matter is responsive to non-hypoxic stimuli that can be used in combined therapy. For example, the mortalin promoter is induced by low doses of ionizing radiation (Sadekova S et al., Int J Radiat Biol. 72:653-60 (1997)), the hsp27 promoter is activated by 17 beta-estradiol and estrogen receptor agonists (Porter J et al., J Mol Endocrinol. 26:31-42 (2001)), the HLA-G promoter is induced by arsenite, and hsp promoters can be activated by photodynamic therapy (Luna M C et al., Cancer Res. 60:1637-44 (2000)). Thus, a hypoxia inducible promoter can comprise additional inducible features or additional DNA elements. Virus administration can be provided before, during, or after radiotherapy; before, during, or after chemotherapy; and/or before, during, or after photodynamic therapy. Moreover, a hypoxia inducible promoter can be derived from any biological source such as the human VEGF or EPO promoter that can direct efficient hypoxia inducible expression as in bovine pulmonary artery endothelial (BPAE) cells (Liu Y et al., Circ Res. 77:638-43(1995)).

### Tumoricidal Transgenes

In order to more efficiently kill a tumor cell that is infected with an adenovirus vector a transgene is provided. A transgene comprises a therapeutic gene, including, but not limited to a tumor suppressor gene, an apoptosis-inducing gene, an anti-angiogenic gene, a suicide prodrug, converting enzyme gene, a bacterial toxin gene, an antisense gene, a tumor suppressor gene, an immunostimulatory gene, or combinations thereof. A “transgene” A transgene includes a gene that is partly or entirely heterologous (i.e., foreign) to the organism from which the cell was derived, or can be a nucleotide sequence identical or homologous to a gene already contained within the cell.

Transgenes may also be delivered by replication-competent vectors which may be noncytopathic. Transgenes comprise nucleic acids encoding a polypeptide having a therapeutic biological activity. Exemplary therapeutic polypeptides include but are not limited to TNFα, IFNγ, and immunostimulatory molecules, various cell toxins such as superantigens or superantigen homologues alone or fused or conjugated to a tumor targeting agent, tumor suppressor gene products/antigens, suicide gene products, and anti-angiogenic factors and antibodies, or prodrug-activating enzymes that release well-defined cytotoxins on reduction in hypoxic cells such as nitrobenzyl phosphoramidate mustards, nitroheterocyclic methylquaternary salts, cobalt(III) complexes and indoloquinones (see Mackensen et al., Cytokine Growth Factor Rev. 8:119-28 (1997); Walther et al., Mol Biotechnol. 13:21-8 (1999); Kirk et al., Human Gene Ther. 11:797-806 (2000)) and references cited therein. In addition the transgene can express a ligand such as hergulin which binds overexpressed human epidermal growth factor receptor (HER). The RNA alphaviruses exemplified by the Sindbis virus which selectively targets overexpressed laminin receptors on tumor cells may be incorporated into sickled erythrocytes or erythroblasts optionally under control of the HRE or promoters. Upon lysis of the sickled erythrocyte by the virus, free virus is shed into the tumor microenvironment where it can selectively target surrounding tumor cells. A suicide gene encoding a protein that causes cell death directly, for example by inducing apoptosis, is referred to as an “apoptosis-inducing gene” and includes but is not limited to TNFα (Idriss et al., Microsc Res Tech. 50:184-95 (2000)), TRAIL (Srivastava Neoplasia 3:535-46 (2001)), Bax, and Bcl-2 (Shen et al., Adv Cancer Res. 82:55-84 (2001)).

Other genes that encode proteins that kill cells directly include bacterial toxin genes, which are normally found in the genome of certain bacteria and encode polypeptides (i.e. bacterial toxins) that are toxic to eukaryotic cells. Bacterial toxins include but are not limited to diphtheria toxin, pseudomonas exotoxin A and superantigens (Frankel et al., Curr Opin Invest Drugs 2:1294-301(2001)). A list of superantigens. The wild type superantigens, their homologues and fusion proteins useful in this construct are disclosed in the instant application with a preference for staphylococcal enterotoxins of the enterotoxin gene complex (egc). The wild type superantigens, staphylococcal enterotoxins A, B, C, D, E, G H, I, J, K L M, N, O P, Q, R, S, U useful in this invention and their fusion proteins along with superantigens homologues and fusion proteins defined structurally by FASTA z value >13 and functionally as T cell mitogenicity the TCR vβ region are disclosed in U.S. Pat. No. 7,776,822 B (columns 5-35) incorporated in entirety by reference with their references. A particularly preferred superantigen fusion protein for use in this invention is the SEA homologue SEA/E-120 and related SEA homologues linked recombinantly to a tumor specific targeting agent such as the 5T4 single chain fv fragment disclosed in U.S. Pat. No. 7,125,554.

Additional suicide genes encode a polypeptide that converts a prodrug to a toxic compound. Such suicide prodrug converting enzymes include, but are not limited to the HSV-tk polypeptide, which converts ganciclovir to a toxic nucleotide analog (Freeman et. al., Semin Oncol. 23:31-45 (1996); cytosine deaminase, which converts the non-toxic nucleotide analog 5-fluorocytosine into a toxic analog, 5-fluorouracil (Yazawa et al. World J Surg 26:783-9 (2002); and cytochrome p450, which converts certain aliphatic amine N-oxides into toxic metabolites (Patterson L H Curr Pharm Des. 8:1335-47 (2002).

Additionally, a suicide gene can encode a polypeptide that interferes with a signal transduction cascade involved with cellular survival or proliferation. Such cascades include, but are not limited to, the cascades mediated by the Flt1 and Flk1 receptor tyrosine kinases (reviewed in Klohs et al., Curr Opin Oncol. 9:562-8 (1997)). Polypeptides that can interfere with Flt1 and/or Flk1 signal transduction include, but are not limited to, a soluble Flt1 receptor (s-Flt1; Shibuya M Int J Biochem Cell Biol. 33:409-20 (2001) and an extracellular domain of the Flk-1 receptor (ex-Flk1; Lin P et al., Cell Growth Differ. 9:49-58 (1998)).

When entering the hypoxic tumor microcirculation, the sickled erythrocyte adheres to the tumor vasculature and the HRE is activated inducing the formation of nucleotides encoding the hemolysins which hemolyze the erythrocyte releasing oncolytic virus into the tumor site. Sickle cell deposition in tumor vessels leads to reduced SS cell velocity, upregulation of endothelial VCAM-1, TNFα, and p-selectin, trapping of additional sickled cells and micro-occlusion of the tumor microvasculature.

For proteins such as Pseudomonas exotoxin A and superantigens, 4-9 copies of the EPO HRE consensus sequence (SEQ ID NO:1) (CCGGGTAGCTGGCGTACGTGCTGCAG) are optionally inserted into the pβgal-promoter plasmid between SmaI and HindIII sites (CLONTECH) upstream of the simian virus 40 (SV40) or CMV promoter. The expression cassette (nine copies of optional EPO HRE, SV40 minimal promoter, LacZ gene, and SV40 polyadenylation signal) is cloned into an AAV vector between two inverted terminal repeats to generate the AAVH9LacZ vector as shown.

AAVH9 Pseudomonas Exotoxin A or AAVH9 SEG is generated by replacing LacZ gene in AAVH9LacZ with Pseudomonas exotoxin A or Staphylococcal enterotoxin G respectively. The HRE is optionally fused to various nucleic acids encoding tumoricidal proteins including but not limited to superantigens (preferably staphylococcal enterotoxins G, I, M, N, O), superantigen antibody conjugates, pseudomonas exotoxins (exotoxin A being the best characterized), verotoxins and/or subunits, diptheria toxin, pertussis toxin, complement membrane attack complex, perforins, holins, S. aureus autolysins, granzymes, perforins, tumor specific antibodies, chemokines, cytokines and chemoattractants. Likewise a hemolysin such as S. aureus alpha toxin, Listeria or E. Coli hemolysin are fused to the HRE to facilitate the internal lysis of the SS erythroid precursors under hypoxic conditions.

Truncated and mutant forms of bacterial toxins such as superantigens and superantigen antibody conjugates are useful in this invention are shown in FIG. 3 of Kreitman R J & Pastan I Adv Drug Deliv Rev 31: 53-88 (1998) as described below Amino acid 607 of PE and the remaining carboxyl terminal amino acids 608-613 are depicted. Pseudomonas exotoxin (PE) contains domains Ia (amino acids 1-252), I (amino acids 253-364), Ib (amino acids 365-399) and III (amino acids 400-613) are shown below. In PE4E, basic amino acids at positions 57, 246, 247 and 249 of PE are replaced by glutamate residues. In PE40, domain 1a has been removed from PE. In PE38, amino acids 365-380 have been removed from domain Ib of PE40. In PE38KDEL, the carboxyl terminal amino acids REDLK of PE38 have been replaced with KDEL. PE35 contains methionine followed by amino acids 281-364 and 381-613 of PE, and the only cysteine residue in PE35 is shown at position 287. Diphtheria toxin (DT) contains a methionine preceding amino acids 1-5 (GADDV). DT contains an A chain (amino acids 1-193) and a B chain (amino acids 194-535). In DAB486, amino acids 486-535 of DT are removed, and in DT388 or DAB3g, amino acids 389-535 of DT are removed. All of these forms are useful in the claimed invention.

Any tumor specific antibody, fv, Fab fragment either single or double chain or tumor targeting ligand e.g., EGF, chemokine receptor ligand specific for any and all human tumors listed herein is useful in the present invention. The mesothelin tumor specific monoclonal antibody which has been fused to PE40 and shown broad anti-tumor activity is particularly preferred. Likewise, any other tumoricidal molecules or molecules that promote tumor killing, e.g., Panton-Valentine leukocidin (PVL) including but not limited to ricin, diphtheria toxin, pertussis toxin either alone or coupled to a tumor specific targeting structure is useful in this invention. A targeting device and tumor toxin are conjugated as fusion proteins or biochemically cross linked using well established technology.

Likewise, nucleic acids encoding monoclonal antibodies specific for epitopes expressed on tumor cells, tumor parenchyma or tumor vasculature can be transfected into the SS erythroid progenitor or SS pluripotent erythroid stem cells or erythroleukemia cells using recombinant vectors well established in the art. An example of one such monoclonal antibody is Avastin which specific for VEGF receptors on tumor endothelium. SS cells or erythroleukemia cells localized in the tumor vasculature release the VEGF-specific monoclonal antibodies into the tumor mileau. The tumor neovasculature is within easy reach of the recombinant antibodies and expresses epitopes expressed on tumor endothelium and endothelial matrix such as VEGF and laminin-α5. In this way, anti-angiogenic therapy such anti-VEGF is concentrated at the site of its cognate ligand in the tumor neovasculature, produces an increase in the therapeutic index of the drug and reduction in its systemic side effects.

A typical pharmaceutical toxin composition for intravenous administration includes about 0.1-10 mg per patient per day. Dosages from 0.1 ug to about 100 mg per patient per day may be used particularly if the agent is administered to a secluded site and into the circulatory or lymph system such as into a body cavity or into a lumen of an organ. This amount of toxin can be readily generated in approximately 10-100 cc of sickled erythrocytes once activated under hypoxemic conditions. Actual methods for preparing administrable compositions will be known or apparent to those skilled in the art are described in more detail in such publications as Remington's Pharmaceutical Science, 10th ed. Mack Publishing Company, Easton, Pa. (1995).

siRNA, shRNA, miRNA

RNA interference (RNAi) is a highly conserved gene silencing mechanism that uses double-stranded RNA (dsRNA) as a signal to trigger the degradation of homologous mRNA. The mediators of sequence-specific mRNA degradation are 21- to 23-nt small interfering RNAs (siRNAs) generated by ribonuclease III cleavage from longer dsRNAs. A short (usually 21-nt) double-strand of RNA (dsRNA) with 2-nt 3′ overhangs either end. Twenty-one-nucleotide siRNA duplexes trigger specific gene silencing in mammalian somatic cells without activation of the nonspecific interferon response.

Transfection of an exogenous siRNA is enhanced by introduction of a loop between the two strands, thus producing a single transcript, which can be processed into a functional siRNA. Such transcription cassettes typically use an RNA polymerase III promoter (e.g. U6 or H1), which usually direct the transcription of small nuclear RNAs (snRNAs) (U6 is involved in gene splicing; H1 is the RNase component of human RNase P). The resulting siRNA transcript is then processed by Dicer. In one embodiment of the present invention, SS cells or SS erythroblasts are transduced in vitro with vectors encoding siRNAs directed to RNA species that induce or contribute to tumoricidal activity. The therapy is applicable to carcinomas, sarcomas, gliomas, melanomas and lymphomas/leukemias. The present invention contemplates as useful any of these vectors containing an FMG optionally under control of the HRE or other suitable promoter. Typically, the SS cells and sickled erythroblasts are transfected in vitro with the siRNA using siRNA expression vectors or PCR products. Synthetic siRNAs chemically synthesized or in vitro transcribed siRNAs can be transfected into cells or injected into mice. A self-replicating cytopathic alphavirus vector is preferred such as the SINrep5 which expresses Sinbis virus structural proteins that recognize laminin receptors on tumor cells. The siRNA of choice is integrated into the cloning region of these viruses or cotransfected with them.

When administered parenterally, to tumor bearing hosts, the transfected SS cells or SS erythroblasts undergo viral induced lysis, shedding the virus containing the siRNA to infect surrounding tumor cell selectively. Alternatively, the FMG containing vectors can fuse with and transfer siRNA to the target tumor or endothelial cells.

Alphaviruses with self replicating replicons containing an FMG such as the Sindbis and SFV that can lyse the SS erythroblast carrier or fuse with and infect adjacent tumor cells are preferred but other tumor specific viruses shown in Table 1 of U.S. Pat. No. 8,524,218 B2 and Table 2 of U.S. Pat. No. 8,418,117 B2 such as d11150 and those targeting HIF1-a in tumor cells are also useful. Optionally, the VP22 or any other other cell penetrating peptide that promotes cell to cell transfer is cointegrated into the silencing. RNAs. Such RNA conjugates include siRNA, shRNAs or miRNAs which can be incorporated into viruses or integrated into the β-globin lentiviral vector for transfection of SSEPCs or SSiPPSCs as described below. The alphavirus vector (pRep5) together with the siRNA is a useful example. DNA fragments encoding VP22 are isolated by digesting pcDNA3-VP22, respectively, with XbaI and PmeI restriction enzymes. These isolated DNA fragments are further cloned into the corresponding XbaI and PmeI sites of the SINrep5 vector to generate SINrep5-siRNA-VP22 constructs.

An adenovirus is one of the most well-known viral vectors for gene delivery. Intratumoral injection of an adenovirus encoding the hypoxia-inducible factor-1 (HIF-1α)-targeted siRNA had a significant effect on tumor growth when combined with ionizing radiation (Zhang et al., Cancer Res. 64:8139-42 (2004)). The very same construct optionally containing the FMG is used to infect SS cells, SS erythroblasts or erythroleukemia cells that are lysed by the virus leading to viral shedding into surrounding tumor tissue. Other viruses are useful including but not limited to the alphaviruses and any other virus that can maintain viability in and the ability to transfect tumor cells from within the carrier SS cells. The virus selectively infects hypoxic tumor cells expressing HIF-1 and induces apoptosis via siRNA targeting of HIF-1.

In addition to siRNAs, shRNAs and micoRNAs produce excellent gene silencing as described below. All of these RNAs can be packaged into oncotropic/oncolytic viruses which can be used to infect SS erythroblasts. siRNAs, shRNAs or micoRNAs are engineered to target any gene overexpressed in tumor cells and lyse the tumor cell by inactivating key genetic function(s) or by endogenous self-replication.

Candidate target genes for knockdown mediated by siRNAs, shRNAs or miRNAs are selected from several key oncogenes, antiapoptotic genes such those encoding VEGF, heme oxygenase, HIF1-α, arginine/nitric oxide, Bcl-2, p53 or tumor promoting genes, including growth and angiogenic factors or their receptors. As a matter of course any gene that promotes the growth of a cancer cells such as antiapoptotic gene(s), p53 and defective suppressor oncogenes whether or not it is overexpressed, mutated or translocated in tumor cells, is a useful targets for silencing. In this context, initial in vitro studies have demonstrated effective silencing of a wide variety of mutated oncogenes such as K-Ras, mutated p.53, Her2/neu and bcr-abl. Silencing sRNA software is available for design of effective RNA sequences (Takeshita F., Cancer Sci 97: 689-696 (2006)). In this context, genes that confer resistance to treatment such as the ABC transporters and MDR gene(s) are also contemplated as targets for siRNAs, shRNAs or miRNAs.

Gene knockdown by transfection of exogenous siRNA is made permanent and impervious to cell multiplication by creating an expression vector for the siRNA. The siRNA sequence is modified to introduce a short loop between the two strands. The resulting transcript is a short hairpin RNA (shRNA), which can be processed into a functional siRNA by Dicer in its usual fashion. Typical transcription cassettes use an RNA polymerase III promoter (e.g., U6 or H1) to direct the transcription of small nuclear RNAs (snRNAs) (U6 is involved in gene splicing; H1 is the RNase component of human RNase P). The resulting siRNA transcript is then processed by Dicer.

The activity of siRNAs and shRNAs is largely dependent on its binding ability to the RNA-induced silencing complex (RISC). Binding of the duplex siRNA or shRNA to RISC is followed by unwinding and cleavage of the sense strand with endonucleases. The remaining anti-sense strand-RISC complex can then bind to target mRNAs for initiating transcriptional silencing.

A microRNA. (miRNA) is a small non-coding RNA molecule (ca. 22 nucleotides) that are abundant in many human cell types, conserved in eukaryotic organisms and also found in plants, animals, and some viruses. The human genome may encode over 1000 miRNAs targeting about 60% of protein coding mammalian genes. Seventy percent are intragenic and are located in different regions of the genome. They are invariably expressed together with the host gene because both are controlled by the same promoter region. Forty percent of all miRNAs are organized in clusters, and the miRNAs in each cluster usually regulate a common pathway. miRNA has multiple roles in negative regulation (transcript degradation and sequestering, translational suppression) and also involvement in positive regulation (transcriptional and translational activation). Their main function is to establish and maintain the differentiated state of many cell types by regulating key biological processes such as cell proliferation and apoptosis. In contrast to RNA interference technology, which is capable of silencing only one gene or a few genes belonging to the same gene family an important characteristic of miRNA expression modulation is that a single miRNA is capable of silencing several genes. Estimates of the average number of unique messenger RNAs that are targets for repression by a typical microRNA range from 7 to several hundred. This turns the therapeutic modulation of aberrantly expressed miRNAs into a powerful tool for the treatment of cancer and carcinogenesis.

miRNAs are encoded by eukaryotic nuclear DNA in animals and by viral DNA in certain viruses whose genome is based on DNA. As much as 40% of miRNA genes may lie in the introns of protein and non-protein coding genes or even in exons of long nonprotein-coding transcripts and are regulated together with their host genes. Animal miRNAs are able to recognize their target mRNAs by as few as 6-8 nucleotides (the seed region) at the 5′ end of an miRNA. A given miRNA may have multiple different mRNA targets, and a given target might similarly be targeted by multiple miRNAs. miRNAs function via base-pairing with complementary sequences within mRNA molecules. Such mRNA strands are silenced because they are unable to be translated into proteins by ribosomes.

miRNAs (miRNA) are noncoding, regulatory small RNAs of ˜22 nucleotides (nt) in length Canonically, miRNAs are transcribed in an RNA polymerase II-dependent fashion to produce a primary-miRNA transcript (pri-miRNA), consisting of one or more miRNA-containing stem-loop structures, the majority of which are embedded in introns. The pri-miRNA secondary structure is recognized by the nuclear microprocessor, which consists of the ribonuclease (RNase) III enzyme Drosha and the essential double-stranded RNA (dsRNA) binding protein DiGeorge syndrome critical region gene 8 (DGCR8). The microprocessor generates a ˜60-70-nt precursor miRNA (pre-miRNA) containing a 2-nt-39 overhang, a characteristic of RNase III-mediated processing. Subsequently, Exportin-5 (ExpS) translocates the pre-miRNA into the cytoplasm in a Ran-GTP-dependent fashion. Once in the cytoplasm, a second RNase III enzyme, Dicer, recognizes the distinct terminus of the pre-miRNA and cleaves ˜22 nt from the end, generating a duplex RNA flanked by 2-nt-39 overhang. The efficiency of Dicer processing is enhanced by the dsRNA binding proteins Taractivating RNA binding protein 2 (TRBP2) and/or a TRBP2 homolog, PKR activating protein (PACT). One strand of the duplex, denoted the mature strand, is selected to associate with an Argonaute (AGO1-4) protein, a core component of the RNA induced silencing complex (RISC), while the other strand (star strand) is degraded. Once loaded into RISC, the miRNA sequence serves as a guide to identify target mRNA through partial complementarity between the mRNA and the miRNA seed sequence (nucleotide 2-8 from the 59 end of the mature miRNA), resulting in translational repression and/or mRNA deadenylation in a process collectively termed post-transcriptional silencing (PTS).

In vertebrates, the lack of endogenous cytoplasmic RNA-dependent RNA polymerases (RdRps) in vertebrates prevents the production of bona fide siRNAs. To circumvent the absence of RdRp, Sindbis virus, that replicates exclusivelyin the cytoplasm, is grafted with a locus encoding pri-miRNA dsRNA. The viral encoded pri-miRNA dsRNA retains the ability to be processed by Dicer and is functional in RNA silencing. To accomplish this, the pri-mRNA locus is inserted into an extra, nonessential subgenomic fragment of Sindbis virus. The Sindbis virus encoding pri-miRNA dsRNA is dependent on cytoplasmic Dicer and Drosha RNA splicing, producing up to 55,000 copies per cell entirely independent of nuclear microprocessing (Shapiro et. al., RNA 16:2068-2074 (2010); Shapiro et. al., RNA 18:1338-1346 (2012)); Varble et al., RNA Biology 8: 190-194 (2010)).

The pri-miRNA loaded viruses may be transfected into SSEPCs, SSiPPSCs and SSRBCs using well established methods of lipofection, electroporation or brief incubation with the target cells in vitro at MOIs of 5-25 (Chen et al., PLoS One 3(6): e2360. (2008); Shapiro et. al., RNA 16:2068-2074 (2010)). The Sindbis virus encoding pri-miRNA is preferred for transfection of enucleated SSRBCs because it does not rely on the nuclear microprocessor ribonuclease (RNase) III enzyme Drosha and the essential double-stranded RNA (dsRNA) binding protein DiGeorge syndrome critical region gene 8 (DGCR8) to produce the miRNA. In vivo, Sindbis virus encoding pri-miRNA is transferred from the SSRBC to target tumor cells by bleb formation and fusion whereas transport from the SSEPCs or SSiPPSCs to the target cells is effected by exosomal transfer via the virologic synapse. Once in the target cell, the virus continues its production of miRNA that silences or replaces the key RNAs involved in tumor growth and metastases. Any RNA and DNA virus encoding pri-miRNA is useful in this invention including but not limited to vesicular stomatitis virus, reovirus, semilike Forest virus, tick borne encephalitis virus, West Nile Virus, Particularly useful are wild type or engineered viruses that exhibit tumor tropism and/or oncolytic activity.

Non-coding siRNAs, shRNAs and miRNAs are also effective as oligonucleotides for tranduction of SSEPCs, SSiPPSCs or SSRBCs by lipofection or electroporation methods well described in the art (Chen et al., PLoS ONE 3(6): e2360. (2008)). DNAs encoding the pri-miRNA can also be integrated into the lentiviral β-globin vector for transfection of SSEPCs or SSiPPCs as described herein. In this method, the pri-mRNA is transcribed in the transduced cells and their progeny including mature SSRBCs even after enucleation.

Many deregulated miRNAs are associated with the development of various cancers, establishment of cancer aggressiveness, invasiveness and metastatic capacity and resistance to anti-cancer treatments. The miRNA expression profiles have been analyzed in different cancer types including leukemia, lymphoma, glioblastoma, neuroblastoma, papillary thyroid carcinoma, esophageal, lung, breast, liver, pancreas, gastric, colorectal, ovarian, prostate, kidney, and bladder cancers. A recent example is the critical role of downregulated miRNAs in cancer is the miR-29 family which plays an important role in regulating cell proliferation, differentiation, apoptosis, migration, and invasion. Loss of miR-29 function has been observed in leukemia, melanoma, liver, colon, cervical, and lung cancer. Conversely, tumor suppressor miRNAs such as let-7 miR-17-92 cluster, let-7 and miR-155 that inhibit the expression of tumor suppressor oncogenes are usually overexpressed or amplified in tumor cells and contribute to tumor development. Thus, both loss of function or gain of function of miRNAs contributes to cancer initiation and progression.

Importantly, such miRNAs, shRNAs and siRNAs can be used to target cancer stem cells and prevent their renewal during and after treatment. Such cancer stem cells (CSCs) within a tumor that possess the capacity to self renew and produce heterogeneous lineages of cancer cells that comprise the tumor. Numerous signal pathways may participate in regulating CSC functions, including Wnt/β-catenin, Notch, and Sonic hedgehog homolog (SHH). The canonical Wnt cascade has emerged as a critical regulator of stem cells and activation of Wnt signalling has also been associated with various cancers. More recently, cutaneous CSC maintenance has been shown to be dependent on β-catenin signaling. Let-7 has been shown to be markedly reduced in breast tumor-initiating cells (BT-ICs) and increased with cell differentiation. Infection of BT-ICs with lentivirus-mediated let-7 reduced cell proliferation, mammosphere formation, and the proportion of undifferentiated cells in vitro, and tumor formation and metastasis in NOD/SCID mice, while antagonizing let-7 by antisense oligonucleotides enhanced in vitro self-renewal of non-BT-ICs. Further studies showed that let-7 regulated multiple BT-IC stem cell-like properties by silencing more than one target, H-Ras or HMGA2]. Subsequently, miR-205 and miR-22 were found to be highly expressed in mammary progenitor cells, while let-7 and miR-93 were depleted. Let-7 sensors enriched self-renewing populations, and enforced let-7 expression induced loss of self-renewing cells from mixed cultures. These results suggest that miRNAs play important roles in CSC proliferation, differentiation and tumor formation. Examples of miRNAs found in various CSCs and the phenotypes of the CSCs for various cancers are given in Table 2 and 3 respectively of Xia HP J Cancer Mol 4: 79-89 (2008) incorporated by reference with their references in entirety.

Correction of these miRNAs and their regulated gene network can changes the behavior of cancer cells. Consequently, two therapeutic strategies used in miRNA modulation are (a) the replacement of miRNAs downregulated or missing in the cancer cell and (b) the introduction of synthetic antagomiRs to inhibit overexpressed miRNAs. Providing the missing let-7 miRNA in the lung cancer cells significantly restored normal function in lung cancer cell lines and lung cancer tissues. Synthetic antisense oligonucleotides (antagomiRs) complementary to endogenous mature miRNAs overexpressed miRNA expression in tumor cells have also been useful in reducing unwanted miRNAs. For example, the miRNA let-7 represses expression of oncogenes Ras, Myc and HMGA-2, and let-7 levels were found to be low and in primary tumor cells derived from 100 patients diagnosed with ovarian cancer. let-7 expression was also reduced in mammosphere-derived cancer stem cells when compared with normal breast or non-selected tumor cells, indicating that let-7 may prevent proliferation of cancer-initiating stem cells.

Combining miRNA regulation with gene therapy allows targeted and expression of transgenes to specific cells or tissues. Such strategies incorporate miRNA target sites in the 3′ UTR of the therapeutic transgene, preventing its expression in cells that express the corresponding miRNA. The transgene will be expressed in the intended cell type, in which the miRNA expression is suppressed. Likewise, tissue or organ specificity of the transgene can be enhanced by inclusion miRNA(s) in the transgene(s) that is(are) organ specific preventing expression of the transgene selectively in cells of that tissue or organ. Similarly, a lentiviral transgene targeting a specific cell type can be restricted to the desired cells by inserting an miRNA in the 3′UTR that prevents transgene expression in the undesired cells. In the case of pluripotent stem cell populations, differentiated cells can be obtained for transplantation back into the patient by using a differentiation-induced miRNA which turns off differentiation of pluripotent cells allowing proliferation of only differentiated cells of a specific lineage (Broderick et al., Gene Ther. 18: 1104-10 (2011)).

Delivery of therapeutic naked siRNA, shRHA or miRNA to tumor cells in vivo has been hampered by their limited stability in serum, rapid blood clearance, off-target effects, and poor cellular uptake. Chemical modifications (locked nucleic acid, 2′-O-methylation, etc.), and nanoparticle delivery systems have been developed to overcome these challenges have shown little progress. Delivery by nanoparticles and liposomes have to date shown no convincing therapeutic benefit in vivo.

In the present invention miRNAs are incorporated into the lentiviral β-globin vector for transduction of SSEPCs, SSiPPCs and SSRBCs. Examples of such therapeutic miRNAs are provided in Table 1 of Xia H P J Cancer Mol 4: 79-89 (2008) and Table 1 of Schoof et al., Am J Cancer Res 2:414-433 (2012). The Tables in the latter two references also provide xamples of genes targeted by the miRNAs and their respective tumor cells. Both references and their references are incorporated by reference in entirety.

One or more siRNAs, shRNAs or miRNAs with different specificities may be used in the same carrier SS cell. Alternatively, one or more species of silencing nucleotides may be carried in several transduced SS cells and administered simultaneously in a single infusion or each may be administered sequentially in multiple infusions. Infusion or cycles may be repeated every 3-12 days for up to 6 months. The preparation of silencing RNAs specific for and capable of inactivating the target genes is described in U.S. patent application Ser. No. 13/367,797.

Tumor Suitable for Treatment by Inventions Described in this Application

The compositions of the claimed invention are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

The compositions are also be useful for the prevention of metastases from the tumors described above either when used alone or in combination with radiotherapeutic, photodynamic, and/or chemotherapeutic treatments conventionally administered to patients for treating disorders, including angiogenic disorders. Treatment of a tumor with surgery, photodynamic therapy, radiation and/or chemotherapy is followed by administration of the compositions to extend the dormancy of micrometastases and to stabilize and inhibit the growth of any residual primary tumor or metastases. The compositions can be administered before, during, or after radiotherapy; before, during, or after chemotherapy; and/or before, during, or after photodynamic therapy.

Types of Sickle or Sickle Thalassemia Erythrocytes/Erythroblasts and Thalassemia Erythroblasts Useful in the Inventions in this Application

The present invention contemplates that erythrocytes or erythroblasts or erythroid progenitor cells or pluripotent erythroid stem cells from patients with any form of sickle hemoglobinopathy are useful. These include erythrocytes or erythroblasts from hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Indeed, any erythrocyte or erythroblasts with or without sickle hemoglobin expressing receptors capable of binding to tumor neovasculature are useful in the inventions described herein. Particularly useful are those cells which express hemoglobin S in combination with other types of hemoglobin. Both mature and nucleated forms of these cells are useful. In addition, the present invention contemplates that normal or leukemic erythrocytes or their nucleated progenitors transduced with hemoglobin genes from patients with hemoglobinopathies to produce a cell that behaves substantially like an SS or SA erythrocyte or erythrocyte precursor is useful. The present invention also contemplates that normal or sickle erythrocytes or sickle variants, e.g., HbSC cells, and nucleated progenitors which are upregulated by hormones, cytokines, biologically active agents, drugs, chemical or physical treatments to express adhesive properties or to enhance expression of adhesive properties are also useful in this invention.

### SS Erythrocytes/Erythroblasts Producing Tumoricidal Agents: Vectors Comprising Tumoricidal Transgene(s) Under Control of the β-Globin Promoters/Enhancers, Locus Control Region, DNase Hypersensitivity Sites.

The present invention contemplates SS erythroid progenitors or erythroleukemia cells transfected with a lentiviral vector in which the viral LTR is deleted and the β-globin promoter/enhancer and a tumoricidal transgene are inserted preferably in frame rendering these cells capable of synthesizing a tumoricidal molecule. Erythroid progenitor cells transfected with this vector express the tumoricidal transgene in quantity. By including an erythroid-specific transcriptional signal in the vector along with a tumoricidal transgene, the present invention exploits the genetic programming of the red blood cell which devotes almost its entire synthetic capabilities to the production of hemoglobin. SS progenitors transduced with this vector are also capable of differentiating into mature SS RBCs which continue to synthesize the tumoricidal molecule even after their nuclei have been extruded.

Virtually no other cell in the body is devoted to the synthesis of a single protein product to the extent that erythroid cells are committed to the synthesis of hemoglobin. The developing red blood cell down-regulates the expression of the vast majority of its genes in order to focus its synthetic machinery on the production of hemoglobin; in doing so it loses its nucleus and most other organelles and becomes, essentially, a membrane-bound packet of hemoglobin. The domination of the red cell's synthetic capabilities is effected, to a large extent, by a formidable surge of transcription of globin genes upon commitment to differentiation. By redirecting the red cell transcriptional signals toward inducing expression of a tumoricidal transgene by substituting it for the β-globin coding region, the present invention exploits the genetic programming of erythroid cells and thereby provides an efficient method for producing a tumoricidal transgene in quantity.

In humans, the β-globin gene cluster is located on chromosome 11 and comprises one embryonic (e) two fetal (^{σ}γ and ^{A}γ) and two adult δ- and β-globin genes, which reside within approximately 50 kb of chromosomal DNA in the order 5′-£-σγ-^{A}γ-δ-β-3′ (Fritsch et al. 1980, Cell 19:959-972). Expression of the human 3-like globin genes is confined to only erythroid tissue during defined stages of development. They produce β-globin proteins at very high levels. Full expression of the β-globin gene requires inclusion of the promoter and several enhancer sequences and sequences at the extreme ends of the human β-globin locus including the erythroid-specific DNase I super-hypersensitive sites fused upstream of the human β-globin gene.

Four regulatory elements are required for appropriate expression of the human β-globin gene include: (I) a β-globin specific promoter element; (ii) a putative negative regulatory element, and (iii and iv) two downstream regulatory sequences with enhancer-like activity, one of which is located in the second intron of the β-globin gene and the other located approximately 800 basepairs (bp) downstream of the gene (Behringer et al. 1987. Proc. Natl. Acad. Sci. 84:7056-7060(1986); Hesse et al., Proc. Natl. Acad. Sci. U.S.A. 83:4312-4316 (1986) (iv) sites that are super-hypersensitive to DNase I digestion 6-22 kilobases (kb) upstream of the ε-globin gene and 19 kb downstream of the β-globin gene (Tuan et al. Proc. Natl. Acad. Sci. 82:6384-6388 (1985); Forrester et al. Proc. Natl. Acad. Sci. 83:1359-1363 (1986)) (v) major DNase I hypersensitive sites, HS I, HS II, and HS IV situated upstream of the β-globin gene define locus activation regions dramatically enhance 3-like globin gene expression specifically in erythroid cells.

The preferred vector of the present invention is of lentiviral origin modified as follows. The β-globin promoter/enhancer (2.3-kb) along with a tumoricidal transgene of choice are subcloned into the pWPT-GFP vector replacing the EF1a promoter and green fluorescent protein (GFP). This self-inactivating (SIN) vector contains a deletion in the U3 region of the 3′ long terminal repeat (LTR) from nucleotide 418 to nucleotide 18 that inhibits all transcription from the LTR. The vector also contains a tumoricidal transgene which replaces the coding region of the β-globin gene. It also comprises the β-globin promoter (266 bp), the PstI 3′ globin enhancer (260 bp), and a 375-bp RsaI fragment deletion of IVS2. The lentiviral-globin LTR contains in addition to the 3′globin enhancer, the β-globin promoter and the β^{AS3} globin gene, a 3′ SIN deletion, a ψ packaging signal, splice donor and acceptor sites, RRE indicating Rev-responsive element, cPPT/CTS indicating central polypurine tract or DNA flap/central termination sequence and WPRE specifying woodchuck hepatitis virus post-transcriptional regulatory element. DNase 1 hypersensitive sited (HS) fragments 5′ HS4, 3, and 2 are amplified by polymerase chain reaction (PCR) from a 22-kb fragment of the LCR. Nucleotide coordinates from GenBank accession no. U01317 are: HS4 592-1545, HS3 3939-5151, and HS2 8013-9215. The entire HS4 3.2 β-globin gene construct is verified by sequencing. The full length lentiviral vector containing a tumoricidal transgene vector is modified from Levasseur T et al., Blood 102: 4312-4319 (2003) in the present invention by substituting the tumoricidal transgene for the β^{AS3}-globin gene resulting in production of a tumoricidal transgene.

In a particularly preferred embodiment a modified lentiviral vector is employed as described in Levasseur T et al., supra (2003). A tumoricidal transgene is substituted for the coding region of the β-globin gene in this vector under control of the β-globin promoter/enhancer that will drive expression of tumoricidal transgenes. Any tumoricidal transgene can be inserted at this site with the expectation of robust expression. The portion of the β-globin locus which includes the enhancer found in the second intron of the β-globin gene as well as part of the third exon of β-globin and the enhancer located 3′ to the human β-globin gene, poly A sequence and a β-globin Nco sequence are retained for coding stabilization. Also retained in the vector are the locus control region (LCR) and DNAase hypersensitivity regions.

The staphylococcal enterotoxin (SEB) is attractive candidate for insertion into the β-globin coding region. The coding region of SEB is described in Jones C J et al., J Bacteriol 166: 16629-33 (1986); Ranelli D M et al., Proc. Natl. Acad. Sci. 82: 5850-5854 (1985). The whole plasmid described in Ranelli et al., supra 1985 contains a 6-kb HindIII fragment from that includes the SEB gene cloned into the HindIII site of pUC 19. The cloned fragment is described in Ranelli et al supra 1985 pages 5850-5854 (attached). The pSK401 plasmid is similar to that described in Ranelli et al., supra 1985 except that the vector is high copy pUC19 in place of pBR322. The sequence of the SEB gene and surrounding region is described in Jones and Khan supra, pages 29-33 paper (attached). The ATG codon of SEB precursor is at position 244 of the sequence and the mature SEB coding sequence starts at position 325.

| SEB Gene Ranelli DM et al., Proc. Natl. Acad. Sci. | |
|---|---|
| U.S.A. 82, 5850-5854 (1985) | |
| (SEQ ID NO: 2) | |
| 1 | GAACTAGGTA GAAAAATAAT TATGAGAAAA CACTATGTTG TTAAAGATGT |
| 51 | TTTCGTATAT AAGTTTAGGT GATGTATAGT TACTTAATTT TAAAAGCATA |
| 101 | ACTTAATTAA TATAAATAAC ATGAGATTAT TAAATATAAT TAAGTTTCTT |
| 151 | TTAATGTTTT TTTAATTGAA TATTTAAGAT TATAACATAT ATTTAAAGTG |
| 201 | TATCTAGATA CTTTTTGGGA ATGTTGGATA AAGGAGATAA AAAATGTATA |
| 251 | AGAGATTATT TATTTCACAT GTAATTTTGA TATTCGCACT GATATTAGTT |
| 301 | ATTTCTACAC CCAACGTTTT AGCAGAGAGT CAACCAGATC CTAAACCAGA |
| 351 | TGAGTTGCAC AAATCGAGTA AATTCACTGG TTTGATGGAA AATATGAAAG |
| 401 | TTTTGTATGA TGATAATCAT GTATCAGCAA TAAACGTTAA ATCTATAGAT |
| 451 | CAATTTCTAT ACTTTGACTT AATATATTCT ATTAAGGACA CTAAGTTAGG |
| 501 | GAATTATGAT AATGTTCGAG TCGAATTTAA AAACAAAGAT TTAGCTGATA |
| 551 | AATACAAAGA TAAATACGTA GATGTGTTTG GAGCTAATTA TTATTATCAA |
| 601 | TGTTATTTTT CTAAAAAAAC GAATGATATT AATTCGCATC AAACTGACAA |
| 651 | ACGAAAAACT TGTATGTATG GTGGTGTAAC TGAGCATAAT GGAAACCAAT |
| 701 | TAGATAAATA TAGAAGTATT ACTGTTCGGG TATTTGAAGA TGGTAAAAAT |
| 751 | TTATTATCTT TTGACGTACA AACTAATAAG AAAAAGGTGA CTGCTCAAGA |
| 801 | ATTAGATTAC CTAACTCGTC ACTATTTGGT GAAAAATAAA AAACTCTATG |
| 851 | AATTTAACAA CTCGCCTTAT GAAACGGGAT ATATTAAATT TATAGAAAAT |
| 901 | GAGAATAGCT TTTGGTATGA CATGATGCCT GCACCAGGAG ATAAATTTGA |
| 951 | CCAATCTAAA TATTTAATGA TGTACAATGA CAATAAAATG GTTGATTCTA |
| 1001 | AAGATGTGAA GATTGAAGTT TATCTTACGA CAAAGAAAAA GTGAAATTAT |
| 1051 | ATTTTAGAAA AGTAAATATG AAGAGTTAGT AATTAAGGCA GGCACTTATA |
| 1101 | GAGTACCTGC CTTTTCTAAT ATTATTTAGT TATAGTTATT TTTGTTATAT |
| 1151 | CTCTCTGATT TAGCATTAAC CCCTTGTTGC CATTATAGTT TTCACCAACT |
| 1201 | TTAGCTGAAA TTGGGGGATC ATTTTTATCT TTACTATGGA TAGTTACTGT |
| 1251 | GTCGCCGTTT TTAACGATTT GTTTCTCTTT TAATTTGTCA GTTAATTTTT |
| 1301 | TCCATGCATC ATTTGCGTCA AACCTATTTC CATTTGGATT TATTCTTGAC |
| 1351 | AAATCAATTC TTTTAACACT ATCGGTATTA ATCGGCTTGT TATTAAAATT |
| 1401 | ACTAAGTTCA TCTAAATCAG CTGTACCCGT AATACTACTT TCGCCACCAT |
| 1451 | TATTTAAATT GTACGTAACA CCAACTGTCT CATTTGCTGT TTTATCGATA |
| 1501 | ATATTTGCTT CTTTCAAAGC ATCTCTTACA TTTTTCCATA AGTCTCTATC |
| 1551 | TGTTATTTCA GAAGCCTTTG CAACGTTATT AATACCATTA TAATTTGAAG |
| 1601 | AAGAATGAAA ACCTGAACCT ACTGTTGTTA AAACTAAAGC ACTTGCTATC |
| 1651 | AATGTTCTTG TTAATAGTTT TTTATTCATT TTATTTTCTC CTATAACTTA |
| 1701 | TTTGCAATCG AT |

The above described sequence contains a 27 amino acid signal sequence near the amino-terminal, a stop codon, an Nco 1 linker and BamH linker inserted into the β-globin Nco1 site. The insertion site consists of an Nco1 ATG. Alternatively a BamH modified site is prepared downstream (FIG. 4). Additional vectors comprising nucleic acids encoding superantigen homologue-tummor specific antibody conjugates as described by Forsberg et al U.S. Pat. No. 7,125,554 B2 incorporated in entirety by reference are also useful for integration into the lentiviral vector as described above.

The cloned enterotoxins B (entB) gene is subcloned into several known vectors such as the pC194 and pE194 as vector plasmids. It has also been subcloned in to the pHβ-actin-neo vector and transfected successfully into 4T1 carcinoma cells. These mammalian tumor cells expressed and secreted SEB and induced a tumoricidal response in vivo (Terman PCT/US1999/008399; Pulaski et al., Cancer Res. 60-2710-15 (2000)). Recombinant plasmids containing the entB gene in both orientations direct the synthesis of SEB, suggesting that the gene is being transcribed from its own promoter. The complete nucleotide sequence of the entB gene, including the 5′ and 3′ flanking regions is shown in FIG. 2 of Jones et al., supra 1986. Starting from an ATG codon at nucleotide 244, there an open reading frame (ORF) of 798 nucleotides terminates in a TGA stop codon at nucleotide 1042. This corresponds to the SEB precursor which consisted of 266 amino acids, with a calculated molecular weight of 31,400. The SEB precursor contains a putative signal sequence of 27 amino acid residues. The calculated molecular weight of the 239-amino acid mature SEB is 28,366. ATG codon at nucleotide 244 is the translational start site and is the first ATG in the first initiating codon upstream of the GAG codon (glutamic acid) at nucleotide 325 that encodes the NH2-terminal residue of SEB. The ATG codon is preceded by a strong Shine-Dalgarno sequence, AAAGGAG and a strong ribosome-binding sequence. The ATG codon at nucleotide 244 is signal peptide of 27 amino acids. The 5′ flanking region of the entB gene contains a possible −10 TATAT sequence which is an acceptable fit to the canonical TATAAT sequence. Forty nucleotides downstream from the TGA stop codon of the entB gene is a palindromic sequence that is a transcription terminator.

In a further experiment, the β-globin exons 1 and 2 and the intron 1 of the vector were replaced by a foreign transgene which efficiently transduced Sca-1⁺ progenitors, which were transplanted into lethally irradiated mice. To determine if a similar system could support a tumoricidal transgene, we replaced the β-coding region of the vector with the coding region of a staphylococcal enterotoxin (FIG. 13). Sca-1+ bone marrow cells from SS mice were transduced with this vector and used to reconstitute lethally irradiated hemoglobin AA mice. FIGS. 10,11 and 12 demonstrate efficient RFP, superantigen and SS expression in mature RBCs at 3 months post transplant. FACS analysis of the peripheral blood with antibody to the superantigen showed that a mean of 36% of the mature SSRBCs expressed the superantigen protein along with SS hemoglobin. Mice showed few ill effects of the treatment and several survived more than 8 months. To further test the tumoricidal effects of the superantigen-loaded SSRBCs, we inserted the coding sequences of two 3^{rd}-generation staphylococcal enterotoxin superantigens (SEG and SEI) into the lenti/HS2-4-globin vector. SEG and SEI coding sequences were connected with a short picornavirus 2A peptide sequence which we used successfully in our OSK reprogramming vector (13). This lenti/HS2-4 β-globin vector with the inserted SEG/SEI transgenes was used to transduce SS Sca-1⁺ bone marrow cells from sickle cell mice.

Other native SEs, SE homologues, SE fusion proteins and fragments with tumoricidal activity are also useful for insertion into the lentiviral vector in this invention. To qualify as an SE homologue or fragment of a wild type SE the molecule must demonstrate T cell mitogenicity in a conventional T cell proliferation assay and show a z>13 in FASTA when compared to the wild type SEs. Details of this methodology are given herein in the section on protein homologues.

Additional SE fragments and structural homologues are useful in this invention that have a z value >13 in FASTA and demonstrate direct tumor cell cytotoxicity versus carcinoma cell lines in vitro with or without the aid of MHCII antigen presenting cells. These fragments do not demonstrate T cell mitogenicity in a conventional T cell mitogenicity assay. In contrast to the wild type enterotoxins they are non-toxic in vivo. The present invention contemplates that the active site on SEC is homologous to the 130-160 amino acid fragments of wild type SEB. Any other molecule that is cytotoxic for tumor cells in an MHCII-independent manner and shows a z>13 in FASTA compared to the wild type SEB 130-160 fragment is also useful in this invention.

SEB 130-160 fragment Papageorgiou,
A. C. et al. J. Mol. Biol. 277: 61-79 (1998)

(SEQ ID NO: 3)

| | |
|---|---|
| 130 | rsitvrvfedgknllsfdvqtnkkkvtaqel |

SEC 157-187 (Bohach, G. A. et al.,
Mol. Gen. Genet. 209: 15-20 (1987))

(SEQ ID NO: 4)

| | |
|---|---|
| 156 | nvlirvyenkrntisfevqtdkksvtaqel |

The present invention envisions the incorporation of nucleic acids encoding Panton Valentine leukocidin (PVL) into the lentiviral vector under control of SS β-globin enhancer as described above. PVL induces cytolysis of polymorphonuclear leukocytes a key cell population in promoting endothelial and tumor cell death in the course of SS cell-induced tumor vaso-occlusion. Shortly after SS vaso-occlusion, PMNs are activated, recruited to and infiltrate sites of SS cell adherence to tumor endothelium resulting in endothelial cell injury and tumor cell death. The tumor endothelium not only promotes the recruitment of inflammatory leukocytes to sites of SS adherence but also sends additional activating signals that are crucial for vascular injury. Endothelial E-selectin-mediated signals are transduced via ESL-1 (E-selectin ligand) and locally activate the integrin α_{M}β₂ at the leading edge of crawling neutrophils. Activated α_{M}β₂ clusters mediate heterotypic interactions with circulating RBCs and platelets, which promote vascular occlusion and damage. The release of PVL via the β-globin gene in SS cells entrapped in tumor vasculature leads to up-regulation in PMNs of CD 1 1b/CD 18 glycoprotein, activation of phospholipase A2, release of oxygen metabolites, IL-18 and leukotriene B4 resulting in DNA fragmentation, cell lysis with release of proteases, oxidants and phospholipase. These agents induce necrosis of the proximate tumor endothelium and adjacent tumor cells

PVL has high cytolytic specificity for human polymorphonuclear cells and macrophages. PVL, leukocidins and staphylococcal γ-hemolysins are bicomponent toxins of Staphylococcus aureus. PVL and γ-hemolysin are composed of five separate and complete proteins termed “F” and “S”. Class S and F proteins are secreted separately as lytically inactive components but act synergistically on the target cell membrane to form membrane pores. The assembly of the both components on the surface of the PMN is required for it cytolytic activity.

Pore formation of the staphylococcal leukocidal toxins is associated with the assembly of a heptameric β barrel structure from the monomer pairs of S and F proteins, (e.g., LukS-PV/LukF-PV, H1gA/H1gB, H1gC/H1gB) which is the active pore-forming configuration of the toxin. The GM1 receptor on target PMNs and monocytes is recognized by soluble class S molecules (e.g., LukS) binds with high affinity. Binding of S components to cell membranes is requisite before binding of F components can take place.

S components consist of LukS-PV, H1gA (32 kDa), H1gC (32 kDa) with 63 to 75% identity, and class F components include LukF-PV, H1gB (34 kDa) with 70% identity. The PVL class F component (LukF-PV) are shared in common with γ-hemolysin. The target cell specificities of both bi-component toxins are mainly determined by the class S (Hlg2 for γ-hemolysin and LukS-PV for PVL) proteins. There are seven possible functional combinations of S and F components. All seven are leukocytolytic, however, the couples H1gC/LukF-PV and LukS-PV/H1gB show only leukotoxic properties. Two of the couples, LukS-PV/LukF-PV and H1gA-LukF-PV, also display dermonecrotic activity on rabbit skin. The two γ-hemolysin combinations, H1gA/H1gC and H1gA/H1gB, and the hybrid couple, H1gA+LukF-PV, induce both leukocytolysis and hemolysis.

In the rabbit VX 2 carcinoma model, VX2 fragments are implanted in the lateral thigh female rabbits 3-4 kg in body weight are used and as described in U.S. Pat. No. 6,340,461 which is herein incorporated in entirely by reference. Human mature SS erythrocytes or SS progenitors (2-10 ml) synthesizing the PVL gene via the lenteviral vector described above are injected intravenously three times weekly for two weeks. The treatment is started when the tumors have grown to at least 1 cm³. Control tumor bearing rabbits are treated with SS cells that do not contain the PVL gene. Tumor measurements in treated and control rabbits are evaluated statistically by methods well established in the art. Median survival of treated and control groups is also determined at an arbitrary time points such as 30, 60, 90 and 120 days after starting treatment and the groups compared statistically by established methodology as described in U.S. Pat. No. 6,340,461 incorporated in entirety by reference.

The present invention contemplates that wild type shiga toxins, Shiga toxin mutants (two of which are shown below) and chain B are useful for integration in the above lentiviral vectors and transfection into SS progenitor cells. The mature SS cell containing the shiga toxin genes deposits in tumor vasculature wherein it produces the toxin locally. These agents recognize globotriaosylceramide (Gb3) binding sites expressed on breast, ovarian and colon carcinoma and are capable of inducing tumor cell apoptosis. The B subunit is responsible for the binding of the holotoxin to GB3 on the target human tumor cells. After binding to the Gb-3 receptors, STxB enters cells through clathrin-independent or -dependent endocytosis and uses retrograde transport to deliver the A subunit to the cytosol. The A subunit causes endohydrolysis of the N-glycosidic bond at one specific adenosine on the 28S rRNA resulting in tumor cell apoptosis.

Shiga-like toxin contains a single subunit A and five copies of subunit B. There are three Gb3-binding sites in each subunit B monomer, allowing for a tighter binding to the target cell. Sites 1 and 2 have higher binding affinities than site 3. The A subunit is responsible for inhibiting protein synthesis through the catalytic inactivation of 60S ribosomal subunits. After endocytosis, the A subunit is cleaved by furin in two fragments, A1 and A2: A1 is the catalytically active fragment, and A2 is essential for holotoxin assembly with the B subunits. Mutant forms of the wild type toxin and the B chain alone are less toxic than the wild type Shiga toxin and are also useful in the present invention. Biologically active mutants or variants the wild type Shiga toxin qualify as authentic shiga toxin homologues if they demonstrate binding to the Gb3 ligand using well established assays in the art and show a z>13 in FASTA versus the wild type shiga toxin.

The present invention contemplates that additional tumoricidal transgenes are inserted into β-globin coding region of the lentiviral vector including but not limited to IFN-β, TNFα, IL-12 or the FAS death domain RIP and any biocompatible tumor killing molecule, tumor specific or tumor vascular endothelium (anti-VEGF), specific monoclonal antibody, superantigen, superantigen homologue, superantigen-tumor associated antibody, antibody fragment or receptor conjugates (Forsberg et al U.S. Pat. No. 7,125,554 B2; Shaw et al., Br J Cancer 96: 567-574 (2007); Antonsson P et al Semin Immunopathol 17:397-410 (1996)), anti-angiogenesis agents, therapeutic or tumor specific monoclonal antibodies and immunotoxins, anti-tumor growth factor inhibitors, suicide agents such as thymidine kinase, cytosine deaminase, drug or prodrug or any biocompatible small peptide, protein, protein or peptide homologue or conjugate that is directly or indirectly involved in therapeutic tumor killing.

One skilled in the art recognizes that many other vectors are capable of introducing a tumoricidal transgene or siRNAs into SS progenitor cells, erythroblasts or erythroleukemia cells under the β-globin promoter/enhancer and are useful in this invention. These include baculoviruses (Granqiero L et al., J Immunol Meth 25: 131-139 (1997)) and adenoviruses (Je T C Proc Natl Acad Sci 95:12509-12514 (1998)) and Sindbis virus (Koller D et al., Nat Med 19: 851-855 (2001; Boorsma M et al., Nat Biotech 18:429-432 (2000)). Several additional vector systems with tropism for erthyroid stem cells, progenitor cells or erythroblasts or erythroleukemia cells are useful given in Verhoeyen et al., J Gene Med 6:S83-S94 (2004) are also useful with the β-globin promoter/enhancer. DNA reaction products may be cloned using any method known in the art. A large number of vector-host systems known in the art may be used. Possible additional vectors include, but are not limited to cosmids plasmids or modified viruses, but the vector system must be compatible with the host cell used. Such vectors include, but are not limited to, bacteriophages such as lambda derivatives, or plasmids such as pBR322, pUC, or Bluescript (Stratagene) plasmid derivatives. Recombinant molecules can be introduced into host cells via transformation, transfection, infection, electroporation, etc. Additional lentiviral vectors in the art that accommodate both the β-globin promoter/enhancer and a tumoricidal transgene are given in Tiscornia G et al., Nature Protocols 1: 241-244 (2006); Pellinen R et al., Int J Oncol 25:753-62 (2004); Loimas S Gene Ther Mol Biol 5: 147-155 (2000) and siRNAs (Robinson D A Nat Genet 33: 401-486 (2003); Schomber T et al., Blood 103: 4511-4513 (2004)) and are useful in the present invention.

The above lentiviral vectors are produced by transient transfection into 293T cells. A DNA cocktail containing 5 μg envelope-coding plasmid pMD.G, 15 μg of the packaging plasmid pCM-VDR8.91 (which expresses Gag, Pol, Tat, and Rev) and 20 μg SIN transfer vector plasmid. A total of 2.5×10⁶ 293 T cells are seeded in 10-cm-diameter dishes containing Dulbecco modified Eagle medium (DMEM) with 10% fetal bovine serum (FBS) 24 hours prior to transfection of 293 cells. Forty micrograms of plasmid DNA are used for transfection of one 10-cm dish. Transfection medium is removed after 14 to 16 hours and replaced with DMEM/F12 without phenol red (Invitrogen, Carlsbad, Calif.) containing 2% FBS. Viral containing supernatant is collected after an additional 24 hours, cleared by low-speed centrifugation, and filtered through a 0.22-μm polyethersulfone filter (Millipore, Beford, Mass.). The virus is concentrated 1000-fold by one round of centrifugation at 26,000 rpm for 90 minutes at 8° C., resuspended into serum-free stem cell growth medium (SCGM) (Cellgenix, Freiburg, Germany) and allowed to incubate on ice for 2 hours before storage at −80° C. Virus titer is determined by infecting murine erythroleukemia (MEL) cells, plating individual cells into wells of a 96 well plate and assaying DNA from the cultures by PCR for the human globin gene.

The constructs are removed from vector sequences by digestion with the appropriate enzymes and isolated in low-gelling temperature agarose (FMC) gels. Gel slices are melted, extracted twice with buffered phenol, once with phenol/chloroform, and once with chloroform and precipitated with ethanol. After suspension in TE (10 mM Tris-HCl (pH 8.0), 1.0 mM EDTA), the fragments are again extracted with phenol, phenol/chloroform, and chloroform and precipitated with ethanol. The purified fragments are washed with 70% ethanol resuspended in sterile TE, and transfected into erythroid cells.

SS human (CD34+) or murine (TER119⁺ and CD71⁺ or SCA⁺, cKit⁺ and Lin⁻) erythroblasts/progenitors are used preferentially for transduction by the lentiviral vector containing the tumoricidal transgene described above although erythroid precursors at any stage of development or differentiation and erythroleukemia cells are useful in this invention. Transduction with this vector is carried out by suspending the SS erythroblasts/progenitors in IMDM medium containing 10 μg/mL dextran sulfate and 1% FBS. One thousand cells are infected at a MO1 of 30 in a total volume of 100 μL at 37° C. in 5% CO₂ for 4 hours. These cells are induced to differentiate into large scale numbers of mature SS RBCs by established methods in the art (Lu et al., Blood published online Aug. 19, 2008; doi:10.1182/2008-05-157198). Cells are initially suspended in Stemline II medium, mixed with blast-colony growth media (BGM)(5×10⁵ cells/ml), plated in 100 mm ultra low dishes (10 ml/dish) and expanded for 9-10 days in BGM. The addition of 20 ng/ml of βFGF and 2 ug/ml of the recombinant tPTD-HoxB4 fusion protein to BGM significantly enhances hematopoietic cell proliferation. HoxB4 protein promotes hematopoietic development in both mouse and human ESC differentiation systems. The grape-like blast colonies are usually visible by microscopy after 4-6 days, and expanded rapidly outward. In step 2, additional BGM is added to keep the density of blast cells at 1-2×10⁶ cells/ml. Erythroid cell differentiation and expansion is carried out from day 11-20. At the end of step 2, the cell density is often very high (≧2×10⁶/ml). Equal volumes of BGM, containing 3 units/ml of erythropoietin (EPO) (total EPO is 6 units/ml) without HoxB4, are added to supplement the existing BGM. The blast cells are further expanded and differentiated into erythroid cells for an additional 5 days. For further expansion, the erythroid cells are transferred into 150 mm Petri dishes and Stemline II-based medium containing SCF (100 ng/ml), Epo (3 units/ml) and 0.5% methylcellulose added every 2-3 days. When the cells reach confluence, the cells are split at a ratio of 1:3 to allow maximum expansion for an additional 7 days (cell density 2-4×10⁶/ml). Enrichment of SS erythroid cells is carried out on day 21. SS erythroid cells are diluted in 5 volumes of IMDM plus 0.5% BSA medium and collected by centrifugation at 1000 rpm for 5 minutes. The cell pellets are washed twice with IMDM medium containing 0.5% BSA, and plated in tissue culture flasks overnight to allow nonerythroid cells (usually the larger cells) to attach. The non-adherent cells are then collected by brief centrifugation.

The present invention contemplates that siRNAs, shRNAs or microRNAs are also incorporated into the lentivirus vector or other functional vectors by methods known in the art (Schomer et al., Blood 103:4511-4513 (2004)); Rubinson et al., Nat Gen 33: 401-406 (2003)). These siRNAs and microRNAs silence nucleic acids regulating enucleation, apoptosis, angiogenesis, metastases and those promoting synthesis of antiviral proteins such as IRF-3, NFκB, cJUN/ATF-2.

In another embodiment of the invention, two or more species of such recombinant tumoricidal DNA molecules or an siRNA or microRNA comprising different tumoricidal or tumoristatic genes may be co-introduced into cells in culture in order to produce a protein comprising multiple, distinct subunit proteins, each of which corresponds to one of the species of recombinant DNA molecules introduced. In particular, according to the invention, a protein of interest having more than one species of subunit may be produced in erythroid cells by a method comprising (i) introducing into SS erythroid cells as DNA transfected into an erythroid cell line more than one recombinant nucleic acid construct, each of which comprises a gene encoding a subunit of the tumoricidal molecule and at least one SS erythroid-specific DNase I hypersensitive site; (ii) growing the cells under conditions in which erythroid-specific gene expression occurs (in cell lines this may involve induction of differentiation). In a specific embodiment of the invention, recombinant DNA constructs comprising tumoricidal molecules together with at least one DNase I HS site are co-introduced into an erythroid cell in order to produce the tumoricidal molecule in quantity in the erythroid cells. It is preferable, in the case of multidomain molecules, to coinject both constructs into the same erythroid cell.

Specific signal sequences on the therapeutic transgenes that target intracellular polypeptides into the secretory pathway and facilitates exodus from the cell are useful in the present invention. Signal peptides have a common structure: a short, positively charged amino terminal region (n-region); a central hydrophobic region (h-region); and a more polar carboxy-terminal region (c-region) containing the site that is cleaved by the signal peptidase. A secretory peptide is selected from those given in the Signal Peptide Database http://proline.bic.nus.edu.sg/spdb, a repository of experimentally determined and computationally predicted signal peptides (Choo K H et al., BMC Bioinformatics, 6:249-257 (2005)). In the lentiviral expression vector, nucleic acids encoding the selected secretory signal polypeptide are positioned 3′ or 5′ preferably in frame to the β-globin promoter/enhancer and tumoricidal transgene. Nucleic acids encoding the secreted polypeptide may also be operatively-linked to one or more transcriptional regulatory elements. The host cells transformed or transfected with the lentiviral expression vector is therefore able to express and secrete the polypeptide tumoricidal molecules into the external environment. The same secretory signals are useful in the self-replicating vectors synthesizing tumoricidal transgenes, the oncolytic viral constructs and in vectors containing the SS β-globin promoter/enhancer used to transduce tumor cells and T cell described below.

To confer additional level of specificity to the β-globin promoter/tumoricidal transgene construct, the present invention contemplates that the hypoxia responsive enhancer (HRE) is positioned in frame with the β-globin promoter/enhancer and optionally concatenated as described above. This ensures that transcriptional activation of the β-globin promoter and its downstream transgene occurs selectively in the hypoxic environs of the tumor vasculature. Methodology for introducing the HRE to the lentiviral or other vectors is given above in the instant specification. The HRE is also useful in the self-replicating vectors synthesizing tumoricidal transgenes, the oncolytic viral constructs and the vectors comprising the β-globin promoter/enhancer used to transduce tumor cells and T cell described below.

Viral vector-mediated transduction of defined factors has been used to generate induced pluripotent stem (iPS) cells from embryonic or adult somatic cells in both mouse and human. iPS cells have been shown to be are equivalent to embryonic stem (ES) cells maintaining a full capacity for differentiation with the ability to form teratomas, generate chimeras, and contribute to the germline. This technology can be readily applied to many cell types in addition to fibroblasts as numerous cell types have been shown to be amenable to direct reprogramming including pancreatic beta cells, neural precursors, and terminally differentiated B cells. iPPSCs cell technology has emerged as the most promising method for cell-based therapies of regenerative medicine.

In the present invention, a humanized knock-in mouse model of sickle cell anemia is used in which the mouse α-globin genes are replaced with human α-globin genes, and the mouse β-globin genes are replaced with human α-globin and βS (sickle) globin genes. Homozygous mice for the human βS allele remain viable for up to 18 months but develop typical disease symptoms such as severe anemia due to erythrocyte sickling, splenic infarcts, urine concentration defects, and overall poor health.

Methods for Deletion of AA Genes and Insertion of the SS Genes into Normal Hematopoietic Progenitor Cells

Several efficient methods can be employed for deletion of AA hemoglobin and insertion of SS hemoglobin in hematopoietic progenitor cells from autologous or allogeneic sources. These include zinc finger and transcription activator-like effector (TALE) nuclease method, RNA interference (RNAi), exon skipping technology, and gene transfer. The following articles provide details on the methods disclosed in the section below and are incorporated by reference in entirety with their references: Hockemeyer D, Nature Biotech 29: 731-734 (2011); Lombardo A et al., Nature Biotech published online 28 Oct. 2007: doi 10.1038/nbt 353; Urnov et al., Nature Rev Genetics 11: 636-646 (2010); Carlson et al., Molecular Therapy-Nucleic Acids vol. 1 e3 (2012); Pawitan J A, Stem Cell International Article ID 498197 (2012); Carrol D, Genetics 188: 773-782 (2011).

### Zinc Finger Nuclease Method.

The zinc finger nuclease method is one of the efficient genetic editing methods. These proteins can be used to introduce targeted modifications into endogenous loci. These modifications include gene disruption (the targeted induction of minor insertions and deletions), ‘gene correction’ (the introduction of discrete base substitutions specified by a homologous donor DNA construct) and targeted gene addition (the transfer of entire transgenes into a native genomic locus). A Zn finger nuclease consists of a Zn finger domain and FokI endonuclease. The Zn finger domain contains Zn finger motifs that recognize and bind to a specific DNA sequence. The Fold endonuclease works as a dimer to cause a double-strand break (DSB) in the DNA. Therefore, Zn finger nucleases should work in pairs. One of the Zn finger motifs recognizes and binds to the sequence up stream and the other to the sequence down stream to the site to be cleaved by the endonuclease. In principle, a certain Zn finger nuclease can be engineered to recognize any specific sequence and to cause a DSB at any specific site. The DSB is then repaired by homologous recombination, which is facilitated by the presence of exogenous donor DNA homologous to the sequence to be repaired, or by error-prone non-homologous end joining. To deliver the Zn finger nucleases into a cell, an expression vector containing the Zn finger nucleases can be engineered. The results of this genetic editing may be either mutation repair or insertion of a certain DNA sequence, when a certain exogenous donor DNA is used, or error prone repair when no donor DNA is used, or deletion when two pairs of Zn finger nucleases are used and causing 2 DSB. Therefore, this method may be used to correct a mutation, or to insert or delete a certain DNA sequence.

The method has been used to develop the zinc finger component of active ZFNs for a number of endogenous targets in higher eukaryotic cells. Each zinc finger (ZF) is about 30 amino acids, which form a ββα-fold stabilized by hydrophobic interactions and the chelation of a zinc ion, and generally binds to three base pairs. Typically, arrays of 3-6 ZF modules are joined together to create a DNA-binding domain with specificity to 9-18 base pairs per ZFN monomer. ZFPs contain fingers for each component DNA triplet linking them into a multifinger peptide targeted to the corresponding composite sequence. Such fingers have been developed for most triplet sequences. ZFNs obviate the need for drug selection, extend the application of gene knockout to potentially any cell type and species for which transient DNA or mRNA delivery is available, and result in knockouts in 1-50% of all cells.

Double-strand DNA cleavage requires dimerization of two Fold nuclease domains. As this interaction is weak, cleavage by FokI as part of a ZFN requires two adjacent and independent binding events, which must occur in both the correct orientation and with appropriate spacing to permit dimer formation. The requirement for two DNA binding events enables specific targeting of long and potentially unique recognition sites (from 18-36 bp). Thus, ZFNs are used in pairs with specificity to opposing DNA strands that assemble on both sides of the targeted cleavage site.

The resulting double-strand breaks in a DNA sequence can be repaired by either of two mechanisms, non-homologous end joining (NHEJ) or HR. NHEJ often results in small deletions or insertions (indels) to cause missense and/or nonsense mutations that truncate or mutate the encoded protein. Consequently, NHEJ-mediated mutagenesis is used for targeted disruptions of genetic loci (e.g., gene knockout). Alternatively, HR allows for either precise modification of a target sequence or precise introduction of a specific sequence (e.g., a wild-type sequence that leads to gene repair) into the targeted site. In mammals, a double-strand DNA break can stimulate HR of an exogenous DNA sequence within about 100 base pairs of the double-stranded DNA break. Consequently, both targeting DNA cleavage close to a deleterious mutation and supplying either a double-stranded or single-stranded template DNA sequence can repair a damaged gene.

Strategies for Developing Zinc Finger Proteins with New Sequence Specificities

Several alternatives to modular assembly have been developed for identifying zinc finger proteins (ZFPs) with sufficient affinity and specificity for use in genome engineering. One approach, called the ‘OPEN’ system uses bacterial selections to identify finger combinations that will work well together. The method involves two distinct steps. First, multiple, parallel low-stringency selections are performed for binding of randomized fingers to each triplet in the targeted sequence. Mild conditions ensure that the resultant pools retain considerable diversity. Next, fingers from these pools are combinatorially linked and the products are selected at high stringency for binding to the final target. A second approach for identifying ZFPs with new specificities uses a bacterial selection system that is similar to OPEN but a different strategy for library construction 36. For each target triplet, a library is assembled that randomizes only a subset of residues at the zinc finger-DNA interface. At the remaining positions, specificity is achieved by the use of residues chosen for their ability to make especially well-understood base contacts. An alternative path for accommodating finger-finger cooperativity uses explicit checking for a particularly energetic and well-understood extra-triplet contact during evaluation of prospective designs. Other studies have developed finger design weightings or have sought improved success rates through the use of only a subset of available finger designs, which are chosen for their consistency of function. Another strategy is to use two-finger modules (instead of individual fingers) as the principle unit of DNA recognition. This approach enables optimization of finger junctions within each module for more cooperative and specific base recognition. Moreover, it reduces the number of untested finger-finger junctions in any new ZFP design and therefore the risk of a poor interaction between newly joined fingers. A four-finger ZFP contains just one new junction instead of three if assembled from one-finger units. This approach is used to make zinc finger nucleases (ZFNs) consisting of four, five or six zinc fingers for a range of applications (note that a five-finger ZFN is constructed using one one-finger and two two-finger units). Each of these methods has been successfully used to generate endogenously active ZFNs.

### CRISPR-Cas System

Recently, a natural RNA-guided DNA nuclease system has been discovered in bacteria and archaea. Analysis of clustered regularly interspaced short palindromic repeats (CRISPR) has elucidated a unique system for adaptive immunity by CRISPR-associated (Cas) proteins. The specificity of the relies on tightly bound CRISPR RNA (crRNA), which efficiently guides a nuclease to its target—a complementary DNA fragment. A type II CRISPR-associated nuclease (Cas9) causes specific double-strand breaks in a DNA target. Processing of its crRNA guide involves a second, trans-acting crRNA (tracrRNA) and a ribonuclease (RNase III). When loaded with both crRNA and tracrRNA (or an

artificial chimera of the two RNAs), the Cas9 nuclease cleaves a DNA fragment that is complementary to the exposed part of the crRNA. Dedicated cleavage of plasmids in vitro demonstrates the promise of CRISPR-mediated gene targeting as a more generic molecular engineering tool.

Cong et al. and Mali et al. (Cong L et al., Science 339: 819-823 (2013); Mali P et al., Science 339: 823-826 (2013) report different applications of the bacterial Cas9 nuclease for RNA-guided engineering of mammalian genomes. Both groups describe the functional expression of Cas9 in the nucleus of cultured human cells. Cas9 that is loaded with guiding crRNA and assisting tracrRNA can carry out programmed DNA cleavage, which is eventually partly repaired by nonhomologous end joining, an event that frequently results in small insertions and deletions. Whereas the wild-type Cas9 generates double-strand breaks, Cong et al. and Mali et al. used a Cas9 mutant in which one of the nuclease active sites is disrupted. This variant generates breaks in only one of the DNA strands, which should diminish nonhomologous recombination. Indeed, both groups demonstrate a reduction in off-target mutations due to insertions and deletions. In this context, distinct donor DNA fragments were successfully integrated at the site of cleavage. Moreover, the simultaneous introduction of two adjacent double-strand breaks resulted in efficient deletion of the intervening fragment. Notably, it has been shown that this also allows for scaling up by multiplex editing of distinct target loci. Similarly, doublestrand DNA breaks based on Cas9-mediated genome editing also works in human cells. Cas9 cleaving efficiencies depended mainly on the design of the chimeric RNA, indicating a means for further optimizing the editing performance of Cas9. High-fidelity target recognition is critical, since off-site nuclease activity can jeopardize the safety of the engineering operation; thus, long stretches of nucleotides should be specifically recognized. I n addition, adjusting the system's specificity toward new target sequences should be easy and affordable. This is a major advantage of the Cas9 system, as it merely requires changing the sequence of the guide RNA. Furthermore, the recombination is fast, efficient, and scalable. Compared to the most promising currently available genome editing systems (zinc finger domains and TALENs), the RNA-guided Cas9 nuclease probably is closest to meeting these requirements (Van der Oost J, Science 339; 768-770 (2013).

### Homology-Based Genome Editing

The second, mechanistically more complex pathway that can be invoked following a ZFN-induced DSB is called HDr. Whether spontaneous or induced by the I-SceI homing endonuclease or a ZFN3, a DSB is recombinogenic in cells of higher eukaryotes. Homology-based genome editing requires the simultaneous provision of a suitably designed, homology-containing donor DNA molecule along with the locus-specific ZFNs.

### Gene Correction (Allele Editing).

This approach allows the transfer of single-nucleotide changes and short heterologous stretches from an episomal donor to the chromosome following a ZFN-induced DSB; The endogenous repair machinery uses the extrachromosomal, investigator-provided donor as a template for repairing the DSB via the synthesis-dependent strand annealing process. This technique enables the study of gene function and/or the modeling of disease-causing mutations through the creation of a point mutation that is characteristic, for example, of a known disease predisposing allele or that disables a motif that is thought to be crucial for function. Such point mutations can be efficiently created at a specific position in the target gene.

### Gene Addition in Mammalian Cells.

The same approach allows the transfer of gene-sized heterologous DNA sequences from an episomal or linear extrachromosomal donor to the genome following a ZFN-induced DSB. This has been demonstrated using ZFNs directed against IL2RG in combination with donors carrying homology arms of 750 bp that flank transgenes positioned precisely between the ZFN recognition sites. In this study, ˜5% of chromatids acquired transgenes of up to 8 kb in length in the absence of selection for the desired event. ZFN-driven gene addition can now be applied at other loci and mammalian cell types.

### Gene Addition in Human ES and iPS Cells

The initial demonstration of ZFN-driven targeted gene addition to an endogenous locus in human ES cells used an integration-defective lentiviral vector to deliver both the ZFN expression cassette and the donor construct. Gene addition was observed at rates as high as 6% in the absence of selection and resulted in stable gene expression for at least 2 months, both in cultures that retain ‘sternness’ and following neuronal differentiation. The application of this approach in human mesenchymal stem cells yielded 50% targeted gene addition without selection. Furthermore, ZFNs delivered as plasmid DNA have been used in human ES and iPS cells to efficiently target a drug resistance marker to a specific gene and to generate novel allelic forms of three endogenous loci. In all of these studies, efficient, specific and stable gene addition was achieved and the cells retained characteristics of pluripotency.

### TALE Nuclease Method.

Recent work on transcription activator-like effectors (TALEs) provides an alternative approach to the design of site-specific nucleases. Natural TALEs are transcription factors used by plant pathogens in the bacterial genus Xanthomonas. The DNA-binding domain of TALEs is unusual. The proteins, TAL effectors, have nuclear localization signals and an acidic transcription-activation domain. TALENs are novel fusion proteins that, like ZFNs, consist of assembled DNA-binding motifs coupled to FokI nuclease. The DNA-binding motifs of TAL effectors consist of a tandem repeat of typically 34 amino acids. Each repeat appears to bind to a single base pair based on a simple cipher. Each individual domain determines the specificity of binding to one DNA base pair in the TALE recognition sequence, and therefore arrays of four different repeat units are sufficient to generate TALEs with novel DNA recognition sites. Artificial TAL effectors targeted to novel sequences could activate transcription, thereby opening the door to a variety of TAL effector-based genome engineering applications.

The activities of custom-designed TALENs in human cells have efficiencies of NHEJ-induced mutagenesis ranging up to 45% of transfected cells. Sequence-specific DNA-binding proteins with predicted binding specificities have been generated in a matter of days, using molecular biology methods practiced by most laboratories. The activities of custom-designed TALENs in human cells have efficiencies of NHEJ-induced mutagenesis ranging up to 45% of transfected cells. This method was tested in iPS and showed that TALE nuclease mediated site-specific genetic modification with similar precision and efficiency as Zn finger nuclease.

### RNA Interference (RNAi) Technology.

RNA interference involves micro-(mi-) RNA and small interfering (si) RNA, which, upon base-pairing to their target sequence in a certain mRNA, cause degradation or prevent translation of the mRNA. This method may be useful to suppress the expression of a toxic mutant allele that causes the symptoms of a certain genetic disease. However, this method does not repair the underlying genetic aberration. Therefore, to suppress the expression of the mutant allele in a genetically abnormal iPSC, a method to continuously deliver the interfering RNA is needed. Various expression systems for either miRNA or siRNA have been developed using various vectors and promoters. The expression system for siRNA involves the formation of short hairpin (sh) RNA before the formation of a double-strand functional siRNA, while that for miRNA involves the formation of primary miRNA transcripts, followed by the formation of pre-miRNA, and finally a functional mature miRNA.

### Exon Skipping Technology.

Exon skipping technology causes deletion of selected exon(s) by targeting a sequence in the adjacent intron using an antisense oligonucleotide. This method can be used in genetic aberration where there is a mutation that causes a frameshift or a stop in the mRNA, and deletion of one/several frame-shifted exon(s) leads to a shorter, but still functional protein. However, the use of exogenous antisense oligonucleotide to cause exon skipping in iPSC needs continuous supply of the antisense oligonucleotide. Therefore, to repair a genetic aberration in iPSC, an expression vector needs to be engineered.

### Gene Transfer Method.

Gene transfer method may be useful in genetic diseases where there is genetic aberration that causes the absence of expression of a certain gene, such as in β thalassemia.

Type of Cell to be Transplanted and Integration into the Target Site.

The option is whether to use fully or partly differentiated iPPSCs or hematopoietic progenitor cells. Transplantation of partly differentiated iPSC or hematopoietic progenitor cells is intended to resume the differentiation in vivo into the mature desired cells.

Most of the methods of genetic repair, which may be used to repair patient-derived iPSC, use viral vectors as expression vectors, such as lentiviral-based vector in gene transfer technology, TALE nuclease genetic editing, RNAi technology, or AAV-based vector in RNAi technology and exon skipping technology. Clinical safety of these vectors can be achieved by deletion of promoter element in the viral long terminal repeat (LTR), termed self-inactivating (SIN) LTR, which may significantly decrease cellular transformation in vitro. Another approach to reduce oncogenesis is by insertion of an insulator element into the LTR.

Reprogramming of Tail Tip Fibroblasts from SS Mice to Embryonic Stem Cells with Lentiviral Vectors Encoding Transcription Factors

This representative method is from Chang C W et al., Stem Cells 27:1042-1049 (2009). Adult skin fibroblasts from a humanized mouse model of sickle cell disease are reprogrammed reliably by lentivirus to iPS cells by transduction with a polycistronic lentiviral vector encoding mouse cDNAs for Oct4, Sox2, Klf4, and c-Myc. and the reprogramming sequences can be efficiently deleted from the iPS cell genome.

### Production of OSK Polycistronic Lentiviral Vector

Briefly, human Oct4 cDNA (Clone 40125986; Open Biosystems, Huntsville, Ala.) is PCR amplified and modified with primers OCT4-F and OCT4-R to contain Not I and Swa I restriction sites at the 5′ end and a Kozak consensus sequence. At the 3′ end, the Oct4 stop codon was eliminated and replaced with nucleotides (nt) from PTV1 2A that will form a 22-nt overlap with the 5′ end of the Sox2 amplicon. Human Sox2 cDNA (Clone 2823424; Open Biosystems) is PCR amplified and modified with primers SOX2-F and SOX2-R to overlap with the 3′ end of the Oct4 amplicon and to append 2A nt sequences upstream of the Sox2 ATG. At the 30 end, the Sox2 stop codon is eliminated and replaced with nt from PTV1 2A that will form a 22-nt overlap with the 5′ end of the Klf4 amplicon. Human Klf4 cDNA (Clone 5111134; Open Biosystems) is PCR amplified and modified with primers KLF4-F and KLF4-R to overlap with the 3′ end of the Sox2amplicon and to append 2A nt sequences upstream of the Klf4 ATG. At the 30 end, the Klf4 stop codon is retained and Swa I and Sal I restriction sites were added. After PCR the individual amplicons are gel purified and used in a three-element fusion PCR at a 1:100:1 (Oct4/Sox2/Klf4) molar ratio along with primers OCT4-F and KLF4-R to produce a 3,623-bp amplicon containing the polycistron. The polycistron is gel purified and cloned into the general cloning vector pKP114 using the NotI and SalI restriction sites (enzymes from Roche Diagnostics, Basel, Switzerland) to produce pKP330 and sequenced for authenticity. Subsequently, the polycistron was removed from pKP330 as a SwaI (Roche Diagnostics) fragment and subcloned into a SwaI site downstream of the elongation factor 1 alpha (EF-1a) promoter in the lentiviral vector pDL171 to produce the OSK polycistronic lentiviral vector pKP332, which is also sequenced for authenticity. By the same strategy a second polycistronic lentival vector, pKP333, is produced that substitutes the PTV 1 2A peptide between Sox2 and Klf4 with the Thosea asigna virus 18 amino acid 2A-like sequence: GSG (linker) EGRGSLLTCGDVEENPGP (SEQ ID NO:5).

### Cell Culture and Viral Infections

Embryonic stem (ES) cells and iPS cells were cultured on irradiated MEFs in ES cell media consisting of Dulbecco's modified Eagle's medium supplemented with 1× nonessential amino acids, 1× penicillin-streptomycin, 1×L-glutamine (all from Mediatech, Manassas, Va.), 1× nucleosides (Chemicon, Temecula, Calif.), 15% fetal bovine serum (FBS; HyClone, Logan, Utah), 2-mercaptoethanol (Sigma-Aldrich, St. Louis, Mo.), and leukemia inhibitory factor (laboratory preparation). For preparation of lentivirus, 140 μg of the polycistronic vector (pKP332), 70 μg of the envelope plasmid (pMDG), and 105 μg of the packaging plasmid (pCMBVdR8.9.1) were cotransfected into 1.7×10⁷ 293 T cells by the CaCl₂ method as previously described. Virus-containing supernatant was collected 2 days after transfection, passed through a 0.45-μm filter, and concentrated by centrifugation at 26,000 rpm for 90 minutes at 8° C. in an SW-28 rotor using a Beckman XL-100 ultracentrifuge.

For iPPS cell induction, 3×10⁵ adult 12-week-old hbS/hbS male mouse tail-tip fibroblasts (TTFs) are seeded onto one well of a six-well plate. The next day, 2.5 μl of the concentrated virus is mixed with 2 ml of ES cell medium containing 8 μg/ml polybrene and added to the TTFs. Forty eight hours later, the TTFs are trypsinized and transferred to a 100-mm dish without MEFs and continuously cultured on the same dish for 3 weeks with daily media changes. Potential iPS cell colonies started to appear after 2-3 weeks. These colonies are individually picked and expanded on MEFs for analysis. To remove the integrated lentiviral and polycistronic sequences, iPS cells are either electroporated with a Cre-expressing plasmid (pCAGGS-Cre) or infected with a Cre-expressing adenovirus (rAd-Cre-IE). Individual colonies are picked and Cre-mediated removal of floxed sequences is verified by PCR and Southern blot analysis.

For the construction of rAd-Cre-IE (rAd-Cre-IRES-EGFP), Cre cDNA is PCR amplified from pCAGGS-Cre and inserted between the NheI and EcoRI sites of the expression vector pECIE, which contains an IRES-EGFP downstream of the MCS. The Cre-IE expression cassette is flanked by attL1 and attL2 sites, thus allowing transfer of the Cre-IE sequence from pEC-IE to pAd/p1-DEST (Invitrogen, Carlsbad, Calif.) by the LR reaction. The recombinant adenovirus is packaged in 293A cells according to the manufacturer's instructions. With the exception of the pKP332 construction, all of the PCRs performed in this study used ExTaq polymerase (Takara).

The reprogrammed embryonic stem cells are transfected with the lentiviral vector encoding the SS hemoglobin gene with the coding region of SEB gene substituted for the beta globin coding region by methods described above.

In Vitro Differentiation of the Embryonic, SEB Transfected, SS Hematopoietic Cells into Mature SS Erythrocytes Producing SEB

This representative method is from Shen J. Qu C K. In Vitro Hematopoietic Differentiation of Murine Embryonic Stem Cells in Methods in Molecular Biology, vol. 430: pp. 103-118 Hematopoietic Stem Cell Protocols Edited by: K. D. Bunting © Humana Press, Totowa, N.J.

### In Vitro Differentiation of ES Cells

1. Differentiation medium (the methylcellulose differentiation media contains the same reagents as liquid differentiation media, except that methylcellulose is added to 1% of the final volume):
a. Iscove's Modified Dulbecco's Medium (IMDM) (Invitrogen, Cat. No. 12440-053).
b. 15% FBS (Hyclone, regular FBS).
c. 4.5×10−4 M α-Monothioglycerol (MTG, Sigma, Cat. No. M-6145).
d. 2 mM 1-Glutamine (see above).
e. 50 μg/ml Ascorbic acid. Dissolve ascorbic acid at 5 mg/ml in H₂O and filter through 0.22 μm sterilization filter.
f. 200 μg/ml Human transferrin (Sigma, Cat. No. T8158).
g. 1% Methylcellulose (2× stock, Fluka, Cat. No. 64630). 2. ES-IMDM: 15% FBS (ES cell serum, see above), 1000 U/ml LIF, and 1.5×10⁻⁴ M of MTG in IMDM.
3. Gelatin: Prepare a 0.1% solution of gelatin in H2O, dissolve, and sterilize by autoclaving.
4. Methylcellulose-based feed medium:
a) ½ (v/v) primary differentiation medium (from above or freshly prepared).
b) 15% regular FBS.

### c) 1.5×10⁻⁴M of MTG

d) 150 ng/ml murine Kit Ligand/Stem Cell Factor (mSCF) (R&D systems, Minneapolis, Minn., USA, Cat. No. 455-MC)
e) 30 ng/ml murine Interleukin-3 (mIL-3) (R&D systems, Cat. No. 403-ML).
f) 20 ng/ml mouse Interleukin-6 (hIL-6) (R&D systems, Cat. No. 406-ML).
g) 3 U/ml human Erythropoietin (EPO)(R&D systems, Cat. No. 287-TC-500).
h) IMDM to the final volume.

5. 2× cellulose. Dissolve cellulose (Sigma, Cat. No. C-1794) in PBS at 2 U/ml. Filter sterilize through 0.45-nm filter.

6. Collagenase. Dissolve 1 g of collagenase (Sigma, Cat. No. C0310) in 320 ml PBS. After filter sterilization, add 80 ml of FBS. Aliquot and keep at −20° C.
7. Hemangioblast colony methylcellulose mixture:
a) 1% methylcellulose.

### b) 10% FBS.

c) SCF (100 ng/ml recombinant mSCF or 1% conditioned medium that was derived from medium conditioned by CHO cells transfected with mouse SCF expression vectors).
d) 25% D4T endothelial cell-conditioned medium (D4T endothelial cells are cultured in IMDM with 10% FBS. Remove media and change to 4% FBS in IMDM when it becomes 80% confluent. Culture additional 72 h and collect the supernatant. Spin down for 5 min at 228 g, Beckman GS-6, GH-3.8 rotor to remove the cell debris and filter sterilize the supernatants by 0.45-μm filter. Make 5- to 10-ml aliquots and keep at −80° C. Once thawed, it is kept at 4° C. for about 1 week).
e) 5 ng/ml mouse VEGF (R&D systems, Cat. No. 494-VE).
f) 10 ng/ml human IL-6.
g) IMDM to a final volume.
8. Hemangioblast expansion Medium:

### a) 10% FBS.

b) 10% horse serum (Invitrogen, Cat. No. 26050-088).
c) 5 ng/ml mouse VEGF.
d) 10 ng/ml mouse insulin-like growth factor-1 (IGF-1) (R&D systems, Cat. No. 791-MG).
e) 2 U/ml human EPO.
f) 10 ng/ml mouse basic fibroblast growth factor (bFGF) (R&D systems, Cat. No. 3139-FB).
g) 50 ng/ml mouse IL-11 (R&D systems, Cat. No. 418-ML).
h) 100 ng/ml SCF.
i) IL-3 [30 ng/ml recombinant murine IL-3 or 1% conditioned medium obtained from medium conditioned by X63 AG8-653 myeloma cells transfected with a vector expressing IL-3 (20)].

### j) 1% 1-Glutamine.

### k) 4.5×10−4 M of MTG.

l) IMDM to a final volume.
9. Matrigel-coated wells (the stock bottle of Matrigel should be thawed slowly on ice, diluted 1:1 populations is carried out in microtiter wells pretreated with a thin layer of Matrigel. The wells are coated by first spreading 5 μl of diluted Matrigel over the surface with an Eppendorf pipette tip. The plate should be kept on ice during this procedure. When the required number of wells has been coated, incubate the plate on ice for 10-15 min. Following this incubation, remove excess Matrigel from each well and then incubate at 37° C. for an additional 15 min before use.
11. Hematopoietic differentiation medium:
a) 1% methylcellulose.
b) 10% plasma-derived serum (Antech, Inc., Colorado, USA, Tyler, Tex., USA).
c) 5% protein-free hybridoma medium (PFHM-II; Invitrogen, Cat. No. 12040-077).
d) SCF (100 ng/ml mSCF or 1% conditioned medium).
e) 5 ng/ml mouse thrombopoietin (R& D System, Cat. No. 488-TO).
f) 2 U/ml human EPO.
g) 25 ng/ml mouse IL-11.
h) IL-3 (30 ng/ml recombinant mIL-3 or 1% conditioned medium).
i) 30 ng/ml mouse granulocyte-macrophage colony-stimulating factor (GMCSF) (R&D systems, Cat. No. 415-ML).
j) 30 ng/ml mouse G-CSF (R&D systems, Cat. No. 414-CS).
k) 5 ng/ml mouse M-CSF (R&D systems, Cat. No. 416-ML).
l) 5 ng/ml mouse IL-6.

m) IMDM to the final volume.

### In Vitro Hematopoietic Differentiation of Murine ES Cells:

### Primary Differentiation Step, Formation of EBs

Two different culture methods usually have been used to promote hematopoietic differentiation: 1) Methylcellulose-based semisolid media, a highly viscous media that does not encourage cellular migration or aggregation once seeded; 2) Liquid suspension culture, where cells are free to aggregate and move within the culture media.

### Methylcellulose-Based Semisolid Culture

1. Two days before setting up differentiation, split ES cells (4×10⁵ ES cells per 60-mm dish) into E S-IMDM medium without feeder cells in the dishes. An plates should be gelatinized.
2. Change the medium the next day.
3. Aspirate the medium from the dishes.
4. Add 1 ml of trypsin-EDTA, swirl, and remove quickly.
5. Add 1 ml trypsin and wait until cells start to come off. It usually takes about 1-2 min
6. Stop the reaction by adding 1 ml FBS and 4 ml IMDM and pipette up and down to make single cell suspension.

### 7. Centrifuge for 5-10 min at 228 g.

8. Wash the cell pellet in 10 ml IMDM (without FBS).
9. Resuspend the cell pellet in 5 ml IMDM (with 10% FBS), count viable ES cells.
10. For differentiation as follows: add 6,000-10,000 ES cells per milliliter of methylcellulose differentiation media to obtain day 2.75-3 EBs. Add 4,000-5,000 cells per milliliter to obtain day 4-5 EBs. Add 500-2,000 cells per milliliter to obtain day 6-10 EBs.
11. Place dishes into a larger covered Petri dish along with an open 35-mm Petri dish containing 3 ml of sterile water and incubate at 37° C. in a 5% CO₂ and moisture-saturated incubator until further analysis is performed.

### Suspension Culture

1. Follow steps 1-10 as described for Methylcellulose-based semisolid culture.
2. Plate into low-adherence Petri dishes at 4×10⁵ cells per dish. Small aggregates (simple EBs) will be visible in 24 h. These simple EBs can be transferred into methylcellulose between 24 and 48 h.
3. If continuing in the liquid culture system, the media must be changed every 3-4 days. The EBs will tend to aggregate into clumps with regions of necrosis. To avoid this, break clumps apart by using a large mouth pipette (25 ml) such that you do not disrupt the EBs themselves. Transfer the EBs to a tube and allow them to sink to the bottom. Carefully aspirate off the old media, replace with fresh medium, and replate into the Petri dish.

### Harvest of ES Cell-Derived Embryoid Bodies (EBs)

1. For EBs in liquid: transfer media containing EBs into 50-ml tubes. Wash the plate with IMDM and incubate at room temperature for 10-20 min. EBs settle to the bottom of the tube. For EBs in methylcellulose: add equal volume of cellulose (2 U/ml, final 1 U/ml) and incubate 20 min at 37° C. Collect EBs in 50-ml tubes. Wash the plate with IMDM and add to the tube to ensure all EBs are collected. Incubate at room temperature for about 10-20 min and EBs will settle down to the bottom of the tube.
2. Aspirate off media, add Trypsin-EDTA, or collagenase.
a) For EBs up to 8 days old, add 2-3 ml Trypsin-EDTA and incubate for 2-3 min at 37° C. Add IMDM containing 5% FBS to neutralize trypsin. Disrupt EBs by passing through a 20-G needle on a 3-cc syringe three times.
b) For EBs 9 or more days old, add 2-3 ml of collagenase and incubate at 37° C. for 1 h, swirling gently following 30 min of incubation. Ensure the EBs remain in solution and are not on walls of tubes. Add IMDM containing 5% FBS to neutralize collagenase. Disrupt EBs by passing through a 20-G needle on a 3-cc syringe three times as above.
3. Transfer to a 14-ml tube and pellet cells by centrifugation at 350 g for 5-8 min
4. Remove supernatant and resuspend the cells in a minimum volume of IMDM with 2% FBS.
5. Count the viable cells.

### Second Differentiation Step, Clonal Assays of EBs

In the presence of vascular endothelial growth factor (VEGF) in methylcellulose cultures, these EB-derived precursors generate blast cell colonies that display hematopoietic and endothelial potential. These progenitors or BL-CFC are present transiently within the EBs for approximately 36 h, between day 2.5 and 4 of differentiation, preceding the onset of primitive erythropoiesis. The BL-CFC represents the in vitro equivalent of the hemangioblast and the earliest stage of hematopoietic. Beyond day 4 of differentiation, the number of BL-CFC declines with the commitment to the hematopoietic program with the appearance of significant numbers of primitive erythroid progenitors. Hematopoietic progenitors present within EBs are successfully analyzed by flow cytometry and by direct replating EB cells into methylcellulose cultures to measure the frequency of hematopoietic progenitors. Additionally, EB cells are sorted for early hematopoietic markers to further analyze their hematopoietic cell potential.

### Hemangioblast Stage

Most BL-CFC express Flk1, and a subpopulation of Flk1+ cells also expresses the transcription factor Sc1. The lack of significant Flk1 expression indicates that the population has not yet progressed to the hemangioblast stage of development. Thus BL-CFC can be initially screened by levels of expression of the receptor Flk1 or other marker gene colonies. Blast colonies develop within 3-4 days of culture and are recognized as clusters of cells readily distinguished from secondary EBs developing from residual undifferentiated ES cells. Blast colonies and secondary EBs are the predominant type of colonies present in these cultures.

1. Add 3-6×10⁴ EB cells per milliliter of hemangioblast colony methylcellulose mixture. Add 1 ml of the mixture into each of 35-mm Petri dishes.
2. Gently swirl the dishes to disperse the mixture evenly.
3. Place dishes into a larger covered Petri dish along with an open 35-mm Petri dish containing 3 ml of sterile water and incubate at 37° C. in a humidified 5% CO₂ incubator for 3-4 days.
4. For FACS analysis, antibody staining is carried out as follows: Cells are collected and resuspended in 100 μl of PBS containing 10% FBS and 0.02% sodium azide. An appropriate amount of antibody is added, and the cells are incubated on ice for 20 min Following the staining step, the cells are washed two times with the same media and then resuspended in 300 μl of staining buffer, then transferred to a 5-ml polypropylene tube for analysis.
5. To analyze the hematopoietic of the blast colonies, individual colonies are picked from the methylcellulose and cultured further in hemangioblast expansion medium on matrigel-coated microtiter wells. After 4 days of growth, the non-adherent cells of each well are harvested and assayed for hematopoietic progenitor potential in 1 ml hematopoietic differentiation medium used for the growth of hematopoietic precursors.

### Hematopoietic Stage

Shortly after the peak of the hemangioblast stage of development, committed hematopoietic progenitors are detected within the EBs by morphology or cytological staining. When EB cells are directly replated, day 5-6 EBs are typically used for a primitive erythroid colony, and day 7-10 EBs for definitive erythroid progenitor analysis. The following is the protocol for direct EB replating.

1. Prepare methylcellulose-based hematopoietic differentiation medium.
2. Add 0.3 ml of cells at 1-5×10⁵ per milliliter to each tube containing the 3-ml hematopoietic differentiation medium and vortex thoroughly and allow to stand 3-5 min
3. Plate 1.1 ml of the cell suspension per 35-mm low-adherence Petri dish.
4. Place dishes into a larger covered Petri dish along with an open 35-mm Petri dish containing 3 ml of sterile water and incubate at 37° C. in a humidified 5% CO₂ incubator.
5. Primitive erythroid colonies are scored at day 5-6 of culture, whereas definitive erythroid, macrophage, and multilineage colonies are counted after 7-10 days of culture.

Globin genes are isolated from any of the number of clones containing portions of the β-globin locus of humans are widely available in the art. Globin genes from patients suffering from hemoglobinopathies may be cloned by preparing a genomic library from DNA harvested from the patient (for example, from leukocytes) using methods known in the art and then using globin gene probes derived from cloned genes of the normal globin locus (which are widely available) to identify, by hybridization, genomic clones containing globin gene sequences (Benton and Davis Science 196:180 (1977); Grunstein and Hogness, Proc. Natl. Acad. Sci. 72:3961-3965 (1975)). Genomic clones identified in this manner may then be analyzed by restriction mapping and sequencing techniques to potentially identify genes bearing mutations.

DNase I hypersensitivity sites associated with the β-globin gene locus may be used according to the invention to direct the expression of any gene of interest in erythroid cells. DNase I HS sites derived from human β-globin genes may be used, as may any DNase I HS site from any erythroid specific gene whatsoever, provided that the DNase I HS site in question results in substantial transcription of the gene in question in erythroid cells. According to the invention, β-globin DNase I HS sites HIS, HS II, HS III, HSIV, HSV or HSVI. Any combination thereof, or any duplication thereof, may be used according to the invention; it appears however, that a single copy of HS I may not be sufficient to effectively boost transcription.

Globin DNase I HS sites may be isolated from any of the cloned regions of the globin clusters widely available to those in the art, or alternatively, from the following recombinant DNA molecules described herein including, HS I-V-β and HSI-V which have been deposited with the American Type Culture Collection (ATCC) and assigned the accession numbers 40666 and 40664 respectively. In addition, DNase I HS sites that have been mapped may be obtained using any clone of the globin locus and then using the standard technique of chromosome walking to reach a previously identified DNase I HS site. In addition, new erythroid-specific DNase I hypersensitivity sites may be identified by sensitivity to DNase I digestion and utilized according to the invention.

The extent of expression of the tumoricidal molecule can be controlled by altering the number of DNase I HS sites in the recombinant constructs of the invention. In general, the greater the number of HS sites included, the higher the level of expression of the gene of interest that will result.

The tumoricidal transgene and at least one β-globin HS site may be inserted into a cloning vector which can be used to transfect and transform appropriate host cells so that many copies of the gene sequences are generated. This can be accomplished by ligating the DNA fragment into a cloning vector which has complementary cohesive termini. However, if the complementary restriction sites used to fragment the DNA are not present in the cloning vector, the ends of the DNA molecules may be enzymatically modified. It may prove advantageous to incorporate restriction endonuclease cleavage sites into the oligonucleotide primers used in polymerase chain reaction to facilitate insertion into vectors. Any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and gene of interest may be modified by homopolymeric tailing. In addition, in particular embodiments of the invention the recombinant nucleic acid molecule of the invention may be inserted into any viral vector capable of infecting erythroid cells, including but not limited to lentiviral vectors, retroviruses and Friend Virus A provided that the dominant element controlling transcription of the gene of interest is the erythroid-specific HS site and the β-globin promoter/enhancer.

Provided an erythroid-specific DNase I HS site is included, the recombinant nucleic acid vectors of the invention may include any transcriptional promoter known in the art, including but not limited to the SV40 early promoter region (Bernoist and Chambon Nature 290:304-310 (1981), the promoter contained in the 3′ long terminal repeat of Rous sarcoma virus (Yamamoto. et al., Cell 22:787-797 (1980), the herpes virus thymidine kinase promoter (Wagner et al., Proc. Natl Acad Sci 78:144-1445 (1981)). The regulatory sequences of the metallothionine gene (Brinster et al., Nature 296:39-42 (1982) and the following animal transcriptional control regions, which exhibit tissue specificity and have been utilized in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald Hepatology 7:425-515 (1987); insulin gene control region which is active in pancreatic beta cells (Hanahan Nature 315:115-122 (1985). Immunoglobulin gene control region which is active in lymphoid cells (Grosschedl et al., Cell 38:647-658 (1984); Adames et al., Nature 318:533-538 (1985); Alexander et al., Mol Cell Biol. 7:1436-1444(1987), mouse mammary tumor virus control region which is active in testicular, breast, lymphoid and mast cells (Leder et al., Cell 45:485-95(1986), albumin gene control region which is active in liver (Pinkert et al., Genes Dev 1:268-276 (1987), alpha-fetoprotein gene control region which is active in liver (Krumlauf et al., Mol Cell Biol 5:1639-1648 (1985); Hammer et al., Science 235:53-58(1987), alpha 1-antitrypsin gene control region which is active in the liver (Kelsey et al., Gene Dev 1:161-171 (1987), beta-globin gene control region which is active in myeloid cells (Mogram et al., Nature 315:338-340 (1985); Kollias et al., Cell 46:89-94 (1986), myelin basic protein gene control region which is active in oligodendrocyte cells in the brain (Readhead et al., Cell 48:703-712 (1987), myosin light chain-2 gene control region which is active in skeletal muscle (Sani et al., Nature 314:283-286 (1985) and gonadotropic releasing hormone gene control region which is active in the hypothalamus (Mason et al., Science 234:1372-1378 (1986)).

Transfection of cell lines may be performed by the DEA dextran method (McCutchen & Pagano J Natl Cancer Inst 41:351-357 (1968)), the calcium phosphate procedure (Graham et al., J Virol 33:739-748 (1973)) or by any other method known in the art including but not limited to microinjection, lipofection and electroporation.

The recombinant molecules of the invention may be used to induce expression of the tumoricidal transgene in erythroid cells due to the presence of the erythroid-specific DNase I HS sites. In a preferred embodiment of the invention, the recombinant molecules of the invention may be used to produce a tumoricidal transgene product in the erythroid cells of a human or non-human animal. According to the present invention, erythroid cells may be harvested and the tumoricidal protein product may be harvested and identified by lysing the cells and purifying the protein product using methods known in the art including but not limited to chromatography (e.g., ion exchange affinity, and sizing column chromatography, centrifugation differential solubility, electrophoresis, or any other standard technique for the purification of proteins). It may be necessary to transform the cells, using any method known in the art while the cells are in a relatively undifferentiated state, and then to induce the cells to differentiate and subsequently produce the tumoricidal protein. For example, and not by way of limitation, the recombinant molecules of the invention may be transfected into erythroid cells which may then be induced to differentiate (e.g. using dimethylsulfoxide).

These mature SS cells or SS progenitor cells are tested in murine models provided in the section below on “Murine Models.” They are also tested in humans furnished in Example 3.

Use of the SS Progenitor Cells or Normal Hematopoietic Progenitor Cells Transduced with SS Genes and Tumoricidal Transgenes for Autologous or Allogeneic Transplantation in Humans

The present invention contemplates the source of the genetically engineered SS cells for administration to patients with cancer can be autologous, allogeneic or from cord blood. Preferably the progenitor cells are autologous and genetically engineered to express the SS phenotype in vitro as herein described. In all cases, CD34+, SCA+ hematopoietic progenitors of the erythroid lineage are used. This will prevent a the occurrence of graft versus host disease in the recipients and obviates the need for the use of immunosuppression related thereto. In all cases, donor and recipient CD34+, SCA+ hematopoietic progenitor cells must be HLA matched and mature erythrocytes similarly ABO matched. Mortality is considerably lower with autotransplantation than with allotransplantation

### Autologous Hematopoietic Cell Transfer:

In the present invention, the preferred source of hematopoietic cell transplant is autologous CD34+, SCA+ hematopoietic progenitor cells. These cells can be obtained directly from the bone marrow of the recipient or from the peripheral blood after short induction period. Bone marrow is obtained by repeated aspiration of the posterior iliac crests while the donor is under general or local anesthesia was the first source of hematopoietic stem cells. Discomfort from the harvesting procedure usually disappears within two weeks, and serious effects are rare (two deaths in 8000 collections). Because marrow stem cells detach continuously, enter the circulation, and return to the marrow, peripheral blood is a convenient source of hematopoietic stem cells and has replaced marrow for autologous and most allogeneic transplantations. Induction treatment consists of 3 to 6 cycles of chemotherapy or other anticancer treatment in order to reduce the tumor burden. Stem-cell mobilization from the bone marrow enhanced by using granulocyte colony-stimulating factor (G-CSF), either alone or with cyclophosphamide. The combination of G-CSF and AMD3100—a small-molecule reversible inhibitor of CXC chemokine receptor 4 (CXCR4), a receptor on CD34+ cells that mediates signals to potentiate the adhesion of CD34+ cells to marrow—is superior to G-CSF alone in mobilizing CD34+ cells. Leukapheresis in an adult, performed through the antecubital veins, can process up to 25 liters of blood in four hours, which usually yields enough CD34+ peripheral-blood stem cells to ensure rapid engraftment. After collection, stem cells are usually precryopreserved in dimethyl sulfoxide at temperatures below −120° C. The cells are transfected with nucleic acids encoding SS hemoglobin as described above and expanded in vitro to obtain sufficient numbers for autologous transplantation as described above. The minimal dose of CD34+ cells necessary for safe engraftment is 2×10⁶ per kilogram of body weight. The hematopoietic stem cells are infused 48 hours after melphalan administration through a central venous catheter at a rate ranging from 5 to 20 ml per minute. The patient may be premedicated with an antihistamine, an antiemetic agent, an antipyretic agent, and corticosteroids to mitigate infusion reactions. The infusion is usually performed in a reverse-isolation room. The infusion of autologous stem cells itself is associated with a variety of adverse effects, including nausea and vomiting, headache, and chills and fever. These effects are caused by the release of cytokines from lysed cellular elements in the stem-cell infusate and by the effects of dimethyl sulfoxide used as the cryopreservative. In most patients, such effects are mild and transient, although anaphylaxis and cardiac arrest have been reported infrequently. Blood counts are monitored weekly.

### Allogeneic and Cord Hematopoietic SS Cell Progenitor Transfer

These methods involve ABO and DNA typing to identify HLA alleles and the most closely matched donor, since the use of a closely matched donor increases the chances of successful engraftment and reduces the risk of GVHD. The number of matched unrelated donors is limited by the extensive polymorphism of HLA genes. Collection of the hematopoietic progenitor cells, storage and transfection with SS genes is identical to that for autologous transplantation.

If transplantation is urgent or if suitable donors are not found, cord blood can be used. Blood from the umbilical cord and the placenta is rich in hematopoietic stem cells but limited in volume. It is collected immediately after birth and then frozen. The transplantation of cord blood requires less-stringent HLA matching than the transplantation of adult peripheral blood or marrow, because

mismatched cord-blood cells are less likely to cause GVHD. The results are better with fewer HLA mismatches and greater numbers of CD34+ cells. The use of additional grafts from different donors may improve engraftment, particularly when the first graft contains few cells. Cord-blood stem cells can be transfected and expanded in vitro as described above for autotransplantation. The less-stringent HLA requirements for cord-blood transplantation would permit a smaller donor pool to serve virtually all potential recipients.

### Myeloablative Conditioning

Marrow-ablative doses of chemotherapy and total-body irradiation (TBI) were thought to be necessary to eradicate the underlying malignancy, to provide immunosuppression to the recipient, which would allow engraftment of donor hematopoietic cells and to create ‘marrow space’. High-dose regimens are still used for a large number of patients, particularly those with aggressive malignant diseases where there is a need for strong anti-leukemia or anti-tumor effect. The combinations of cyclophosphamide and total body irradiation or busulfan are established myeloablative regimens but other combinations, such as busulfan and fludarabine are being used increasingly. While this approach has been successful in providing long-term survival for some patients with otherwise incurable diseases, its usage has been restricted to patients younger than 50-55 years of age owing to associated toxicity and high nonrelapse mortality rates. Patients with pre-existing comorbidities, or those who underwent previous extensive chemotherapy or radiation treatment are particularly high risk for developing serious, potentially fatal regimen-related toxicities, such as sinusoidal obstruction syndrome (also known as veno-occlusive disease of the liver) or idiopathic interstitial pneumonia.

### Reduced Intensity & Nonmyeloablative Regimens

To prevent graft rejection and increase pretransplantation host T-cell immunosuppression, administration of fludarabine at 30 mg/m2 is used together with of 2 Gy TBI at a rate of 0.07 to 0.20 Gy/min from linear accelerators or opposing dual cobalt-60 sources on day 0. Postgrafting immunosuppression consisted of Postgrafting immunosuppression consists of cyclosporine or tacrolimus combined with mycophenolate mofetil. This nonmyeloablative conditioning regimen is associated with shorter in-patient hospital stays, reduced need for transfusions and a shorter duration of neutropenia with fewer bacterial infections.

### Transplantation Involving a Haploidentical Donor

The transplantation of stem cells from a parent, sibling, or child of a patient with only one identical HLA haplotype was initially associated with high rates of engraftment failure and GVHD—complications that predictably caused death soon after transplantation. In this type of transplantation, natural killer cells express combinations of activating and inhibitory killer-cell immunoglobulin-like receptors that interact with class I HLA epitopes. The balance of signals determines the cytolytic activity of the natural killer cells, a process that is inhibited by the recognition of self-epitopes by the immunoglobulin-like receptors. Alloreactivity improves the chances of engraftment and reduces the risk of GVHD.

Introduction of Suicide Genes into Genetically Engineered SS Hematopoietic Progenitor Cells

Should it be necessary to remove the hematopoietic SS cells from the host, a safety, or “suicide gene” is incorporated into the transferred hematopoietic stem cells. In this strategy, a prodrug that is administered in the event of an adverse event is activated by the suicide-gene product and kills the transduced cell. Expression of the gene encoding herpes simplex virus thymidine kinase (HSV-TK) has shown promise as a safety switch in patients receiving cellular therapies, but its mechanism of action requires interference with DNA synthesis so that cell killing may take several days and be incomplete, resulting in a prolonged delay in clinical benefit. Moreover, an antiviral prodrug (e.g., ganciclovir) is required for cell elimination. An alternative strategy that relies on inducible caspase proteins to exploit the mitochondrial apoptotic pathway. It uses an inducible hematopoietic cell safety switch that is based on the fusion of human caspase 9 to a modified human FK-binding protein, allowing conditional dimerization. When exposed to a synthetic dimerizing drug, the inducible caspase 9 (iCasp9) becomes activated and leads to the rapid death of cells expressing this construct. In this system inducible caspase 9 (iCasp9)—is used to tranfect allogeneic stem-cells. These cells can be killed by a single dose of a dimerizing drug.

### Generation of Allodepleted T Cells

T cells from the donors of these haploidentical stem cells were allodepleted before they were genetically modified to induce the expression of iCasp9 and the selectable marker ΔCD19.

For allodepletion, peripheral blood mononuclear cells (PBMCs) obtained from HLA-haploidentical stem-cell donors are cocultured with irradiated recipient Epstein-Barr virus (EBV)-transformed lymphoblastoid cell lines at a ratio of 40:1 responder (donor) cells to stimulator (recipient) cells in serum free medium (AIM V, Invitrogen). After 72 hours, activated T cells that expressed CD25 were depleted from the coculture by overnight incubation in an immunotoxin that was constructed by linking the monoclonal antibody RFT5 through a sterically hindered disulfide linker (SMPT) to deglycosylated ricin A-chain (dgA). Allodepletion was considered adequate if the residual CD3+CD25+ population was less than 1% and if residual proliferation in response to recipient cells by [3H]thymidine incorporation was less than 10%.

The transgene SFG.iCasp9.2A.ΔCD19 consists of iCasp9, which is linked through a sequence of 2A-derived nucleotides to truncated human CD19 (ΔCD19); iCasp9 consists of the sequence of the human FK506-binding protein (FKBP12; GenBank number, AH002818) with an F36V mutation, connected through a Ser-Gly-Gly-Gly-Ser linker to the gene encoding human caspase 9 (CASP9; GenBank number, NM001229), which is deleted for its endogenous caspase activation and recruitment domain. 17-20 FKBP12-F36V binds with high affinity to an otherwise bioinert small molecule dimerizing agent, AP1903, with truncated CD19 (ΔCD19) serving as a selectable marker. In the presence of the drug, the iCasp9 promolecule dimerizes and activates the intrinsic apoptotic pathway, leading to cell death. The safety and efficacy of the transgene have previously been tested in vitro and in small-animal models. 17 Detailed methods for T-cell activation and expansion and for retroviral transduction have been described previously.

### Infusion of Allodepleted T Cells

Patients in whom GVHD developed after the infusion of allodepleted T cells received 0.4 mg of the dimerizing agent AP1903 (Bellicum Pharmaceuticals) per kilogram as a 2-hour infusion, in accordance with pharmacokinetic data showing plasma concentrations of 10 to 1275 ng per milliliter in patients receiving doses ranging from 0.01 to 1.0 mg per kilogram, with plasma levels falling to 18% of the maximum half an hour after infusion and falling to 7% of the maximum 2 hours after infusion. At these concentrations of the dimerizing drug, preclinical studies showed little variation in the induction of apoptosis among patients, with consistent elimination of more than 90% of iCasp9-expressing cells.

### Complications

GVHD is the most important complication of allogeneic transplantation Acute GVHD damages the skin, gut, and liver. A pruritic micropapillary rash can affect the palms, soles, or face and may

become generalized. Nausea, vomiting, abdominal pain, diarrhea, bloody stool, and jaundice may occur. GVHD and its treatment with corticosteroids cause profound immunodeficiency, predisposing
the patient to fatal infection. The principal risk factor is HLA mismatch, but GVHD may occur despite an HLA-matched donor and the use of preventive measures. If prophylaxis is not provided, serious acute GVHD affects almost every recipient. A reduced-intensity regimen of total lymphoid irradiation and antithymocyte globulin may decrease the incidence of GVHD. Gene modification of donor T cells is a potential means of treating GVHD. Viral genes that are capable of converting drugs into lethal products have been expressed in donor T cells, which can then be eliminated if severe GVHD develops.

Mucositis is the most common complication of myeloablative preparative regimens. The second most common acute adverse effect is a potentially fatal syndrome of painful hepatomegaly, jaundice, and fluid retention, traditionally called hepatic veno-occlusive disease of “sinusoidal obstruction syndrome.” Transplantation-related lung injury occurs within four months after the procedure, and the mortality rate exceeds 60 percent. Transplantation-related infections result from damage to the mouth, gut, and skin from preparative regimens as well as from catheters, neutropenia, and immunodeficiency. Reduced-intensity regimens are associated with a lower rate of early infections than are myeloablative regimens, but the risk of late infection seems to be the same.

Prolonged neutropenia, GVHD, and the administration of corticosteroids predispose patients to

fungal infection, a life-threatening complication of allogeneic transplantation. Cytomegalovirus pneumonia has become rare with the use of techniques involving antigens or the polymerase chain reaction that detect subclinical cytomegalovirus infection and make early treatment possible.

Delayed complications, particularly chronic GVHD, often resembles scleroderma or Sjögren's syndrome. Chronic GVHD can cause bronchiolitis, keratoconjunctivitis sicca, esophageal stricture, malabsorption, cholestasis, hematocytopenia, and generalized immunosuppression. Treatment with corticosteroids may be needed for two years or longer. Corticosteroids can cause a variety of complications, including aseptic necrosis of bone and osteoporosis, and may predispose the patient to fatal infections. If severe hypogammaglobulinemia occurs, treatment with intravenous immune globulin can reduce infections. Most women fail to ovulate after undergoing transplantation. Hormonal suppression of the ovaries before the preparative regimen is administered might permit the recovery of ovulation after transplantation.

For children, growth and development are impaired by myeloablative preparative regimens, but growth hormone therapy can increase height in children who have undergone hematopoietic stem-cell transplantation. The frequency of secondary cancers is increased after transplantation. After allotransplantation, the incidence of cancers of the skin, oral mucosa, brain, thyroid, and bone is increased. Myelodysplasia and acute leukemia are complications of autologous transplantation for Hodgkins and non-Hodgkins lymphomas. The type and intensity of the pretransplantation chemotherapy used affect this risk.

Survivors of transplantation must avoid carcinogens, particularly tobacco. They should be followed indefinitely to detect early cancer or precursor lesions. They should also be observed for other conditions that have been reported: hypothyroidism, sexual problems, depression, and anxiety.

Use of the SS Hematopoietic Progenitor Cells Alone or Transduced with Tumoricidal Transgene for Autologous or Allogeneic Transplantation in Mice.

For autologous or allogeneic transplantation studies in mice bone marrows from SS mice are isolated from the femurs and tibias and SCA+ erythroid progenitor cells, separated by flow cytometry and transfected with tumoricidal transgenes SEB and SPEA as described above and resuspended at 1×10⁷ cells/mL in PBS. SCA+ donor bone marrow cells 2×10⁶ in 200 μL of PBS were injected via the femoral vein into recipient mice irradiated with two doses of 500-525 rad, 3 h apart. The chimeras were kept in autoclaved cages, with 0.2% neomycin sulfate (Sigma) in the drinking water for 2 weeks, after which sterile drinking water was used. They were used after a 3 month rest period to allow for full reconstitution.

Autologous and allogeneic SS cell transplant is carried out in mice using tumor models in the Tumor Models section below. They are also used to test tumor outgrowth in the SS mice models as described above and in humans as disclosed in Example 4.

### SS Cells, Erythroid Progenitors/Erythroblasts Transduced by Genomic Oncolytic Viral DNA Comprising SS β-Globin Promoter/Enhancer and LCR

The present invention contemplates infecting SS cells, erythroid progenitors/erythroblasts or SS erythroid pluripotent stem cells or erythroleukemia cells with the lentiviral vector described above containing genomic DNA of an oncolytic virus under control of the β-globin promoter/enhancer. The genomic viral DNA vector may retain its promoter or its promoter may be removed before subcloning into the lentiviral vector. The transduced erythroid cells containing the lentiviral vector synthesize the oncolytic virus are capable of differentiating into mature RBCs. Despite enucleation the latter cells are capable of sustaining oncolytic viral synthesis. Because these cells do not produce interferon, interferon-dependent oncolytic viruses replicate freely in the mature SS RBC.

In an example of this method, plasmid pVSV-XN2 (Lawson N D et al., Proc Natl Acad Sci 92:4477-4481(1995)) is used which contains the entire vesicular stomatitis virus (VSV) genome and gives rise to infectious VSV. It has unique XhoI site flanked by T7 bacteriophage promoter and the VSV N nucleic acids. It displays genes encoding the five proteins N, P, M, G, and L and the pBSSK+ vector sequence as well as regions generated by PCR. Transcription from the T7 promoter generates the complete positive-strand VSV RNA. Plasmid pVSV-XN2 with its T7 reverse transcriptase promoter of pVSV-XN2 is subcloned into the lentiviral vector substituting for the (3-globin coding region. This may be enhanced by digestion of pVSV-XN2 with XhoI. Transcription of infectious oncolytic/oncotropic recombinant (rVSV) is thus under control of the β-globin promoter/enhancer as described above. SS erythroblasts/progenitors or erythroleukemia cells are infected with this vector and differentiate in vitro into mature SS cells containing the VSV virus using methods described in the preceding section.

The mature SS cells, SS progenitors, erythroblasts or erythroleukemia cells (10⁵-10¹¹) producing the oncolytic virus are administered parenterally (preferably by injection or infusion intravenously or intraarterially) to tumor bearing hosts as described herein in the “Tumor Models” section and deposit in the tumor neovasculature where they undergoes hemolysis releasing oncolytic virus particles and/or tumoricidal molecules into the tumor parenchyma. DNA encoding any oncolytic virus or oncolytic viral fragment or viral homologue optionally containing a tumoricidal transgene is useful in this invention. Oncolytic viral genomes that are useful in the above method include but are not limited to measles virus (Schneider et al., J Virol 74:9928-36 (2000)); reovirus (Roner et al., Proc Natl Acad Sci 98: 8036-8041 (2001)); Sindbis virus (Strauss et al., J Virol 133:92-110 (1984)); Semliki forest virus (Kaariainen et al., J. Cell Sci Suppl. 7: 231-250 (1987)); Venezuelan equine encephalitis virus, (Kinney et al., Virology 191:569-580 (1992)); Newcastle disease virus (NCBI GenBank AF309418); poliovirus; (Racaniello et al., Proc. Natl. Acad. Sci. 78: 4887-4891 (1981)); herpes simplex virus (NCBI GenBank Z86099). Viral vectors for this purpose may be genomic viral DNA or RNA or a fragment of a homologue of viral DNA or RNA.

The above viral genome also incorporates nucleic acids encoding tumoricidal molecules and siRNAs. Tumoricidal transgenes including but not limited to IL-4, IFN-β, and TNFα are useful. siRNAs, shRNAs or miRNAs are also useful in silencing tumor cell oncogenes, or nucleic acids encoding cyclins, VEGF, hypoxia inducible elements, heme oxygenase, catalase, superoxide dismutase or IFN-β initiator genes, any anti-apoptotic, pro-angiogenesis, pro-proliferative or pro-metastatic molecule. Tumoricidal molecules and siRNAs are inserted between the G and L nucleic acids of the pVSV-XN2 vector (Obuchi M et al., J Virol 77: 8843-56 (2003); Fernandez M et al., J Virol 76:895-904 (2002)). Tumoricidal transgenes are amplified from pLEGFP-C1 (Clontech Laboratories, Palo Alto, Calif.) plasmids, by PCR. For IL-4 and IFN-β, the primers (SEQ ID NO:4-6) 5′GGCACTCGAGATGGGTCTCAACCCCCAGCTAGTTG and (SEQ ID NO:4-7) 5′GCCGTCTAGACTACGAGTAATCCATTTGCATGATGC are used (foreign gene is in bold). Substitution mutations in the M molecule of the VSV have been shown to inactivate the interferon activator in tumor cells. This construct is also useful in the present invention under control of the SS β-globin promoter/enhancer (Stojdl D F et al., Nat. Med. 6: 821-825 (2000); Lichty B D et al., Hum Gene Ther 15: 821-831 (2004)). Indeed SS β-globin promoter/enhancer promoter is useful with any alteration of the VSV viral genome that improves viral oncotropic and/or oncolytic activity.

SS Erythroid Progenitor Cells as Vehicles for Transfer of Tumoricidal Transgenes, Viruses/Oncolytic Virus Alone or Virus/Oncolytic Virus or Vectors Incorporating Regulatory RNAs, siRNA, shRNA, miRNA, Activating RNAs.

Systemic delivery of naked or conjugated siRNA, shRNA or miRNA to its tumor target is generally unsuccessful for several reasons. First, the size of unconjugated therapeutic RNAs are 7-20 kDa and molecules less than 50 kDa are filtered/excreted by the kidneys. Second, transfer of therapeutic RNA from the blood to the target tissue is interdicted because molecules larger than 5 nm diameter such as therapeutically complexed RNA, cannot cross the capillary endothelium. Third, delivery into the bloodstream is inhibited by phagocytic immune cells, such as macrophages and monocytes, which remove complexed RNAs from the body.

The present invention contemplates the use of SS erythroid progenitor cells (SSEPCs) and SSiPPSCs transduced with tumoricidal transgenes, oncolytic virus or genome, regulatory RNAs, i.e. siRNA, shRNA, RNA motors, micro RNA, gene activating RNA (collectively TTORs) to produce a tumor killing. Oncolytic viruses or genomes that incorporate tumoricidal transgenes are also useful in this invention. Examples of tumoricidal transgenes, oncolytic viruses and RNA nucleotides useful in this invention that can be inserted, packaged and protected in SSEPCs or SSiPPSCs directly or incorporated into the β-globin lentiviral vector are given in Table 1. Such TTORs can be expressed in both SSEPCs and enucleated mature SSRBCs differentiating from the SSEPCs. These mature SSRBCs can simultaneously produce SS hemoglobin along with the TTORs and their gene protein. Such TTORs include but are not limited to granzymes, perforin, granulysin, tumoricidal cytokines, IFNγ, IFNα, IFNβ, TNFα, IL-12, TRAIL FADD, complement membrane attack complex, chemokines, bacterial toxins such as superantigens, pseudomonas exotoxin A, diptheria toxin, pertussis toxin, ricin toxin, holins, used alone or conjugated biochemically or recombinantly to tumor specific targeting agents. Examples of useful oncolytic viruses include but are not limited to adenovirus, adeno-associated virus, vesicular stomatitis virus, herpes simplex virus, parvovirus, reovirus, vaccinia virus, Newcastle disease virus, measles virus and their variants. Regulatory RNAs with tumoricidal function useful in this invention include but are not limited to RNA motors, siRNA or shRNA silencers or RNA enhancers, microRNAs such as tumor suppressors miR34 and other therapeutic nucleic acids. Examples of suitable transgenes for use in the instant invention are included but not limited to those provided in Table 1. All of these transgenes and nucleotides may be operatively linked to cell penetrating peptides to promote transfer from the SS vehicle cells to the target tumor cells.

TABLE 1

| | Source/Reference |
|---|---|
| Tumoricidal Transgenes | |
| Granzymes A and B | Granzyme A: NCBI Reference Sequence: NM_006144.3 |
| | Granzyme B: GenBank: A26437.1 |
| Perforin | GenBank: M31951.1 |
| Granulysin | GenBank: BC023576.2 |
| TNFα | NCBI Reference Sequence: NM_000594.3 |
| IFNα | GenBank: M11003.1 |
| IFNγ | GenBank: U10360.1 |
| IL-12 | GenBank: AF101062.1 |
| Chemokines | |
| Superantigens and homologues | U.S. Pat. No. 6,126,945; U.S. Pat. No. 8,431,117 B2 |
| | Jarraud S et al., J. Immunol 166: 669-677 (2001) |
| Superantigen-tumor targeting fusion proteins | U.S. Pat. No. 7,125,554 |
| Pseudomonas A toxin-tumor targeting fusion | US 20120263674 A1; WO 2013040141 A1; U.S. Pat. No. 6,064,644; |
| proteins | EP0610286 B1; EP0261671 B1 |
| Dipetheria toxin-tumor targeting fusion proteins | Kreitman RJ. BioDrugs. 23: 1-13 (2009); |
| Shiga toxin and subtype B | |
| FADD1-90 Death Effector Domain (DED), | |
| Apaf-11-97 caspase recruitment domain (CARD), | |
| Apaf-11-97 (mApaf) CARD | |
| TRAIL | Srivastava et al., Neoplasia 3: 535-46 (2001) |
| Chimeric protein: dsRNA specific protein fused to a | U.S. Pat. No. 7,566,694B2 |
| caspase specific protein | |
| Complement membrane attack complex | |
| Oncolytic Viruses/Genomes | |
| Measles | Schneider et al., J Virol 74: 9928-36 (2000); |
| Reovirus | Roner et al., Proc Natl Acad Sci 98: 8036-8041 (2001) |
| Sindbis virus | Strauss et al,, J Virol 133: 92-110 (1984) |
| Semliki forest virus | Kaariainen et al., J. Cell Sci Suppl. 7: 231-250 30 (1987)); |
| Venezuelan equine encephalitis virus | Kinney et al, Virology 191: 569-580 (1992) |
| poliovirus | Racaniello et al., Proc. Natl. Acad Sci. 78: 4887-4891 (1981) |
| herpes simplex virus | NCBI GenBank (Z86099). |
| Newcastle disease virus | NCBI GenBank (AF309418) |
| Viruses or viral vectors encoding siRNAs or shRNAs | Tong SG Mol Ther 13: S118 (2006) |
| Viruses or viral vectors encoding precursor shRNAs | Xia HP J Cancer Mol 4: 79-89 (2008.) |
| of miRNAs | Schoof et al., Am J Cancer Res 2: 414-433 (2012) |
| Regulatory RNAs | |
| Nucleiic acids encoding siRNA | Deng Y et al., Gene. 538: 217-227 (2014) |
| Nucleiic acids encoding shRNA | Fellman C et al., Nat Cell Biol. 16: 10-8 (2014) |
| Nucleic acids encoding miRNA | Varble A, et al., Proc. Natl Acad. Sci. 107: 11519-11524 (2010) |
| RNA enhancers | Janowski BA et al., Nature Chem Biol 3: 166-173(2007) |
| microRNAs | Xi JJ Cancer Treat Res.; 158: 119-37 (2013) |
| Transposons | Zoltan I et al., Mobile DNA, 1: 1-15 (2010) |
| Viruses incorporating miRNAs | Grundhoff A et al., Virology 411 325-343 (2011) |
| | Varble A, et al., Proc. Natl Acad. Sci. 107: 11519-11524 (2010) |
| | Shapiro J et. al., RNA 16: 2068-2074 (2010), |
| Phi29 viral RNA motors | Guo et al., Human Gene Therapy 16: 10971109 (2005) |
| shRNAs targeting heme oxygenase | |
| shRNAs targeting nitrous oxide synthase | |
| shRNA targeting HIF1-α | |
| shRNAs targeting Bax, and Bcl-2 | Shen et al., Adv Cancer Res. 82: 55-84 (2001) |
| miRNAs targeting defective oncogenes, anti- | Xia HP J Cancer Mol 4: 79-89 (2008.) |
| apoptotic genes, pro-angiogenesis genes, pro- | Schoof et al., Am J Cancer Res 2: 414-433 (2012) |
| growth/prolifereation genes, pro-metastases genes | |

Oncolytic viral genomes that are useful in the above method include but are not limited to measles virus (Schneider et al, J Virol. 74:9928-36 (2000)); reovirus (Roner et al, Proc Natl Acad Sci 98: 8036-8041 (2001)); Sindbis virus (Strauss et al, J Virol 133:92-110 (1984)); Semliki forest virus (Kaariainen et al, J. Cell Sci Suppl. 7: 231-250 (1987)); Venezuelan equine encephalitis virus, (Kinney et ah, Virology 191:569-580 (1992)); Newcastle disease virus (NCBI GenBank AF309418); poliovirus; (Racaniello et al., Proc. Natl. Acad. Sci. 78: 4887-4891 (1981)); herpes simplex virus (NCBI GenBankZ86099). Viral vectors for this purpose may be viral genomic DNA or RNA or a fragment of a homologue of viral DNA or RNA.

The present invention contemplates that siRNA or shRNA silencers or RNA enhancers and microRNAs are packaged into virus/oncolytic viruses or viral genomes which are then inserted into SSEPCs directly or incorporated into the β-globin lentiviral vector for insertion into SSEPCs. In addition, several human viruses naturally exhibit or are engineered to functionally express microRNAs, siRNA or shRNA silencers or RNA enhancers or their precursor nucleotides. Such viruses have evolved miRNA repertoires to emulate host miRNAs enabling them to access and regulate host transcripts for cell growth and differentiation. These virus families mostly comprise DNA genomes, but at least some with RNA genomes, encode miRNAs. Such viruses include but are not limited to herpesviruses, numerous members of the polyomavirus family, as well as adenoviruses, ascovirus, and baculovirus (Grundhoff A et al., Virology 411 325-343 (2011). miRNAs useful in this invention are also integrated coding regions of natural viruses. A microRNA-124 hairpin inserted into an intron of the nuclear export protein transcript of influenza virus and showed robust expression and function without segment instability. The same locus was grafted into a duplicated non-essential subgenomic area of Sindbis virus resulting in non-canonical cytoplasmic-based processing independent of nuclear events (Varble A, et al., Proc. Natl Acad. Sci. USA 107, 11519-11524 (2010); Varble A, et al., RNA Biol. 8:190-4 (2011)). When transfected into SSEPCs or SSiPPSCs, the virus containing its miRNA cargo is packaged into vesicles and trafficks via endosomal-exosomal pathways to the viral synapse or viral receptors expressed on target tumor cells. From there the miRNA virus is endocytosed into the target tumor cell. Such transfer is facilitated by the adhesive interface between ICAM in the SSEPC and αᵥβ₃ overexpressed on target tumor endothelial cells (Pegtel M et al., Biochim Biophys Acta 809: 715-721 (2011). When miRNA is introduced into SSEPCs or SSiPPSCs via the β-globin vector, miRNAs are similarly packaged, vesiculated and secreted. In all cases, the machinery for viral and/or mRNA production and transfer is retained in mature SSRBCs emerging from SSEPC or iPPSCs. Such viruses incorporating functional miRNAs or their precursor nucleotides can be used alone, packaged into the an oncolytic virus or viral genome or the β-globin lentiviral vector for transduction of SSEPCs or SSiPPScs.

Target genes for the siRNAs, shRNA and microRNAs include any gene that promotes the multiplication and spread of a cancer cell. Such targets genes include but are not limited to the following examples: heme oxgenase, nitrous oxide synthase, p53, RAS, CXCR4, p-Catenin, bcl-2, PLK-1, Somatostatin, Raf-1, c-raf, EGFR, HER-2, VEGL, HIF1-1α, Skp-2. MMP-9+, Cathepsin, PLK1, VEGF-R2, EWS-FLI1, Rad51, c-myc MDM2, VEGF, FGF-4, EZH2, p110α. Applicable tumors are given in the section on applicable tumors for inventions disclosed herein and in Table 2 of US8, 431, 117 incorporated herein by reference in its entirety with its references.

SSEPCs or SSiPPScs or mature SSRBCs are used to treat murine and human tumors as disclosed in Animal Tumor Models and Examples 3, 4 and 5.

SSEPCs useful in this invention encompass all SS erythroid progenitor cells or other stem stems which are able to differentiate into mature SS cells. The blood lineage marker for erythroid progenitors is CD71. The human and mice hematopoietic cell markers for the commonly accepted type of hematopoietic stem cells are:

Mouse HSC: CD34^{lo/-}, SCA-1⁺, Thy1.1^{+/lo}, CD38⁺, C-kit⁺, lin⁻

Human HSC: CD34⁺, CD59⁺, Thy1/CD90⁺, CD38^{lo/-}, C-kit/CD117⁺, lin⁻

The signature SLAM code for the hemopoietic hierarchy are: megakaryocyte-erythroid progenitor (MEP): lin⁻SCA-1⁻c-kit⁺CD34⁻CD16/32^{low}. The cells in the erythroid series are derived from myeloid progenitor cells or from the bi-potential megakaryocyte-erythroid progenitor cells which eventually give rise to mature red blood cells. The erythroid progenitor cells develop in two phases: erythroid burst-forming units (BFU-E) followed by erythroid colony-forming units (CFU-E); BFU-E differentiate into CFU-E on stimulation by erythropoietin and then further differentiate into erythroblasts when stimulated by other factors.

In the process of red blood cell maturation, a cell undergoes a series of differentiations. The following stages of development all occur within the bone marrow. These stages correspond to specific appearances of the cell when stained with Wright's stain and examined by light microscopy, and correspond to other biochemical changes.
1. A hemocytoblast, a multipotent hematopoietic stem cell becomes
2. a common myeloid progenitor or a multipotent stem cell and then becomes
3. a unipotent stem cell, and then becomes
4. a pronormoblast, also commonly called an proerythroblast or a rubriblast and the becomes
5. a basophilic or early normoblast, also called an erythroblast and then becomes
6. a polychromatophilic or intermediate normoblast and then becomes
7. an orthochromatic or late normoblast. At this stage the nucleus is expelled before the cell becomes
8. a reticulocyte.

After Stage 7, the cell is released from the bone marrow. Thus, in newly circulating red blood cells there are about 1% reticulocytes. After one to two days, these ultimately become “erythrocytes” or mature red blood cells. In the process of maturation, a basophilic pronormoblast is converted from a cell with a large nucleus and a volume of 900 fL to an enucleated disc with a volume of 95 fL. By the reticulocyte stage, the cell has extruded its nucleus, but is still capable of producing hemoglobin.

To date, SSEPCs have not been conceived as useful in anti-tumor therapy. These nucleated SS erythroid progenitors contain SS hemoglobin which mimics the physiologic behavior of the mature SSRBCs in polymerizing under deoxygenating conditions of the tumor microenvironment. Like mature SSRBCs, the SSEPCs also home to hypoxic tumor microvessels, form microaggregates and obstruct tumor blood vessels. Tumor selectivity of the SSEPCs is conferred by their constitutive expression of adhesion receptor ICAM-4 whose cognate ligand αᵥβ3 is highly overexpressed on tumor endothelial cells. When trapped on these tumor endothelial cells SSEPCs also autohemolyze releasing toxic heme-related oxidants capable of inducing apoptosis in surrounding tumor tissue.

These properties distinguish SSEPCs from T cells and cytokine activated T cells (CITCs) used by themselves or as vehicles for carriage of tumoricidal agents into tumors. Such T cells and CITCs rely on genetically engineered insertion of single chain antibodies to confer tumor specificity. Whereas SSEPCs are known to circulate freely in sickle cell patients, less than 10% of administered T cells localize in tumors and largely deposit in spleen, liver and lung where no tumor antigen is expressed Importantly, unlike T cells or CITCz, SSEPCs do not produce toxicity-inducing cytokines or graft versus host disease which impede the successful use of T cells in adoptive tumor therapy and bone marrow transplantation. Thus, large numbers of SSEPCs can localize in tumor and deliver their TTORs into the tumor microenvironment without undue short or long range toxicity.

The SSEPCs are obtained from embryonic stem cells, induced pluripotent stem cells, bone marrow or peripheral blood of subjects with sickle cell anemia or its variants as described above. Preferably, the SS progenitors are transduced with the lentiviral β-globin vector incorporating TTORs or siRNAs, shRNAs or microRNAs are integrated into the β-globin coding region. This vector can accommodate bicistronic compositions. SSEPCs or SSiPPSCs can also be transduced directly with nucleic acids encoding TTORs using vectors other than those with the B-globin coding region. Both transduction methods induce the expression of TTORs in the SSEPCs and such SSEPCs are able to home to hypoxic tumors, adhere to tumor endothelium, form microaggregates under deoxygenating conditions and autohemolyze in a fashion similar to the mature SSRBC. In contrast, only mature SSRBCs derived from SSEPCs transduced with the lentiviral β-globin vector with packaged TTORS or silencing RNAs transfected into SSEPCs produce TTOR proteins or silencing RNAs along with SS hemoglobin. Moreover, the SSEPC and iPPSCs exhibit a robust vesicular transport system for transport of such tumoricidal TTOR molecules to target tumor cells. Trafficking of intracellular TTOR proteins or nucleotides to such mobile vesicles is promoted by agents fused to TTORS such as CD63 sorting signal SIRSGYEVM (SEQ ID NO:11) or the ligand for the mannose 6-PO₄ receptor on endosomes. In the SSEPC, formation of an adhesive junction with the tumor cells between ICAM-4 expressed on the SSEPC and αᵥβ3 overexpressed on tumor endothelial cells initiates streaming of golgi, vesicle polarization and microtubular vesicular transport to the synaptosome. Synapse formation also lowers the activation threshold enabling the vesicular transport system to transfer of TTORS stored in such vesicles to the junctional interface with tumor endothelial cells. Such vesicular storage of tumoricidal proteins protects the cell from lysis by cytotoxic transgene products and oncolytic viruses. Additional sequences fused to the tumoricidal cargo such as the translocation domain of pseudomonas exotoxin A (ETA) from pseudomonas aeruginosa comprising amino acids 253 to 366 and the transcytosis domain KKKVTAQELD (SEQ ID NO:12) of most staphylococcal enterotoxins and streptococcal pyrogenic toxin superantigens promote endosomal escape and translocation of the tumoricidal cargo to the synapse.

Within the closed compartment of this synaptic junction, TTOR-containing vesicles are exocytosed by the SSEPC or SSiPPSC into target tumor cells endosomes. Such a closed system enables transport of biological agents to target tumor cells without interference by seroreactive neutralizing antibodies (NAs) or inhibitors. In the target tumor cells, the tumoricidal cargo is released from the endosomes into the cytoplasm where it is free to induce apoptosis. The presence of perforin in the target enhances the transport and egress of oncolytic viruses to the cytoplasm by punching holes in the endosomal vesicle where the virus is stored. Additional agents such as adenoviruses, choloquine and ammonium chloride are useful to enable the release of tumoricidal proteins from the secretory granules into the cytoplasm from which they can induce apoptosis via caspase and non-caspase dependent mechanisms.

For oncolytic viruses that infect SSEPCs or iPPSCs the cellular machinery for cell to cell communication of such viruses pre-exists, but is not activated. When activated by a combination of viral and adhesive interactions with target tumor endothelium or tumor cells, the viral infected SSEPC hijacks elements of the host cell to polarize microtubular vesicular trafficking pathways toward the synapse. Such trafficking facilitates viral exocytosis and dissemination between cells. In this process, the virological synapse (VS), defined as a cytoskeleton-dependent, stable adhesive junction across which virus is transmitted by directed transfer plays a critical role. This VS forms in response to contact between virally infected (effector) and uninfected (target) tumor cells or tumor endothelial cells and contains viral antigens and cellular adhesive receptors colocalized at the synaptic interface. The tumor endothelial cell contributes to this process by recruiting and expressing viral and adhesion receptors at the synaptic surface.

In SSEPCs and iPPSCs transduced with oncolytic virus, the virus replicates and trafficks to the tumor site where it adheres to the tumor endothelium. At this point, the lyticr release phase of the viral life cycle is activated resulting in spillage of an abundant supply of virus for infection of tumor endothelial cells or tumor cells. Both RNA and DNA oncolytic viruses housed within the SSEPC or iPPSC can bud from their cell membranes and fuse with the target cell membrane or they may be transferred by virologic synapse as described above. Alternatively, such viruses can be released from the cell membrane in exosome like structures that can be endocytosed by the target cells.

The TTORs are integrated into SSEPCs iPPSCs using methods described above. In addition, human and murine SS erythroid progenitor cells and human pluripotent stem cells from sickle cell donors are transduced with TTORS directly or with β-globin lentiviral vector comprising TTORs as described below. The transduced SSEPCs are used in therapy of cancer in animal tumor models as described in the section on Animal Tumor Models and in humans as described in Examples 2, 3 and 5.

Isolation, Purification and Enrichment of the Murine SCA+ Bone Marrow Cells from Sickle Cell Mice

Bone marrow cells from 8- to 10-week-old humanized homozygous sickle mice are harvested from femurs and tibias. Single cells resuspended in 5 ml syringe with 21 gauge needle and cell debris and bone fragments removed by filter through 40 μm cell strainer. Scar cells are selected by immunomagnetic selection using the EasySep kit (Stem Cell Technologies). Procedure is used for processing single bone marrow cell suspension at a concentration of 2.5×10⁸ cells/mL in PBS with 0.5% FBS and 2 mM EDTA medium. Sca1 PE labeling reagent is added at 50 μL/mL, mixed well and incubated at room temperature for 15 minutes. PE Selection Cocktail is added at 70 μL/mL at room temperature for 15 minutes, and final by add the nanoparticles at 50 μL/mL for 10 minutes before column purification. The LS column (Milenyi Biotec Inc.) is rinsed with 3 ml of the same medium before apply the cells to the column LS column is washed with 3×3 ml of medium to remove unlabeled cells. Five ml medium is added to the LS column on a new collection tube. the Sca1+ cells are collected and counted in a hemocytometer.

### Isolation, Purification Expansion and Enrichment of Murine Sca1⁺ SSEPCs

Bone marrow cells from 8- to 10-week-old humanized homozygous SS mice are harvested from femurs and tibias. Cells are resuspended in 5 ml syringe with 21 gauge needle, and cell debris and bone fragments removed by filter through 40 μm cell strainer. Scar cells are selected by immunomagnetic selection using the EasySep kit (Stem Cell Technologies). Single bone marrow cell suspension contains 2.5×10⁸ cells/mL in PBS with 0.5% FBS and 2 mM EDTA medium. The cells are applied to an LS column (Milenyi Biotec Inc.), collected and counted cell on a hemocytometer. These cells are transduced with tumoricidal transgenes or oncolytic virus or RNA nucleotide regulators as described above and used for reconstitution of irradiated SS mice. After a rest period of 6-14 weeks to allow full reconstitution, tumors are implanted and tested as described below.

Testing SSEPCs and Mature SSRBCs Transduced with Granzyme-Perforin-β2-Ad Vector in the Murine 9646 Neuroblastoma Model

C57BL/6 female mice 6-7 weeks old obtained from Jackson Laboratories will be maintained in animal facilities under UAACCD regulations. The cultured 9646 neuroblastoma cells will be tested for tyrosine hydroxylase expression and secretion of catacholamines by well established methods (34). The SSRBCs transduced with granzyme-perforin-β2-Ad construct will also be tested for their ability to secrete granzyme and perform in response to graded doses of catacholamines in vitro according to a previously described model (23,24). Similarly, we will also test the ability of these cells to adhere to αᵥβ₃ after activation by epinephrine (23-24). For in vivo experiments, tumor cells will be harvested during exponential growth, washed and resuspended in media. Cell viability will be greater than 90%. Mice will be irradiated with 1200 Gy and immediately reconstituted with 10⁶ SCA+ enriched erythroid progenitor cells from SS mice that have been transduced with β-globin lentiviral expression vector in which the β-globin coding region has been replaced by nucleic acids encoding granzyme, perforin and the β2-AR under control of the HIF1α promoter. The mice will be rested for 8-14 weeks. At a point when peripheral blood samples show expression of granzyme, perforin and β2-adrenergic receptor in up to 60% of SSRBCs the mice will be implanted with neuroblastoma (10⁵ in 100 μl of media) subcutaneously in the right flank. Tumors will be measured 3×/week using the formula: length×(width)²/2. Endpoint is tumor volume of 1500 mm³. Toxicity shall be assessed by daily weights and behavioral changes as described above. All tumors, tumor implantation sites and organs will be collected at the end of the study for histopathology and analysis of tumor cell apoptosis by the annexin assay.

Generation of SSEPCs and Mature SSRBCs from SSiPPSCs and Transfection with TTORs

TTORS can be introduced into Pluripotent Stem Cells (iPPSC) from sickle cell disease patients using the lentiviral β-globin vector described above. These SSiPPSCs can then be differentiated into SSEPCS or mature SSRBCs. Two procedures for generation of SSiPPSCs are provided below. Additional procedures for generation of SSiPPSCs from patients with homozygous sickle cell cell anemia are provided in US patent application 20130017596 filed May 25, 2012 and US patent application 20100150889 filed Dec. 17, 2009 which are incorporated by reference with their references in entirety. Such SSiPPSCs can also be derived from patients with AA hemoglobin that are transformed by gene editing using the CRISPR-caw or TALE or Zinc finger nuclease methodology described herein.

### Generation and Characterization of SSiPPSCs (Additional Protocols for Generation of SSiPPCs

Protocol 1.

The normal human adult iPPSC (hiPSn1) cell line is generated from human adult fibroblasts (FD-136). Briefly, it was established from a healthy 25-year old woman who gave her informed consent to the procedure. Plasmids pSin-EF2-Oct4-Pur, pSin-EF2-Sox2-Pur, pSin-EF2-Nanog-Pur and pSin-EF2-Lin28-Pur13 are used for reprogramming. The human sickle cell disease iPPSC (hiPSSCD) cell line is generated from human amniotic fluid cells (hAFC) obtained from pregnant women undergoing diagnostic amniocentesis. Briefly, hAFC from sickle cell anemia fetuses are collected with the informed consent of the parents and used for reprogramming to hiPPSC. A homozygous HbS mutation in hAFC is confirmed by sequencing. hiPPSC are generated according to the technique of Yu et al. 2 using lentiviral supernatants for the reprogramming genes Lin28, Nanog, Sox2 and Oct4 employing pSIN-EF2 plasmids2 (Addgene). The hiPPSC line from sickle cell disease is called PB04 and is recorded in the European registry (www.hesreg.eu). An additional protocol for generation of SSiPPScs is provided below

Protocol 2.

Human fetal lung fibroblasts IMR-90 are retrieved from the ATCC (Manassas, Va., USA) and adult hiPPSC are generated using a skin primary fibroblast cell line established from a healthy 25-year old woman after informed consent (FD136 kindly provided by A. Munnich, Inserm U781, Paris, France) and plasmids pSin-EF2-Oct4-Pur, pSin-EF2-Sox2-Pur, pSin-EF2-Nanog-Pur and pSin-EF2-Lin28-Pur13 from Adgene (Cambridge, USA). Virus production is performed by Vectalys (Labège, France). hiPPSC clones are obtained as follows. Briefly, 200,000 fibroblasts are infected 1 day after plating with the four lentiviral vectors at the highest possible MOI between 7 and 23 depending on the original virus preparation, in the presence of polybrene at 8 mg/mL (Sigma). Two days later, viruses are removed and medium progressively changed to hESC medium in the following week. The medium is then changed on a daily basis, as for hESC. hiPPSC colonies appear between 3 and 6 weeks after infection and are selected and clonally amplified. hiPPSC clones are characterized using different techniques: karyotypes are determined by multi-fluorescence in situ hybridization and gene expression by either flow cytometry or by real-time polymerase chain reaction (PCR) and Taqman low density arrays. Briefly, RNA is extracted from cells using an RNeasy kit (Qiagen); 1 mg of total RNA is retrotranscribed using SuperScript (Invitrogen) enzyme and the expression of markers is analyzed using the comparative DDCt method with GAPDH as the endogenous control and a human embryonic carcinoma 2102EP (hEC) or a normal hESC sample as a calibrator (HUES-24 line; kindly provided by M. Pucéat, INSERM UMR861, Evry, France). Endogenous and exogenous expression of OCT4, SOX2, LIN28 and NANOG is evaluated in hiPPSC clones. In vitro differentiation of hiPPSC clones was appreciated by the formation of hEB. Briefly, hiPPSC are recovered from dishes using collagenase (1 mg/mL; Invitrogen) and transferred to low attachment plates (Nunc, Dutscher, Brumath, France) in the same medium without basic fibroblast growth factor. Media are changed every 2-3 days. After 10 days, hEB are retrieved and lysed for RNA extraction and quantitative real-time PCR (qRT-PCR). Markers for each embryonic layer are analyzed (ectoderm: Pax6, ck18; mesoderm: brachyury, Gata4, RunX1, CD34, Nkx2.5, KDR; endoderm: ck17, AFP).

### Cells and Culture Conditions

hiPPSC (IMR90)-16 (n=3) passages 13-21, hiPPSC (FD-136)-25 (n=2) passages 25-32 and hESC H1 (n=3) (National Institute of Health code WA-01) passages 23-48 are grown on primary mouse embryonic fibroblasts treated with mitomycin (20 mg/mL).

Generation of Human SSEPCs and Mature SSRBCs from SS Embryonic Stem Cells (hSSESCs) and Transfection with TTORs

SSEPCs and mature SSRBCs are obtained from SSESCs using the following techniques. Four human SSESC lines are established from umbilical cord blood of patients with sickle cell anemia. Four human ESC lines from normal donors are also useful as follows: H1 (National Institutes of Health-registered as WA01), MA01 and MA99 (derived at Advanced Cell Technology), and HuES-3 (obtained from the Harvard Stem Cell Institute). hESCs are grown on mitomycin C-treated mouse embryonic fibroblast (MEF) in complete hESC media until they reach 80% confluence. A 4-step procedure is used for the generation and expansion of SS erythroid cells (EBCs) from hSSESCs.

### Step 1. Erythroblast (EB) Formation and Hemangioblast Precursor Induction (Day −3.5 to 0.

To induce hemangioblast precursor (mesoderm) formation, EBs are formed by plating 1 well of hESCs per EB culture well (ultra-low 6-well plates; Corning, Corning, N.Y.) in 3 to 4 mL serum-free

Stemline media (Sigma-Aldrich, St Louis, Mo.) with BMP-4, VEGF165 (50 ng/mL each; R&D Systems, Minneapolis, Minn.), and basic fibroblast growth factor (bFGF, 20 ng/mL; Invitrogen, Carlsbad, Calif.). Half of the media is refreshed 48 hours later with the addition of stem cell factor (SCF), thrombopoietin, and FLT3 ligand (20 ng/mL each; R&D Systems).

### Step 2. Erythroblast (EB) or Hemangioblast (HB) Expansion (Days 0-10)

After 3.5 days, EBs are collected and dissociated with trypsin. A single cell suspension is obtained by passing the cells through a G21 needle 3 times and filtering through a 40-μm filter. After resuspending in Stemline II medium, the cells are mixed with blast-colony growth media (BGM; 5×10⁵ cells/mL) and plated in 100-mm ultra low dishes (10 mL/dish). The cultures are expanded for 9 to 10 days in BGM. The addition of 20 ng/mL bFGF and 2 μg/mL recombinant tPTD-HoxB4 fusion protein to BGM significantly enhances hematopoietic cell proliferation. HoxB4 protein

promotes hematopoietic development in both mouse and human ESC differentiation systems. The grape-like blast colonies are usually visible by microscopy after 4 to 6 days and expanded rapidly outward. Additional BGM is added to keep the density of blast cells at 1 to 2×10⁶ cells/mL.

### Step 3. Erythroid Cell (ES) Differentiation and Expansion (Days 11-20)

At the end of step 2, the cell density is often very high (≧2×10⁶/mL). Equal volumes of BGM, containing 3 units/mL erythropoietin (Epo; total Epo is 6 units/mL) without HoxB4, are added to supplement the existing BGM. The blast cells are further expanded and differentiated into erythroid cells (ESs) for an additional 5 days. For further expansion, the erythroid cells are transferred into 150-mm Petri dishes and Stemline II-based medium containing SCF (100 ng/mL), Epo (3 unit/mL), and 0.5% methylcellulose added every 2 to 3 days. (When the cells reached confluence, they are split at a ratio of 1:3 to allow maximum expansion for an additional 7 days [cell density, 2-4×10⁶/mL].)

### Step 4. Enrichment of Erythroid Cells (Day 21)

Erythroid cells obtained from step 3 are diluted in 5 volumes of Iscove modified Dulbecco medium (IMDM) plus 0.5% bovine serum albumin (BSA) medium and collected by centrifugation at 1000 g for 5 minutes. The cell pellets are washed twice with IMDM containing 0.5% BSA and plated in

tissue culture flasks overnight to allow nonerythroid cells (usually the larger cells) to attach. The nonadherent cells are then collected by brief centrifugation. Plating in BGM after the 3.5-day EB dissociation step is denoted as day 0 of erythroid culture. The time period for the entire procedure is 19 to 21 days from the plating of EBs in BGM, with a final culture volume of 3 to 4 L for 5 to 6×10⁶ MA01 hESCs. The efficiency of RBC generation from MA99, H1, and HuES-3 is approximately 5 to 6 times less than from MA01 hESCs (with a correspondingly lower final culture volume). SSEPCs obtained from this procedure (before put into culture for further maturation and enucleation) are used for functional characterization, flow cytometry, and hemoglobin analyses. The large-scale culture experiments are carried out with hESC lines MA01 (n=6), H1 (n=2), HuES-3 (n=2), and MA99 (n=1). For further maturation, cells collected at days 18 to 19 (step 3) are diluted with IMDM containing 0.5% BSA (1:5 dilution) and centrifuged at 450 g for 10 minutes. To partially enrich the cells for RBCs, the top white portion of cell pellet is removed using a pipette with a long fine tip. The RBCs were then plated in StemPro-34 SCF (Invitrogen) medium containing SCF (100 ng/mL) and Epo (3 unit/mL) at a density of 2×10⁶ cells/mL. The cells are cultured 6 days with media changes every 2 days and then switched to StemPro-34 containing Epo (3 unit/mL) for 4 to 5 more days. These cells are used for β-globin chain and benzidine stain analyses.
Erythroid Induction and Differentiation of ESCs and iPPSCs

The RBC generation protocol is adapted from one used to obtain ihESC and hiPPSC derived from normal fetal and adult fibroblasts. It comprises two steps: 1) differentiation of hiPPSC by formation of embryoid bodies (hEB) during 27 days in a liquid culture medium (LCM) on the basis of IMDM (Biochrom), 450 μg/m holo-human transferrin (Scipac), 10 mg/mL recombinant human insulin (Incelligent SG; CellGen), 2 IU/mL heparin, and 5% human plasma in the presence of stem cell factor (SCF; 100 ng/mL), thrombopoietin (TPO; 100 ng/mL), FLT3 ligand (FL; 100 ng/mL), rhu bone morphogenetic protein 4 (BMP4; 10 ng/mL), rhu vascular endothelial growth factor (VEGF-A165; 5 ng/mL), interleukin-3 (IL-3; 5 ng/mL, interleukin-6 (IL-6; 5 ng/mL) (Peprotech)

and erythropoietin (Epo; 3 U/mL) (Eprex, kindly provided by Janssen-Cilag, France); 5 and 2) differentiation/maturation of Day 27 hEB into RBC in the presence of the following sequential combination of cytokines: i) Day 0 to Day 8, dissociated EB are plated at a density of 0.1-1×10⁶ cells/mL in LCM containing 5% human plasma, 2 IU/mL heparin, SCF (100 ng/mL), IL3 (5 ng/mL) and Epo (3 IU/mL); ii) Day 8 to Day 18, the cells are resuspended at 0.3-1×10⁶ cells/mL and cultured in fresh medium supplemented with 10% human plasma, 4 IU/mL heparin, SCF, IL3 and Epo; iii)
Day 18 to Day 25, the cells are resuspended at 2-4×10⁶ cells/mL and cultured in fresh medium containing Epo. The cultures are maintained at 37° C. in 5% CO₂ in air. Cells are spun onto a glass slide by cytocentrifugation and stained with May-Grünwald-Giemsa (MGG) and new methylene blue reagents (Sigma) for morphological analyses. A Day-25 enucleated population purified by passage through a deleukocytation filter (Leucolab LCG2, Macopharma, Tourcoing, France) is used to determine the functionality of RBC. Our protocol comprises two steps: (i) differentiation of hiPPSC
by formation of human embryoid bodies (hEB) and (ii) differentiation/maturation to the stage of mature cultured RBC in the presence of cytokines.
Mature SSRBCs Derived from SShiPPSCs or SShESCs and Transduced with TTORs are Universal Donors

Preferably, SSiPSCs and SSESCs transduced with TTORs are derived from patients with blood type O, RH− blood (O−) which are also Kell, C, D and E negative. Such mature SS cells derived therefrom are compatible with all human patients and can be used as a universal donor for patients with solid tumors. SSEPCs require HLA typing for compatibility with recipients. In HLA histocompatibility testing matching for the major HLA subgroups a, b and c subgroups is sufficient for compatibility in up to 80% of the population (Nakatsuji N et al., Nature Biotech 26: 739-740 (2008)).

### Construction of the β-Globin Lentiviral Expression Vector and Introduction of Tumoricidal Transgenes

A 2.3-kb recombinant human β^{AS3} gene and 3.4 kb of human β-globin locus control region (LCR) sequences are subcloned into pWPT-GFP (a kind gift from Dr Didier Trono) replacing the EF1α promoter and green fluorescent protein (GFP). This self-inactivating (SIN) vector contains a deletion in the U3 region of the 3′ long terminal repeat (LTR) from nucleotide 418 to nucleotide 18 that inhibits all transcription from the LTR. The β^{AS3}-globin gene contains 266 bp of promoter, the 260-bp PstI 3′ globin enhancer, and a 375-bp RsaI fragment deletion of IVS2. DNase I hypersensitive site (HS) fragments, 5′ HS4, 3, and 2 were amplified by polymerase chain reaction (PCR) from a 22-kb fragment of the LCR. Nucleotide coordinates from GenBank accession no. U01317 are: HS4 592-1545, HS3 3939-5151, and HS2 8013-9215. The entire HS4, 3, 2β-globin gene construct is verified by sequencing. The exon 1 and 2 of human β^{AS3} gene are removed by PCR deletion, and replaced with restriction sites, XhoI and BsiwI, for new gene insertion.

### Preparation of the Granzyme/Perforin-β2-Ad-HIF1-α Vector and SEG/SEI-HIF1α Lentiviral Vector for Transduction of Murine SSEPCs and Human SSEPCs and SSiPPSs

Construction of the polycistron using PTV1 2A sequences and fusion PCR was performed essentially as described (Hoist J, et al., Nat Protoc 1:406-417 (2006)). Briefly, mouse Adrb2 cDNA (Clone 1349283; Thermo Scientific) was PCR amplified and modified with primers Adrb2-F and Adrb2-R to contain a 50 bp homology to human HIF1α 5′ UTR and a Kozak consensus sequence. At 3′ end the Adrb2 stop codon was eliminated and replaced with nucleotides (nt) from PTV 1 2A that will form a 22-nt overlap with the 50 bp end of the Gzmb amplicon. Mouse GzmbcDNA (Clone 3592898; Thermo Scientific) was PCR amplified and modified with primers Gzmb-F (the first 20 amino acids from ATG was removed) and Gzmb-R to overlap with the 30 end of the Adrb2 amplicon and to append 2A nt sequences upstream of the Gzmb. At the 30 bp end, the Gzmb stop codon was eliminated and replaced with nt from PTV1 2A that will form a 22-nt overlap with the 50 end of the Prf1 amplicon. Mouse Prf1 cDNA (Clone 40130577; Thermo Scientific) was PCR amplified and modified with primers Prf1-F and Prf1-R to overlap with the 30 end of the Gzmb amplicon and to append 2A nt sequences upstream of the Prf1 ATG. At the 30 end, the Prf1 stop codon was retained and Swa I restriction sites were added. After PCR the individual amplicons were gel purified and used in a three-element fusion PCR at a 1:100:1 (Adrb1/Gzmb/Prf1) molar ratio along with primers Adrb2-F and Prf1-R to produce a 3,764-bp amplicon containing the polycistron. The polycistron was gel purified and cloned into the general cloning vector pBS-SK+ (Stratagene) using the SmaI restriction sites (enzymes from New England Biolabs) to produce pJS-AGP and sequenced for authenticity. The human HIF-1α promoter and 5′ UTR was amplify by PCR with 50 bp overlap to 5′ of Adrb2-Gzmb-Prf1 polycistron gene, and assemble the HIF-1α-Adrb2-Gzmb-Prf1 by fusion PCR. Subsequently, the polycistron was subcloned into a SwaI site in the lentiviral vector to produce the HIF-1α-Adrb2-Gzmb-Prf1 polycistronic lentiviral vector which was sequenced for authenticity.

By the same strategy we produced a second polycistronic lentival vector HIF-1α-SEG-SEI. PCR reactions were performed using PrimeStar polymerase (Takara, Otsu, Japan, http://www.takara.co.jp). SEG and SEI coding sequences were connected with a short picornavirus 2A peptide sequence. All of the oligos used in this study were synthesized by Integrated DNA Technologies (Coralville, Iowa, http://www.idtdna.com/Home/Home.aspx) and all DNA gel extractions were performed using QIAquick Gel Extraction Kits (Qiagen, Hilden, Germany). Use of SS progenitor cells or mature SS erythrocytes engineered to express P2 adrenergic receptor linked to perforin and granzyme

The present invention contemplates that tumors with adrenergic receptors capable of responding to adrenergic stimuli and secreting adrenergic hormones such as epinephrine and norepinephrine including but not limited to neuroblastomas and pheochromocytomas are particularly sensitive to SS cells, SS progenitors and SS variants loaded or not with the tumoricidal agents or drugs disclosed herein and in preceding U.S. patent application Ser. Nos. 12/586,532, 12/276,941, 12/145,949. These tumors contain some of the synthetic and neurotransmitter properties present in more mature synaptic cells from sympathetic or parasympathetic ganglia. The presynaptic component is represented by cells which produce a wide variety of neuortransmitters including catacholamines exhibiting constitutive catecholamine secretion and adrenergic receptors (e.g., alpha and beta) for these hormones. Indeed, 90-95% of neuroblastomas show elevated serum levels of epinephrine, norepinephrine, dopamine and their metabolites homovanillic acid and vanilmandelic acid in the urine.

The present invention contemplates SS cells showing enhanced deposition in and killing of tumors including but not limited to neuroblastoma and pheochromocytoma which secrete catacholamines such as epinephrine and dopamine Catacholamines secreted from tumor cells activates β2 adrenergic receptor on SS RBCs upregulating BCAM/Lu and ICAM-4 expression via cAMP and protein kinase A (PKA)-mediated phosphorylation. BCAM/Lu- and ICAM-4 mediate SS RBC adhesion to laminin and αᵥβ₃ respectively in tumor vasculature. Thus SS cells are exposed to catacholamines secreted by these tumors as they circulate through these tumors and thereby primed to bind to the intrinsically upregulated tumor vasculature. For the effects of catacholamines and adrenergic hormones on SS cells (Hines P C et al., Blood. 101:3281-7 (2003); Zennadi R et al., Blood 104:3774-81 (2004) both of which are incorporated by reference with their references. The anti-tumor effect of the upregulated SS cells is enhanced by the use of a heme oxygenase inhibitor and chemotherapy as described above. Catecholamine-secreting tumor models used to test efficacy of the SS cells and HO-1 inhibitors include the SH-SY5Y neuroblastoma in SCID mice, C1300 neuroblastoma and numerous others neuroblastoma and pheochromocytoma models as described in ATCC catalogue of tumors (2009). Methodology for preparation and assessment of anti-tumor effects in these models is similar to that described for the tumor models disclosed herein under “Tumor Models.”

The present invention contemplate genetically engineered SS erythroblasts are transfected with a lentiviral vector as described above in which the β-globin coding region contains nucleic acids encoding the Beta-2 adrenergic receptor in frame with nucleic acids encoding perforin and granzyme b. The method for integrating these nucleic acids into the lente viral vector is described above. The SS erythroblast is preferentially SCA+. After transfection, the cells can be expanded and differentiated into mature SS cells. Alternatively, the SS erythroblasts can undergo expansion/differentiation in vivo after injection into lethally irradiated mice. Approximately 2 months after transplantation, the mature SS cells express the beta 2 adrenergic receptor along with granzyme and perforin. These cells are useful in the treatment of catacholamine secreting tumors such as neuroblastomas and pheochromocytomas. When injected into hosts bearing these tumors, the SS cells traverse the microcirculatory environment of these tumors which is enriched in catacholamines secreted by these tumor particularly under hypoxic condition. Catacholamines activate the P2 adrenergic receptor on the SS cells. This leads to the phosphorylation and expression of ICAM-4 on the SS cell surface and to the synthesis of perforin and granzyme b. ICAM-4's cognate adhesion receptor αᵥβ₃ integrin is expressed on tumor endothelial cells and VLA-4 is expressed on fibronectin abundant in the tumor vascular matrix. As the SSRBC or SSEPC adhesion receptor ICAM-4 synapses with its cognate ligand on endothelial cells and tumor matrix, granzyme b and perforin are released into the synaptic space and permeate the target tumor cells. The two enzymes thereupon work synergistically to induce apoptosis in the tumor endothelial cell. ICAM-4 further binds to Mac 1 and LFA-1 residues on polymorphonuclear cells which results in additional expansion of tumor microvascular aggregates and release of potent intracellular proteases and oxidants from PMNs.

The coding regions of the β2-adrenergic receptor, perforin and granzyme are linked with 2A peptides in the same order. The integration of these coding regions into the β-globin coding region of the lente viral vector is described above. Nucleic acids encoding pseudomonas exotoxin fragments/derivatives or native superantigens/homologues/fusion proteins or any other tumor cytotoxin e.g., diptheria, pertussis etc. may be linked to the β2-adrenergic receptor in place of perforin and granzyme b and also induce tumoricidal effects.

Therapeutically, the engineered SS cells are tested in the therapeutic models of established tumors under Animal Tumor Models. Animals are treated when the tumors reach a size of 0.5 cm in long diameter. The engineered SS cells are suspended in PBS containing Ca⁺⁺ and Mg⁺⁺ and administered intravenously in a volume of 200-300 μL every other day for a total of 3-5 treatments. Tumors are measured every other day. The endpoint is reached when the tumors reach 5 times their initial volume. In humans, The engineered SS cells are given as a conventional blood transfusion using SS cells that are ABO compatible with recipient erythrocytes. Alternatively, they may be given as a stem cell transplant using SCA+ hematopoietic cells transfused into irradiated donors. ABO blood typing for conventional erythrocyte transfusions and methodology for hematopoietic stem cell transfers in human are well established in the art. Transfusion of these bioengineered SS cells in mouse tumor models is described in section on Tumor Models along with outcomes. Clinical trial protocols and outcomes using these bioengineered SS cells as a transfusions or stem cell transplants are given in Examples 1, 2 and 3.

The wild type or engineered SS cells as described herein are also be useful for the treatment or prevention of metastases from the tumors described above either alone or transduced with tumoricidal transgenes or receptors or in combination with an HO-1 inhibitor and/or SEs as disclosed herein or chemotherapy/radiation as disclosed herein. The SS cells can be administered before, during, or after HO-1 inhibitors or SEs or chemotherapy/radiotherapy as disclosed herein. Treatment of a tumor with surgery, photodynamic therapy, radiation and/or chemotherapy is followed by administration of the SS cells compositions to extend the dormancy of micrometastases and to stabilize and inhibit the growth of any residual primary tumor or metastases. The compositions can be administered before, during, or after radiotherapy; before, during, or after chemotherapy; and/or before, during, or after photodynamic therapy.

ORIGIN

Mus musculus perforin 1 (pore forming protein), GenBank: BC137962.1
Proc. Natl. Acad. Sci. U.S.A. 99 (26), 16899-16903 (2002)
(SEQ ID NO: 8)

| | |
|---|---|
| 1 | cgtcttggtg ggacttcagc tttccagagt ttatgactac tgtgcctgca gcatcatggc |
| 61 | cacgtgcctg ttcctcctgg gccttttcct gctgctgcca cgacctgtcc ctgctccctg |
| 121 | ctacactgcc actcggtcag aatgcaagca gaagcacaag ttcgtgccag gtgtatggat |
| 181 | ggctggggaa ggcatggatg tgactaccct ccgccgctcc ggctccttcc cagtgaacac |
| 241 | acagaggttc ctgaggcctg accgcacctg caccctctgt aaaaactccc taatgagaga |
| 301 | cgccacacag cgcctacctg tggcaatcac ccactggcgg cctcacagct cacactgcca |
| 361 | gcgtaatgtg gccgcagcca aggtccactc cacggagggt gtggcccggg aggcagctgc |
| 421 | taatatcaat aacgactggc gtgtggggct ggatgtgaac cctaggccag aggcaaacat |
| 481 | gcgcgcctcc gtggctggct cccactccaa ggtagccaat tttgcagctg agaagaccta |
| 541 | tcaggaccag tacaacttta atagcgacac agtagagtgt cgcatgtaca gttttcgcct |
| 601 | ggtacaaaaa cctccactcc accttgactt caaaaaggcg ctcagagccc tcccccgcaa |
| 661 | ctttaacagc tccacagagc atgcttacca caggctcatc tcctcctatg gcacgcactt |
| 721 | tatcacggct gtggacctcg gtggccgcat ctcggtcctt acagccctgc gtacctgtca |
| 781 | gctgaccctg aatgggctca cagctgatga ggtaggagac tgcctgaacg tggaggccca |
| 841 | ggtcagcatc ggtgcccaag ccagcgtctc cagtgaatac aaagcttgtg aggagaagaa |
| 901 | gaaacagcac aaaatggcca cctctttcca ccagacctac cgtgagcgtc acgtcgaagt |
| 961 | acttggtggc cctctggact ccacgcatga tctgctcttc gggaaccaag ctacaccaga |
| 1021 | gcagttctca acctggacag cctcactgcc cagcaaccct ggtctggtgg actacagcct |
| 1081 | ggagcccctg cacacattac tggaagaaca gaacccgaag cgggaggctc tgagacaggc |
| 1141 | tatcagccat tatataatga gcagagcccg gtggcagaac tgtagcaggc cctgcaggtc |
| 1201 | aggccagcat aagagtagcc atgattcatg ccagtgtgag tgccaggatt caaaggtcac |
| 1261 | caaccaggac tgctgcccac gacagagggg cttggcccat ttggtggtaa gcaatttccg |
| 1321 | ggcagaacat ctgtggggag actacaccac agctactgat gcctacctaa aggtcttctt |
| 1381 | tggtggccag gagttcagga ccggtgtcgt gtggaacaat aacaatcccc ggtggactga |
| 1441 | caagatggac tttgagaatg tgctcctgtc cacaggggga cccctcaggg tgcaggtctg |
| 1501 | ggatgccgac tacggctggg atgatgacct tcttggttct tgtgacaggt ctccccactc |
| 1561 | tggtttccat gaggtgacat gtgagctaaa ccacggcagg gtgaaattct cctaccatgc |
| 1621 | caagtgtctg ccccatctca ctggagggac ctgcctggag tatgcccccc aggggcttct |
| 1681 | gggagatcct ccaggaaacc gcagtggggc tgtgtggtaa cataataaca acaataacat |
| 1741 | gcctgagagc tgggtgtagt agcacacgcc tttaatccca gcatttggga ggcagagaca |
| 1801 | ggtggatatc tatgagttcg aggccagcct gggtctacag ggtctcaaaa aaaaaaaaag |
| 1861 | caaacaacaa aactggaatg ttcaactggc ttctccctgg ggatctgcaa tggcttacta |
| 1921 | tgcatagaga ggccactaga gtggctgagt ttttacaata gagcatccct gactttccct |
| 1981 | tccacactgc ctcagccctg caatgcccga aagcttggca actactgcca cgaagcgtaa |
| 2041 | acatgggcca ggagcc |

Mus musculus granzyme B (Gzmb), mRNA. NCBI Reference Sequence:
NM_013542. J. Immunol. 188 (8), 3886-3892 (2012) (SEQ ID NO: 9)

| | |
|---|---|
| 1 | agagggggta caaggtcaca gagccccctc tgccttcttc ctctcctaga ggttaaaaga |
| 61 | gagcaaggac aacactcttg acgctgggac ctaggcggcc ttccggggaa gatgaagatc |
| 121 | ctcctgctac tgctgacctt gtctctggcc tccaggacaa aggcagggga gatcatcggg |
| 181 | ggacatgaag tcaagcccca ctctcgaccc tacatggcct tactttcgat caaggatcag |
| 241 | cagcctgagg cgatatgtgg gggcttcctt attcgagagg actttgtgct gactgctgct |
| 301 | cactgtgaag gaagtataat aaatgtcact ttgggggccc acaacatcaa agaacaggag |
| 361 | aagacccagc aagtcatccc tatggtaaaa tgcattcccc acccagacta taatcctaag |
| 421 | acattctcca atgacatcat gctgctaaag ctgaagagta aggccaagag gactagagct |
| 481 | gtgaggcccc tcaacctgcc caggcgcaat gtcaatgtga agccaggaga tgtgtgctat |
| 541 | gtggctggtt ggggaaggat ggccccaatg ggcaaatact caaacacgct acaagaggtt |
| 601 | gagctgacag tacagaagga tcgggagtgt gagtcctact ttaaaaatcg ttacaacaaa |
| 661 | accaatcaga tatgtgcggg ggacccaaag accaaacgtg cttcctttcg gggggattct |
| 721 | ggaggcccgc ttgtgtgtaa aaaagtggct gcaggcatag tttcctatgg atataaggat |
| 781 | ggttcacctc cacgtgcttt caccaaagtc tcgagtttct tatcctggat aaagaaaaca |
| 841 | atgaaaagca gctaactaca gaagcaacat ggatcctgct ctgattaccc atcgtcccta |
| 901 | gagctgagtc caggattgct ctaggacagg tggcaggatc tgaataaagg actgcaaaga |
| 961 | ctggcttcat gtccattcac aaggaccagc tctgtccttg gcaggccaat ggaacacctc |
| 1021 | ttctgccacc atgctgtgac aacccaactg acatcttcct atggaagttt gccctctcca |
| 1081 | caaaagaagt agaatgtttg cattggagct gggcatgctc tgcttcccct cagtgccccg |
| 1141 | agaatgttat ctaatgctag tcatcattaa tagctcccta cagaactttc atacagttgc |
| 1201 | acccaagttg ctgatgtgtt ctctagaata gagcaagaaa tagtaaacag aattcctttt |
| 1261 | gcctctctgt actattttcc cccaaatacc aagatttgta tgttttataa agctaatttc |
| 1321 | cttatcaaat gacatctttt aatttttaca ttaatggctt attttcaagg tacaacctga |
| 1381 | tttttttatg gacaaaaatg atgtaaaatc aaataaaa// |

Mus musculus cDNA beta-2 adrenergic receptor GenBank:
AK080241.1 Meth. Enzymol. 303, 19-44 (1999) (SEQ ID NO: 10)

| | |
|---|---|
| 1 | ggcactgcaa ggctgcttct caggcattca ggctgcggct gcaggcaccg cgagcccgga |
| 61 | gcacccacga gctgagtgtg caggacgcac cccagcacag ccacctacgg ccgctgaatg |
| 121 | aagcttccag gagtccgccc ccggccggct gcgccccgtc ggaggtgcac ccgctgagag |
| 181 | cgcctgggca ccgaaagccg gtgcgctcac ctgctaacct gccagccatg gggccacacg |
| 241 | ggaacgacag cgacttcttg ctggcaccca acggaagccg agcgccagac cacgacgtca |
| 301 | ctcaggaacg ggacgaagcg tgggttgtgg gcatggccat cctcatgtcg gttatcgtcc |
| 361 | tggccatcgt gtttggcaac gtgctggtca tcacagccat tgccaagttc gagcgactac |
| 421 | aaaccgtcac caactacttc ataatctcct tggcgtgtgc tgatctagtc atgggcctag |
| 481 | cggtggtgcc gtttggggcc agtcacatcc ttatgaaaat gtggaatttt ggcaacttct |
| 541 | ggtgcgagtt ctggacttcc attgatgtgt tgtgcgtcac agccagcatc gagaccctgt |
| 601 | gcgtgattgc agtggatcgc tatgttgcta tcacatcgcc cttcaagtac cagagcctgc |
| 661 | tgaccaagaa taaggcccga gtggtcatcc tgatggtatg gattgtatct ggccttacct |
| 721 | cctttttgcc tatccagatg cactggtacc gtgccaccca caagaaagct atcgattgtt |
| 781 | acaccgagga gacttgctgt gacttcttca cgaaccaggc ctacgccatc gcgtcctcga |
| 841 | ttgtgtcttt ctacgtgccc ctggtggtga tggtctttgt ctattcccgg gtcttccagg |
| 901 | tggccaaaag gcagctgcag aagatagaca aatctgaagg aagattccac gcccaaaacc |
| 961 | tcagccaggt ggagcaggat gggcggagcg gccacggact ccgaaggtcc tccaagttct |
| 1021 | gcttgaaaga gcacaaagcc ctcaagactt taggcatcat catgggcaca ttcaccctct |
| 1081 | gctggctgcc cttcttcatt gtcaatatcg tgcacgttat cagggacaac ctcatcccta |
| 1141 | aggaagttta catcctcctt aactggttgg gctacgtcaa ctctgccttc aatcctctta |
| 1201 | tctactgtcg gagtccagat ttcaggattg cctttcaaga gcttctgtgc cttcgcaggt |
| 1261 | cttcttcgaa aacctatggg aacggctact ctagcaatag caacggcaga acggactaca |
| 1321 | caggggagcc aaacacttgt cagctggggc aggagagaga acaggaactg ctgtgtgagg |
| 1381 | atcccccagg catggaaggc tttgtgaact gtcaaggtac tgtgcctagc cttagcgttg |
| 1441 | actcccaagg aaggaactgt agtacaaatg actcgccact gtaatacagg ctttctactc |
| 1501 | tctaagaccc ctccttgaca ggacactaac cagactattt aacttgagtg taataacttt |
| 1561 | agaataaaat tgtatagaga tttgcagaag g |

### Photolysis of Viral-Transduced SS Cells, SS Progenitor Cells and Erythroleukemia Cells (Disclosed First U.S. 60/809,553 and PCTUS 2010/0203024A1 Paragraphs [0140] to -[0148] Incorporated by Reference in Entirety)

SS erythrocytes produce an abundance of hemoglobin degradation products rendering them photosensitive. Non-enzymatic heme-iron degradation is initiated by autooxidation of hemoglobin S and randomly attacks carbon-methene bridges of the heme moiety tetrapyrrole rings. This results in a 4-10 fold increase in fluorescent heme degradation products (FHDP) such as hemichromes and protoporpohyrin IX, release of free iron and generation of 2 fold higher amounts of reactive oxygen species (ROS) than normal RBCs. Membrane-bound hemichromes produced in this process along with earlier bound hemoglobin S are targeted by activated O₂, superoxide and OFF radicals resulting in membrane injury and cell lysis.

Protoporphyrins produced during the intermediate metabolism of heme are among the most effective RBC photosensitizers. In SS cells, their progenitors and erythroleukemia cells the intracellular concentration in RBCs increases after administration of the precursor 5-aminolevulinic acid. Other RBC photosensitizers effective in this invention include but are not limited to furocoumarins, xanthene dyes, α-alkylamino-2-arylquinolinemethanol antimalarial compounds, chlorpromazine, griseofulvin, carprofen, phthalo-cyanine sulphonates, sulphonated chloro-aluminium phthalocyanine (AlPcS), chlorin e₆ (Chl-e₆), HY and the mono-sodium salt (HY-Na), haematoporphyrin derivative (HPD), PhotofrinAE (PF), haematoporphyrin (HP), and benzoporphyrin derivative monoacid ring A (BPD-MA), protoporphyrin IX.

In the claimed invention, mature SS cells, SS progenitors and erythroleukemia cells (stably transduced with BCAM/Lu) containing increased amounts of naturally produced or exogenously induced photosensitzers such as deoxyhemoglobin, denatured hemoglobin and protoporphyrin IX are infected with oncolytic viruses as described herein with virus yields of 10⁵-10¹⁰ p.f.u./ml 48 hours post infection. These cells are then exposed to visible, ultraviolet or laser light in a range or 200-900 nM for 2-60 minutes which induces a hemolysis t½ of 30-60 minutes (Grossheimer L I Photosensitization of Red Blood Cell Hemolysis: A Brief Review http://www.photobiology.com/reviews/5/index.htm (1998); Bilgin M D et al., Photochem Photobiol. 72:121-7 (2000); Grossweiner L I et al., Lasers Med Sci 13: 42-54 (1998); Fernandez J M et al., J. Photochem. Photobiol. B: Biology 37: 131-140 (1997)). The latter references and their references cited are incorporated by reference in entirety. The cells (10⁵-10¹²) are administered parenterally and localize in the tumor neovasculature where photohemolysis takes place with shedding of oncolytic virus into the surrounding tumor milieu.

Various photosensitizing agents listed above are incubated with mature SS cells, SS progenitors and erythroleukemia cells transduced with oncolytic virus ex vivo before exposure to a visible light wave source for a duration that ensures t½ hemolysis in 30-60 minutes after administration to tumor bearing hosts. In a typical regimen using Lutetium (III) texaphryrin (PCI-0123; Lu-Tex) as photosensitizer, SS cell isolated from fresh human blood from a SS homozygous patient with sickle cell anemia and diluted in pH 7.4 phosphate buffered saline (PBS) to give a light-scattering OD=2.0 at 750 nm. The cells are incubated with Lu-Tex for 90 min at 37° C. in PBS. The Lu-Tex is pre-treated by bath sonication for 30 min at 25° C. The final RBC concentration is 5.0×10⁷ for human cells. One set of irradiations is made with the unbound Lu-Tex in the external medium. In another set of irradiations the cells are centrifuged and resuspended two times to remove the unbound Lu-Tex. Spectral measurements showed that >97% of the initially bound Lu-Tex remains bound to the RBC. The cells are irradiated in a 2 cm×2 cm cylindrical cuvette with oxygen bubbling and slow stiffing while the transmission at 633 nm is monitored with a 1 mW He—Ne laser. The irradiation source is a Quantum Devices Model QBMEDXM-728 multi-element LED (730 nm maximum, 35 nm FWHM) located 3 cm from the irradiation cuvette. The on-axis incident fluence rate measured with a Newport Model 835 power meter is 63 mW cm⁻² (Bilgin et al., supra (2000)). Hemolysis is negligible during the irradiations.

One of skill recognizes that this is a model for the Lu-Tex photosensitization and that other photosensitizers are effective in initiating SS cell hemolysis. Conditions for maximum operability of these agents in the present invention may vary with each photosensitizer but not require undue experimentation. Any photosensitizing agent is useful in this invention including but not limited to 5-aminolevulinic acid, protoporphyrin IX, Texaphrin, furocoumarins, xanthene dyes, α-alkylamino-2-arylquinolinemethanol antimalarial compounds, chlorpromazine, griseofulvin, carprofen, phthalo-cyanine sulphonates, sulphonated chloro-aluminium phthalocyanine (AlPcS), chlorin e₆ (Chl-e₆), HY and the mono-sodium salt (HY-Na), haematoporphyrin derivative (HPD), PhotofrinlE (PF), haematoporphyrins (HP), and benzo-porphyrins.

In one embodiment, erythroleukemia cells stably transfected with BCAM/Lu and infected with oncolytic virus as given herein are grown in Dulbecco's modified Eagle's medium supplemented with 15% fetal calf serum (Gibco) in a humidified incubator enriched with 10% CO₂ at 37° C. supplemented with cold 5-amino levulinic acid (5-ALA) 5×10⁻⁴M and [4⁻¹⁴C] ALA (0.1/μCi ml⁻¹). The cells are subdivided twice a week by resuspension in fresh medium at a concentration of ˜5×10⁵ cells

ml⁻¹. After 8 days of culture, cells (10⁸) are harvested, washed twice in PBS (0.1 M, pH=7.2). The resuspended cells (10⁶ ml⁻¹) are irradiated for 2 min from 10 mm distance, using a ‘black-light’ source, delivering 10Wm⁻². The emission spectra of the light are in the region 320-450 nm, with a maximum at 380 nm (Malik Z et al., Br. J. Cancer 56: 589-595 (1987). At the end of light exposure the cells are immediately collected and injected into tumor bearing hosts. The administered erythroleukemia cells localize to the tumor neovasculature within 10 minutes after delivery, undergo photohemolysis 20 minutes later and shed their oncolytic viral contents into the tumor mileau. The light exposure may also be delivered to the host from an exogenous source after the administrated erythroleukemic cells have localized in tumors (usually 5-30 minutes after intravenous injection/infusion).

In an additional embodiment, protoporphyrin IX accumulation in the same erythroleukemia cells and specific cell lysis induced by exposure to 1 mM delta-aminolevulinic acid (ALA) for 2-5 hours is increased significantly by inclusion with ALA of 1,10-phenanthroline (0.75 mM), a tetrapyrrole biosynthesis modulator during the incubation in a method described by Rebeiz N et al Photochem Photobiol 44: 679-687 (1986).

Additional disclosure of this invention is provided in PCTUS0769869 published as PCTUS 2010/0203024 A1 incorporated in entirety by reference. The most relevant disclosure is given below in the paragraph

Before administration in vivo, the red cell ghosts encapsulating the desired therapeutic substance prepared by the above methods are optionally exposed to a light source in a wave length of 50-900 nanometers for 5-60 minutes designed to induce a photohemolysis W2 of 20-60 minutes. Following parenteral administration red cell ghosts localize in tumor neovasculature where they undergo photohemolysis shedding their contents into the tumor milieu. The ghosts may also be coencapsulated with small amounts of ferrous particles and hemoglobin or exposed exogenously to photosensitizers and light ex vivo as described above to ensure timely photohemolysis after the cells are administered in vivo and localized in the tumor bed.

In PCTUS 2010/0203024 A1, paragraphs [0170]-[0185], Table 4 and Example 7, paragraphs [0413-0420] contain further relevant disclosures of this invention including the methods for preparation of SSRBC ghosts and their encapsulation of numerous biologic agents and tumoricidal drugs for treatment of primary and metastatic tumors. Thesse paragraphs, Table 7 and Example 7 from PCTUS 2010/0203024 A1 are quoted verbatim below.

Sickle Cell Ghosts, SS Erythrocytes, SS Progenitors and Erythroleukemia Cells as Carriers of Tumoricidal Agents

The extremely plastic structure of the erythrocyte and the ability to remove its cytoplasmic contents and reseal the plasma membranes enable the entrapment of different macromolecules within the so-called hemoglobin free “ghost.” Combining these ghosts and a fusogen such as polyethylene glycol has permitted the introduction of a variety of macromolecules into mammalian cells (Wiberg, F C et al. Nucleic Acid Res. 11: 7287-7289 (1983); Wiberg, F C et al, Mol. Cell. Biol. 6: 653-658 (1986); Wiberg, F C et al, Exp. Cell. Res. 173: 218-227 (1987)). The mature sickled erythrocyte, SS progenitor cells and erythroleukemia cells can be modified in this way and still retain its rigid membrane structure. Thus it can be used to entrap tumoricidal agents, oncolytic viruses and plasmids encoding oncolytic viruses, toxins, toxin-antibody conjugates, therapeutic monoclonal antibodies and carry them into the tumor vasculature. Tumor killing agents introduced into the sickled erythrocyte are released locally following deposition in the tumor microcirculation. Some of the most promising agents include spores of Clostridia perfringens novyi a non-pathogenic anaerobic bacteria selectively activated in anaerobic tissue has shown tumoricidal activity in murine models (Dang et al, Proc. Natl. Acad. Sci. 98: 15155-15160 (2001), non-pathogenic Listeria monocytogenes which specifically activates tumor killing (T̂-1) cytokines and also produces a hemolysin (listerolysin) or dead but metabolically active listeria or other bacterial species that enables it to lyse the SS erythrocytes from within the cell (Brockstedt et al, Nat. Med. 11:853-60 (2005)). Modified bacteria are incorporated into the erythrocytes by fusion of the bacteria with erythrocyte membrane followed by internalization. Anaerobic spores such Clostridia novyi are encapsulated by sickled erythrocytes, SS progenitors, or erythroleukemia cells stably transfected with BCAM/Lu by the methods of Schrier S. Meth. Enzymol. 149: 261-271 (1987); Tsong T Y Methods. Enzymol. 149:248-259 (1987); Deloach J R Meth. Enzymol. 149: 235-242 (1987)); however, any other encapsulation procedure described below or in Methods in Enzymology, vol 149, Academic Press, New York, N.Y (1997) herein incorporated by reference in their entirety is useful in the present invention. Anti-tumor drugs especially those active under anaerobic conditions can be also be encapsulated in this fashion. Phage displays, exosomes, sickle cell vesicles, yeast sec vesicles expressing tumoricidal toxins or superantigens can be prepared and incorporated into mature sickled erythrocytes by fusigenic methods previously described. These cells loaded with spores are preferentially exposed to photosensitizers and a light source as described above to induce a VA cell lysis of 10-60 minutes. The are administered to tumor bearing hosts, deposit in the tumor neovasculature, undergo photolysis and release their contents in the tumor milieu.

Various types of chemotherapy can be loaded into mature sickled erythrocytes or erythrocyte ghosts preferably before administration some which have particular effectiveness in the hypoxemic micro-environment of the tumor. These include quinone-containing alkylating agents, of which mitomycin C is the prototype and nitroaromatic compounds, of which misonidazole and RB 6145 are examples. Tirapazamine is the prototype hypoxia-activated prodrug and is particularly useful. Its toxic metabolite is a highly reactive radical present at higher concentrations under hypoxia that selectively kills radio-resistant hypoxic cells in tumors. This makes the tumors much more sensitive to treatment with conventional chemotherapy and radiotherapy. An additional chemotherapeutic useful in this invention is dolostatin an anti-vascular agent that leads to vascular shutdown in tumors and traps molecules such as sickled erythrocytes with their tumoricidal loads in the tumor microvasculature. Indeed, various antineoplastic drugs such as actinomycin D, bleomycin and cytosine arabinoside are entrapped in erythrocyte ghosts by well established methods (Deloach & Barton C. Am. J. Vet. Res. 42: 1971 (1981); Deloach & Barton C. Am. J. Vet. Res. 43:2210 (1983); Lynch W S et al., Am. J. Hematol. 9: 249 (1980)). Normally, the drug is added externally and incorporated inside the erythrocyte by a passive mechanism. However, if the molecular weight of the substance to be encapsulated is greater than the cutoff of the dialysis tube, the drug is added to the erythrocytes before dialysis. If available in limited amounts the drug is incubated directly after the dialysis step with the dialyzed erythrocytes. Here, the cells are preferentially photosensitized and exposed to light ex vivo as described above and then administered to tumor bearing hosts where they localize in tumor neovasculature.

SS ghosts from mature SA, SS erythrocytes from patients with sickle cell trait or sickle cell anemia respectively are useful for encapsulation of anaerobic bacteria such as Clostridia novyi, Listeria or S. aureus because under physiologic conditions they show normal morphology whereas under the more extreme conditions of hypoxia such as the acidotic and/or hypoxemic tumor microvasculature they sickle and become adherent to the microvasculature. Once adherent to the endothelium of the tumor microcirculation, they obstruct microvasculature in a manner similar to the homozygous SS erythrocytes.

The present invention contemplates sickle cell ghosts, SS erythrocytes, their precursors, variants and erythroleukemia cells as carriers of chemotherapy, prodrugs, antitumor angiogenic therapy, tumoricidal proteins, toxins, e.g., superantigens, diphtheria, ricin, pseudomonas exotoxin A and toxin-tumor specific antibody conjugates, tumor specific antibodies, enzymes and metals such as iron and gold selectively into tumors. They can be carriers of Qdots, liposomes and nanoparticles or any other type of biocompatible particle with tumor localizing properties.

### SS Cell Encapsulation Methodology

### Collection and Washing of Erythrocytes

The methods to encapsulate drugs, enzymes or peptides are based on the property of the RBC to increase in volume when placed under conditions of reduced osmotic pressure, such as in the presence of a hypotonic solution. The hypotonic encapsulation method of Deloach Jr et al. Am. J. Vet Res. 42: 667-671 (1981) considered to be representative of the field and preserves the biochemical and physiological characteristics of the erythrocytes and the highest percentage of encapsulation.

Carrier erythrocytes may be prepared from human blood and blood of different animal species such as rat, mouse, rabbit, dog, etc. Blood is taken from patients with sickle cell anemia with homozygous SS hemoglobin and the erythrocytes collected using an appropriate anticoagulant such as EDTA or a mixture of citrate, phosphate and dextrose (CPD) because it best preserves the properties of red blood cells although some use heparin (1000 IE/10 ml blood). Erythrocytes are separated from serum and buffy coat by centrifugation at room temperature and washed 4 times in via centrifugation (530 g, 15 min, 4° C.) with isotonic solutions usually Hanks-PBS buffer to remove other blood components. It is also possible to achieve a good washing with a plasma separator.

### Dialysis of Erythrocytes Against Hypotonic Buffer

This step allows substances to enter the red cells by an increase in porosity due to the hypotonic environment. Washed packed erythrocytes (hematocrit 50-90%; 5 to 10 ml) are placed inside a dialysis bag. The substance to be encapsulated is added either to the actual suspension of erythrocytes, adjusting the final hematocrit of the suspension, or dissolved in the external dialysis buffer. Dialysis is carried out with an appropriate hypotonic buffer at 48° C., pH of 7.4 and continued for various periods. Single dialysis membranes with a molecular cutoff of 3.4-14 kDa, are useful although two types of membrane with different molecular weight cutoffs are recognized in the art. Tonicity is restored by addition of sufficient quantity of 154 mM NaCl to bring the osmolality up to 300 mOsm. By raising the salt concentration to its original level, the RBC pores close, the RBCs reassume their normal biconcave shape and the substance remains encapsulated inside the cells at a suitable concentration. Nonentrapped substances are washed out and the loaded RBCs are ready to be used as carriers for the delivery of the encapsulated drugs (Rossi et al., Expert Opin. Drug Deliv. 2: 311-322 (2005)).

The art recognizes that composition and osmolality of the buffers may vary depending on the animal species employed and the substance to be encapsulated exemplified in Table 2 of Rossi et al. Expert Opin. Drug Deliv. 2:311-322 (2005). For human erythrocytes, the osmolalities of the hypotonic buffers vary from 26 to 220 mOsm/kg based on the substance to be encapsulated (see Table 3 of Rossi et al. Expert Opin. Drug Deliv. 2: 311-322 (2005)). The duration of the dialysis for human erythrocytes ranges between 20 and 180 mM Additionally, the volume ratio (v/v) between the erythrocyte suspension and the dialysis buffer of 1:50 is effective. Automated systems with dialysate flow rates ranging from 15-19 ml/min to 20-60 ml/min are useful. Several buffers are used to wash the erythrocytes before and after dialysis. These include but are not limited to (i) 154 mM NaCl; (ii) 15 mM Na₂HPO (pH 7.0), 10 nM glucose, 144 mM NaCl (phosphate butter); (iii) 1.5 mM NaCl pH 7.0), 10 mM glucose, 144 mM NaCl (phosphate-MgCl₂ buffer); (iv) 5 mM Na₂HPO₄, pH 7.0, 0-5 mM CaCl₂, 10 mM glucose, 154 mM NaCl (phosphate-CaCl₂ buffer; (v) 10 mM Tris HCl (pH 7.0), 10 nM glucose, 144 mM NaCl: and (vi) 154 mM NaCl, 5 mM MgCl₂ (NaCl—MgCl₂ buffer).

### Resealing Step

The resealing step produces entrapment and encapsulation of a drug, enzyme, antibody, polypeptide by closing the RBC pores. The dialysis bag containing the erythrocyte suspension is transferred to an isotonic or hypertonic buffer isotonic buffer such as Hanks-PBS for 10 mM at 37° C., or a highly hypertonic buffer is added at a proportion of 0.1:1 (v/v) directly to the erythrocyte suspension. The buffer compositions employed in the resealing step are given in Table 2 of Gutierrez Millan C et al. Blood Cells, Molecules, Diseases 33: 132-140 (2004). After resealing, the erythrocytes are washed several times with an isotonic buffer at 48° C. and then resuspended in plasma for later reinjection. Washing with hypotonic buffers leads to the removal of the most fragile carrier cells.

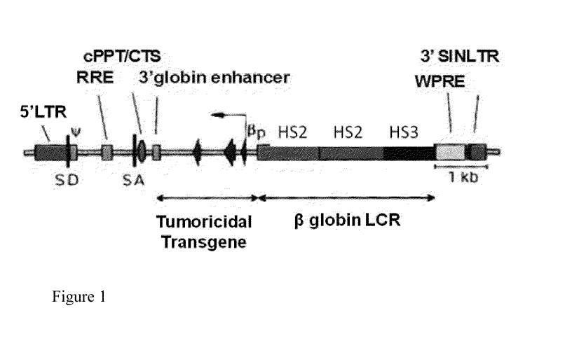

Quantitative range of reagents used in the performance of encapsulation of various molecules within intact human RBCs is a summarized in Table 4 (FIG. 1, from Gutierrez Millan C et al. Blood Cells Molecules, Diseases 33: 132-140 (2004). Technical modifications may vary with the physical properties of the species of molecule to be encapsulated but are well within the skill of the ordinary scientist

Encapsulation Protocol is Given in Example 7 (Provided Below).

Before administration in vivo, the red cell ghosts encapsulating the desired therapeutic substance prepared by the above methods are optionally exposed to a light source in a wave length of 50-900 nanometers for 5-60 minutes designed to induce a photohemolysis W2 of 20-60 minutes. Following parenteral administration red cell ghosts localize in tumor neovasculature where they undergo photohemolysis shedding their contents into the tumor milieu. The ghosts may also be coencapsulated with small amounts of ferrous particles and hemoglobin or exposed exogenously to photosensitizers and light ex vivo as described above to ensure timely photohemolysis after the cells are administered in vivo and localized in the tumor bed.

Vesicles from sickled erythrocytes are shed from the parent cells. They contain membrane phospholipids which are similar to the parent cells but are depleted of spectrin. They also demonstrate that a shortened Russell's viper venom clotting time by 55% to 70% of control values and become more rigid under acid pH conditions. Rigid sickle cell vesicles induce hypercoagulability. Vesicles shed from immature or mature sickled erythrocytes are capable of localizing to tumor microvascular sites where they bind and induce an anti-tumor effect.

Vesicles are prepared and isolated as follows: Blood is obtained from patients with homozygous sickle cell anemia. The PCV range is 20-30%, reticulocyte range is 8-27%, fetal hemoglobin range is 25-13% and endogenous level of ISCs is 2-8%. Blood is collected in heparin and the red cells are separated by centrifugation and washed three times with 0.9% saline. Cells are incubated at 37° C. and 10% PCV in Krebs-Ringer solutions in which the normal bicarbonate buffer is replaced by 20 mM Hepes-NaOH buffer and which contains either 1 mM CaCl₂ or 1 mM EGTA. All solutions contain penicillin (200 U/ml) and streptomycin sulphate (100 μg/ml). Control samples of normal erythrocytes are incubated in parallel with the sickle cells. Incubations of 10 ml aliquots are conducted in either 100% N₂ or in room air for various periods in a shaking water bath (100 oscillations per mm) N₂ overlaying is obtained by allowing specimens to equilibrate for 45 mm in a sealed glove box (Gallenkamp) which was flushed with 100% N₂. Residual oxygen tension in the sealed box is less than 1 mmHg. The percentage of irreversibly sickled cells is determined by counting. 1000 cells after oxygenation in room air for 30 mm and fixation in buffered saline (130 mM NaCl, 20 mM sodium phosphate, pH 74) containing 2% glutaraldehyde. Cells whose length is greater than twice the width and which possessed one or more pointed extremities under oxygenated conditions are considered to be irreversibly sickled. After various periods of incubation, cells are sedimented at 500 g for 5 mm and microvesicles) are isolated from the supernatant solution by centrifugation at 15,000 g for 15 mm. The microvesicles form a firm bright red pellet sometimes overlain by a pink, flocculent pellet of ghosts (in those cases where lysis was evident) which is removed by aspiration.

Quantitation of microvesicles is achieved by resuspension of the red pellet in 1 ml of 05% Triton X followed by measurement of the optical density of the clear solution at 550 nm Optical density measurements at 550 nm give results that are relatively the same as measurements of phospholipid and cholesterol content in the microvesicles. Cell lysis is determined by measurement of the optical density at 550 nm of the clear supernatant solution remaining after sedimentation of the microvesicles. Larger samples of microvesicles for biochemical and morphological analysis are prepared from both sickle and normal cells following incubation of up to 100 ml of cell suspension at 37° C. for 24 h in the absence or presence of Ca₂⁺⁺. Ghosts are prepared from sickle cells after various periods of incubation. The cells are lysed and the ghosts washed in 10 mM Tris HCl buffer, pH 73, containing 0₂ mM EGTA.

### Example 7

Methods for SS cell encapsulation, optionally incorporating ferrous molecules are described below (G. L. Dale, et al, Biochem. Med. 18, 220 (1977); DeLoach J R & Sprandel U (eds.), “Red Blood Cells as Carriers for Drugs.” Karger-Verlag, Basel, Switzerland, (1985): Zimmermann U. et al, Biochim. Biophys. Acta 436: 460 (1976); DeLoach & Ihler G. Biochim Biophys Acta 496: 136 (1977)) which are herein incorporated by reference.

Materials

Buffer I (isosmotic): 150 mM NaCl, 5 mM K₂HPO₄/KH₂PO₄, pH 7.4.
Buffer II (isosmotic): like buffer 1, in addition 10 mM glucose, 5 mM adenosine, 1 mM MgCl₂.
Buffer III (hyposmotic): 5 mM K₂HPO₄/KH₂PO₄, pH 7.4.

### Preparation of Erythrocyte Ghosts

Erythrocyte ghosts are prepared by a hypotonic dialysis procedure with best results obtained after standardization of the following parameters. Washed red blood cells are placed into dialysis tubing. Then a solution of buffer I and (optionally) the ferrofluids to be entrapped (25% ferrofluids in buffer I) is added. The hematocrit is 75% (three volume units of red blood cells and one volume unit of buffer I-fer-rofluids solution). The erythrocytes are then dialyzed against hyposmotic buffer III for 75 mM at 4° C. After entrapment, cells are resealed by dialysis against isosmotic buffer II for 60 min at 37° C. After preparation, the resealed cells are washed again four times by centrifugation in isosmotic buffer II (530 g, 20 mM at 20° C.).

### Ferrofluids (Optional)

Ferrofluids are permanent, colloidal suspensions of magnetite (Fe₃0₄), produced and delivered by Ferrofluidics Corporation in Nashua, N.H. The only kind of ferrofluids used in these experiments are type BIO-I (catalog no. EMG 1111), a water-based ferrofluid preparation that contains no organic or inorganic detergents. The properties of BIO-I are: magnetic saturation, 200 G; density, 1.18 g/ml; viscosity, 1-10 cP, 27° C.; vapor pressure, 100° C., 760 mmHg; initial susceptibility, 0.6; magnetite particles, 25 vol % of the suspension; particle size, 8-20 nm, average 18 nm

An additional method for preparation of sickle cell ghosts is described by Bax et al, Clin Sci. 96: 171-178(1999)

### Blood Preparation

Forty milliliters of blood are collected from patients with homozygous SS sickle cell anemia and placed into two tubes containing 4 ml of anticoagulant citrate phosphate dextrose or 200 units of heparin. The blood samples are centrifuged for 10 mM and the supernatant plasma is removed and kept for later use; the buffy coat is discarded. The erythrocytes are washed twice in cold (4° C.) isoosmotic PBS, pH 7.4 (2.68 mM/1 KCl, 1.47 mM/1 KH₂PO₄, 136.89 mM/1 NaCl, 8.10 nM/1 Na₂HP0₄), and centrifuged for 10 min

### Carrier Erythrocyte Preparation

Carrier erythrocytes are prepared using a hypo-osmotic dialysis technique. Washed and packed fresh erythrocytes (10.5 ml) are mixed with 4.5 ml of cold PBS. Five milliliters of this cell suspension are placed into each of three dialysis bags (molecular mass cut-off of 12,000 Da, Medicell International Ltd, London, U.K.) sealed at both ends with clips. Each dialysis bag is placed in a container and supported firmly by wedging the dialysis clips against the container side. Dialysis is against 150 ml of hypo-osmotic phosphate buffer, pH7.4 (5 mM/1 KH₂P0₄, 5 mM/1 K₂HP0₄), at 4° C. in a refrigerated incubator with rotation at 6 rev./min Macromolecule entrapment can be increased by doubling the hypo-osmotic dialysis time to 180 min We therefore dialyzed the erythrocytes for 90 min to ascertain that cell survival in vivo is not adversely affected by an extended hypo-osmotic dialysis period. The lysed erythrocytes are resealed by transferring the dialysis bags to containers holding 150 ml of PBS supplemented with 5 mM/1 adenosine, 5 mM/1 glucose and 5 mM/1 MgCl₂, and continuing rotation at 6 rev/min in the incubator at 37° C. for 60 min Carrier erythrocytes are washed three times in 3 volumes of supplemented PBS with centrifugation at 100 g for 15 min and finally pooled.

TABLE 2
Range of Conditions Used for Encapsulation of Human Erythrocytes

| | Buffer | Temperature | Time range |
|---|---|---|---|
| Washing | isotonic | 4° C. | 5-15 min |

| | | | Dialysis Time | | Volume ratio | Hematocrit |
|---|---|---|---|---|---|---|
| | Buffer | Temperature | range | pH | range | range |
| Hypotonic | 26-220 mOsm/kg | 4° C. | 20-180 min | 7.4 | 1/10-1/300 | 50-90% |
| Dialysis | | | | | | |

| | | | Annealing | |
|---|---|---|---|---|
| | Buffer | Temperature | Time range | pH |
| Annealing | isotonic | 25-39° C. | 10-30 min | 7.4 |

| | Volume ratio | | Resealing time | |
|---|---|---|---|---|
| | range | Temperature | range | pH |
| Resealing | 1/10-1/30 | 4-39° C. | 5-60 min | 7.4 |

| | Buffer | Temperature | Time range |
|---|---|---|---|
| Washing | isotonic | 4° C. | 5-15 min |

The compositions of the claimed invention are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

The compositions are also be useful for the prevention of metastases from the tumors described above either when used alone or in combination with radiotherapeutic, photodynamic, and/or chemotherapeutic treatments conventionally administered to patients for treating disorders, including angiogenic disorders. Treatment of a tumor with surgery, photodynamic therapy, radiation and/or chemotherapy is followed by administration of the compositions to extend the dormancy of micrometastases and to stabilize and inhibit the growth of any residual primary tumor or metastases. The compositions can be administered before, during, or after radiotherapy; before, during, or after chemotherapy; and/or before, during, or after photodynamic therapy.

The present invention contemplates that erythrocytes or erythroblasts from patients with any form of sickle hemoglobinopathy are useful. These include erythrocytes or erythroblasts from hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Indeed, any erythrocyte or erythroblasts with or without sickle hemoglobin expressing receptors capable of binding to tumor neovasculature are useful in the inventions described herein. Particularly useful are those cells which express hemoglobin S in combination with other types of hemoglobin. Both mature and nucleated forms of these cells are useful. In addition, the present invention contemplates that normal or leukemic erythrocytes or their nucleated progenitors transduced with hemoglobin genes from patients with hemoglobinopathies to produce a cell that behaves substantially like an SS or SA erythrocyte or erythrocyte precursor is useful. The present invention also contemplates that normal or sickle erythrocytes or sickle variants, e.g., HbSC cells, and nucleated progenitors which are upregulated by hormones, cytokines, biologically active agents, drugs, chemical or physical treatments to express adhesive properties or to enhance expression of adhesive properties are also useful in this invention.

Additional post-filing disclosure of this invention is depicted in Choe, Terman et al., Drug-loaded sickle cells programmed ex vivo for delayed hemolysis target hypoxic tumor microvessels and augment tumor drug delivery Journal of Controlled Release 171: 184-192 (2013).

Mature SS Erythrocytes, SS Erythroblasts with a Porphyric Phenotype and Porphyria Cells with an SS Phenotype

In an additional embodiment, the gene encoding aminolevulinic acid deamidase is silenced via a siRNA in an SS hematopoietic progenitor cell; alternatively, the SS globin gene is inserted into erythroid progenitor cells from patients with porphyria cutanea tarda, erythrogenic porphyria or acute intermittent porphyria; cells from variants of these diseases or other diseases which over-produce photosensitizing porphyrins are also useful in this invention. These cells produce an abundance of photosensitizing porphyrins including protoporphyrin IX that are activated by visible light resulting in photooxidative hemolysis.

In the present invention, these porphyric progenitor cells are transduced by oncolytic virus or nucleic acids encoding tumor/angio-specific immunoglobulins (e.g., anti-VEGF agents). Likewise, a drug resistant population may be produced by exposure to antitumor agents in vivo as described above. These porphyric progenitor cells (10⁴-10¹¹) are optionally exposed to visible light at wave lengths of 200-900 nM for 10 minutes and then administered parenterally to tumor-bearing hosts where they deposit in the tumor neovasculature. The cell undergoes photohemolysis within 30 minute after parenteral administration with consequent shedding of the oncolytic virus, antitumor drug or tumor-specific/neovascular (VEGF)-specific monoclonal antibody into the tumor mileau. The kinetics of cell injury as a function of light exposure (wave length and duration) are determined beforehand so that the ex vivo light-induced photo-oxidation reaches a t½ 20-60 minutes after administration when the cells are deposited in the tumor neovasculature.

The present invention contemplates that erythrocytes or erythroblasts from patients with any form of sickle hemoglobinopathy are useful. These include erythrocytes or erythroblasts from hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. Indeed, any erythrocyte or erythroblasts with or without sickle hemoglobin expressing receptors capable of binding to tumor neovasculature are useful in the inventions described herein. Particularly useful are those cells which express hemoglobin S in combination with other types of hemoglobin. Both mature and nucleated forms of these cells are useful. In addition, the present invention contemplates that normal or leukemic erythrocytes or their nucleated progenitors transduced with hemoglobin genes from patients with hemoglobinopathies to produce a cell that behaves substantially like an SS or SA erythrocyte or erythrocyte precursor is useful. The present invention also contemplates that normal or sickle erythrocytes or sickle variants, e.g., HbSC cells, and nucleated progenitors which are upregulated by hormones, cytokines, biologically active agents, drugs, chemical or physical treatments to express adhesive properties or to enhance expression of adhesive properties are also useful in this invention.

SS erythroid progenitor cells or mature SS cells (10⁵-10¹¹) synthesizing a tumoricidal transgene preferably a superantigen such as SEG or SEI or a superantigen-tumor specific monoclonal antibody fusion protein/conjugate or siRNAs, miRNAs, shRNAs are administered parenterally every other day for up to 6 days preferably intravenously to tumor bearing mice in protocols described in detail in the section on tumor models and in humans Examples 5 and 6. These cells localize in tumor sites where they occlude tumor neovessels, and secrete their tumoricidal protein into the tumor parenchyma. These cells are tested for anti-tumor activity in the animal tumor models provided in the section below on “Animal Tumor Models.”

### Use of Constitutive Multi-Drug Resistant Genes in SS Erythroid Precursors and T Cells for Delivery of Chemotherapy to Tumors

The instant invention envisions chemotherapeutically resistant T cells and SS progenitor cells as useful in the present invention. Naïve T cells and SS progenitor cells contain the multi-drug resistance gene, MDR1 encoding an ATP-dependent plasma membrane efflux pump, P-glycoprotein (P-gp) and ABG transporters. The P-gp extrudes a broad range of hydrophobic drugs from cells, including vinca alkaloids, anthracyclines, epipodophyllotoxins, colchicine, actinomycin D and taxotere. Exposure to cytolytic chemotherapeutic agents during the loading and extrusion process does not injure the SS progenitor cells and in particular spares their genetic and secretory apparatus.

Chemotherapeutic resistance in SS progenitor cells occurs by uninterrupted or periodic exposure to the chemotherapeutic for periods ranging from 1 hour to 2 weeks. Once the chemotherapeutic is removed from the media the drug is actively expelled from the T cell or SS progenitor cell 4 to 10 times faster than a cell which is not drug resistant. Thus shortly after removal of the chemotherapeutic agent from the media the cells are collected and administered to the patient. Within minutes after injection T cells or SS progenitor cells become entrapped in the tumor endothelium and actively extrude as much as 50% of their intracellular chemotherapeutic content into the tumor parenchyma.

Chemotherapeutic resistance is also be induced in the genetically engineered T cells as described in the above section. In addition, Naïve T cells or SS progenitor cells or T cells or SS progenitors that have are transduced as described above with the vectors encoding tumoricidal molecules such as superantigens and homologues, staphylococcal enterotoxin fragments, superantigen and superantigen homologue-tumor specific antibody conjugates, pseudomonas exotoxins, diphtheria, ricin etc are useful in this invention. Thus when the engineered T cell or SS progenitor cell binds or adheres to the tumor cell or tumor endothelium it actively extrudes its chemotherapeutic and secretes a tumoricidal toxin. In addition, as the SS progenitor cell undergoes oxidant-induced lysis and releases constitutive heme. In the latter case, all three tumoricidal molecules work synergistically to kill the tumor.

Constitutive MDR1 can be combined with other drug resistance genes transduced into the SS progenitor cells in order to broaden the spectrum of drugs that are extruded from the cell. For instance, the wild-type version of human MDR1 (containing Gly at position 185) confers preferential resistance against vinblastine while the mutant with Val at position 185 confers resistance to colchicine).

T cell or SS progenitor cells can also be loaded with cytotoxic drugs in prodrug form. Loading of the cells with chemotherapeutic drugs or prodrugs is accomplished by osmotic diffusion or electroporation and other methods well established in the art. The drug metabolizing cytochrome P450s (CYPs) notably 1A, IB, 2C, 3A, 2D subfamily members have been identified in a wide range of human cancers. Individual tumor types have distinct P450 profiles as studied by detection of P450 activity, identification of immunoreactive CYP protein and detection of CYP mRNA. Selected P450s, especially CYP1B1, are overexpressed in tumours including cancers of the lung, breast, liver, gastrointestinal tract, prostate, bladder. Several prodrug anti-tumour agents have been identified as P450 substrates. Those in clinical use include prodrug alkylating agents cyclophosphamide, ifosphamide, dacarbazine, procarbazine, Tegafur, a prodrug fluoropyrimidine, methoxymorphylinodoxorubicin, a metabolically activated anthracycline, as well as flutamide and tamoxifen, two non-steroidal hormone receptor antagonists that are significantly more active following CYP-hydroxylation. New agents selectively dependent on tumor CYP activation include 2-(4-aminophenyl) benzothiazoles exclusively in CYP1A1 inducible tumors. Some CYPs operate most effectively under hypoxemic conditions. Indeed, bioreductive prodrugs such as the indolequinone AQ4N (a CYP3A substrate) and MUP 98176 are activated to cytotoxic metabolites specifically in hypoxic tumor regions after bioreduction.

In vivo, bioreductive prodrugs are transported out of the T cell or SS progenitor cells and taken up by surrounding tumor cells. Tumor cells overexpress oxyreductase systems cytochrome P450 enzymes and/or its congeners, oxidize prodrugs to their reduced and active state resulting in oncolysis. Several of these active metabolites are significantly more cytotoxic under hypoxemic conditions within tumor cells.

As an example, engineered or naïve T cells and engineered or naïve T cells SS erythroid precursors are rendered drug resistant after continuous exposure to small doses of Adriamycin for 120 hours after which Adriamycin is completely effluxed from the cell over a period of 30 minutes (Yanovich et al., Cancer Res. 44: 4499-4505 (1989); Koshkin V, PLOS One 7(7) e4 1368 (2012) In the present invention T cells or SS hematopoietic progenitor cells are exposed to various forms of chemotherapy and the optimal time course for development of drug resistance and release following discontinuation of drug is determined. After induction of drug resistance, these cells (10⁸-10¹³) are infused into tumor-bearing hosts where they deposit in tumors and actively expel their drug directly into the tumor parenchyma.

Representative methodology for measurement of accumulation and efflux of drugs and probes in T cells and hematopoietic progenitor cells is as follows: Cellular contents of fluorescent MDR probes were determined by flow cytometry (BD FACSCanto II flow cytometer, BD Biosciences, Franklin Lakes, N.J.) in a medium comprised of 140 mM NaCl, 3 mM KCl, 10 mM Na₂HPO₄, 2 mM KH₂PO₄, 2 mM CaCl₂, 1 mM MgCl₂, and 10 mM glucose. After being loaded with the labeled drug or drug surrogate probe (1-5 μM, 30-45 min, 37° C.), cells (approximately 10⁶ cells/mL) are sedimented, washed, resuspended in fresh medium (containing 5 mM PI), and examined for intracellular probe content using a standard argon-ion laser emitting at 488-nm for fluorescence excitation and a 530/30 nm band-pass emission filter for the fluorescence detection of

rhodamine 123, calcein, and fluorescein and a 585/42 nanometer band pass emission filter for the detection of PI. To evaluate the accumulation of mitoxantrone, the samples are excited with a 635-nm
red diode laser, and a 680/32 nm band pass emission filter was used to detect fluorescence. For the quantitative kinetic MDR study, the progression of dye efflux was monitored by flow cytometric repetitive sampling of cell suspensions at appropriate time points.

In a second method, intracellular accumulation and retention of daunorubicin in K562-S and K562-R cells is determined as follows, Cells are initially incubated with daunorubicin (0.5 μg/ml) in dextrose-PBS in a shaking water bath at 37° C.; after 2 h, the daunorubicin containing medium is removed and the cells are resuspended in dextrose-PBS at 37° C. in a shaking water both for an additional 30 min Samples from each cell line are obtained for high-performance liquid chromatography analysis after the initial 2-h incubation with daunorubicin (initial point), and at different times following incubation in drug-free medium. The percentage of intracellular daunorubicin retained after 5, 10, 15, 30, 45, 60, 75, 90 and 120 min of incubation in drug-free medium is determined.

These engineered or naïve T cells or SS progenitor cells are used as a cell line. Engineered T cells include the chimeric T cells described above. They also include naïve T cells or SS progenitor cells or T cells and SS progenitor cells transduced with tumoricidal molecules such as superantigens or superantigen homologues and superantigen (or superantigen homologue)-tumor specific antibody conjugates, superantigen fragments, toxins such as pseudomonas exotoxin, diphtheria, ricin, shiga toxins, Panton Valentine leucocidins as described above.

Optionally, ex vivo exposure of both drug resistant and non-drug resistant cells (incubated with a tumoricidal drug for 8-120 hours) to light radiation (200-900 nM) that induces a hemolysis t½ of 20-60 minutes is useful to ensure release of the drug from the T cells, SS progenitor cells once they have deposited in the tumor vasculature. In this way chemotherapy can be specifically targeted to and concentrated in the tumor.

Likewise, nucleic acids encoding monoclonal antibodies specific for epitopes expressed on tumor cells, tumor parenchyma or tumor vasculature can be transfected into the T cells or SS progenitor or erythroleukemia cells using methods described herein. An example of one such monoclonal antibody is Avastin specific for VEGF receptors on tumor endothelium. T cells or SS progenitor cells localized in the tumor vasculature release the VEGF-specific monoclonal antibodies into the tumor mileau. The tumor neovasculature is within easy reach of the recombinant antibodies. In this way, anti-angiogenic therapy such anti-VEGF is concentrated at the site of its cognate ligand in the tumor neovasculature, producing an increase in the therapeutic index of the drug and reduction in its systemic side effects.

### Growth Resistance of the Arginine Deficient B16 Melanoma in the Oxidative Vascular Microenvironment of SS Mice.

The present invention contemplates tumors deficient in arginine are especially susceptible killing in oxidative vascular microenvironment of patients with sickle cell disease. Humans and mice with sickle cell anemia exhibit a hypoxic and highly pro-oxidative vascular microvasculature that initiates and promotes sickling/vaso-occlusion. Such a pro-oxidative vascular microenvironment is predictably inimical to tumor neoangiogenesis. The prevailing thought is that the pro-oxidative vasculature in SS mice is caused by reduced NO bioavailability. The latter is largely attributable to underlying chronic hemolysis resulting in spillage of SSRBC arginase that cleaves circulating arginine. NO consumption by heme and heme products released from hemolyzed SSRBCs further diminishes NO levels. Moreover, NO is consumed in the union with locally generated superoxide producing perinitrite, a powerful vascular oxidant that enhances the pro-oxidative state in the vascular mileau. The B16 melanoma is arginine deficient due to deletion of a gene encoding the enzyme argininosuccinate synthase (ArgSS). The latter catalyzes the conversion of citrulline to argininosuccinate in the intracellular arginine synthetic pathway. Such ArgSS deficiency is also known to impair iNOS generation via decoupling of NO synthase. We tested whether arginine deficient B16 melanoma could grow in the pro-oxidative vascular microenvironment of SS mice using as a control the Lewis lung carcinoma, which has an intact arginine synthetic pathway.

For this purpose, we used the Townes murine SS model to test the outgrowth outgrowth of a B16 melanoma. B16 melanoma tumor cells (10⁵) were implanted subcutaneously in these SS mice and tumor outgrowth was compared to that of B16 melanoma cells similarly implanted in hemoglobin AA mice. Our results show that after implantation of 10⁵ tumor cells, B16 flank tumors grew in only 13% SS mice (4 of 30) of SS mice contrasted with 100% tumor outgrowth in AA mice, SA mice and C C57BL/6 mice (p<0.0001). Lewis lung carcinoma similarly implanted grew in 100% (10 of 10) of SS, SA, AA and C57BL/6 mice. It is therefore evident that ArgSS deficiency in the B16 melanoma cells combined with constitutive arginine deficiency in the SS tumor microvasculature are major factors in the reduced outgrowth of B16 melanoma relative to LLC. In confirmation, we use lenti/shRNA to knock-down or lenti/CRISPR-Cas to knockout the ArgSS gene in LLC cells and a lentiviral EF-1 alpha/ArgSS construct to over-express ArgSS in melanoma cells. For in vivo studies, the genetically modified tumor cells (10⁶ in 100 μL) are implanted in the right flank of SS, AA, SA and C57B1/6 mice using 10 mice per group and tumors measured three times weekly thereafter. Results show that LLC cells depleted of the ArgSS gene show no tumor outgrowth whereas B16 melanoma cells reconstituted with the ArgSS gene demonstrate robust tumor outgrowth (p=0.0001). We validate the knockdown/knockout and over-expression of the ArgSS gene in each case via Western analysis of the cell lysates. We further demonstrate that knockin B16 melanoma cells transfectants produce NO and whereas knockout LLC knockout cells do not in response to both cytokine and LPS stimuli using well-established methods.

SSEPCs, SSiPPSCs and SSRBCs for Delivery of Oncotropic Viruses after Introduction of Double Stranded RNA Virus Infected Cells.

The present invention contemplates that SSEPCs, SSiPPSCs or SSRBCs which localize in tumor vasculature are used to carry and deliver an oncotropic virus into the tumor mileau. Most viruses possess double- or single-stranded RNA (ssRNA) genomes and produce long dsRNA helices during transcription and replication; the remainder of viruses have DNA genomes and typically produce long dsRNA via symmetrical transcription. Once the tumor cell is transduced by the oncotropic virus, SSEPCs or SSiPPSCs or SSRBCs loaded are used to deliver a bifunctional chimeric protein to the tumor cells. One domain of the chimeric protein binds to viral dsRNA and a second domain binds to a procaspase-binding domain or a procaspase. Apoptosis ensues when two or more of these chimeric molecules crosslink on the same dsRNA. If viral dsRNA is present inside a cell, the chimeric molecule binds to the dsRNA and induce apoptosis of that cell. If viral dsRNA is not present inside the cell, the chimeric molecule will not crosslink and apoptosis will not occur.

The structure of the chimeric protein is provided in FIG. 1 of Rider et al., PloS One 6(7):e22572. The functional molecules at the dsRNA and caspase binding sites are provided in U.S. Pat. No. 7,566,694 B2 Importantly, the dsRNA detection domain of the chimeric molecule comprises a PKR1-181, PKR1-181 with dsRBM 1 (NTE3L), dsRBM 2 (CTE3L), or dsRBM 1 and 2 (26E3L) replaced by the dsRNA binding motif from poxvirus E3L, and RNaseL1-335 (which binds to 2-5A produced by endogenous cellular 2-5A synthetases in response to viral dsRNA). The apoptosis induction domain comprises FADD1-90 Death Effector Domain (DED, which binds to procaspase 8), Apaf-11-97 caspase recruitment domain (CARD, which binds to procaspase 9), and murine Apaf-11-97 (mApaf) CARD Importantly, it has been shown that these chimeric molecules are effective against viruses with DNA, dsRNA, positivesense ssRNA, and negative-sense ssRNA genomes; enveloped and non-enveloped viruses; viruses that replicate in the cytoplasm and viruses that replicate in the nucleus; human, bat, and rodent viruses; and viruses that use a variety of cellular receptors. The list below includes but is not limited to oncotropic viruses that are useful in this invention as a source of dsRNA Notably, however, because the SSEPCs and SSRBCs can selectively carry viruses to the tumor mileau, non-oncotropic viruses detained in Rider et al., supra (2011) and U.S. Pat. No. 7,566,694 B2 are also useful.
i. Adenovirus: adenovirus d/1520, adenovirus CN706, adenovirus Ad5-CD/tk-rep, ONYX-015, AdΔ24, Ad5-yCD/mutTKSR39rep-hNIS, Ad-ΔE1B19/55, hNIS, d1309, d1704(ΔE3gp19kD), E1B55kD-deleted adenovirus, Ad-ΔE1B19/55, Ad5-yCD/mutTKSR39rep-ADP, CV706, CV787 adenovirus, OBP-301, AdΔ24-p53, AdΔ24RGD CRAd-S-pk7, AdAM6, Ad-ΔE1B55
ii. Herpes simplex virus: Herpes simplex G207, Herpes simplex NV1020, NV1066, NV1023, rRp450, HSV-1, HSV-1716, R3616 (inactivated γ34.5)
iii. Vaccinia virus wild type plus GmCSF, vvDD-SSTR2, GLV-1h68, JX-963, JX-929, JX-594
iv. Newcastle Disease virus 73-T
v. Autonomous parvovirus H-1 parvovirus wild type
vi. Reovirus: Reolysin;
vii. Measles virus
viii. Vesicular stomatitis virus.

Methods for incorporation of oncolytic viruses/genome or the chimeric gene into the lentiviral β-globin vector and transduction of SSEPCs or iPPSCs are provided above. These cells along with SSRBCs are used to treat established tumors as disclosed in Tumor Models and Example 3.

Therapeutically Useful Sickle Erythrocytes are Obtained from Patients with Sickle Cell Anemia, Sickle Cell Trait and Sickle Cell Variants

For SS erythrocytes, sickling begins at PO₂40-50 mmHg and is greatest when PO₂<20. This occurs in organs with sluggish circulation, high oxygen extraction, localized hypoxia and low pH such as the renal medulla and spleen and bone marrow. The likelihood of erythrocyte sickling by hemoglobin SS variants is related to amount of Hgb S present, e.g., Hgb S: 70-98%. Hgb SA: 10-40%, Hgb SC: 50-60%.

Sickle trait (SA hemoglobin) affects approximately 8% of the black population in the United States or approximately 2.7 million individuals. The incidence is higher in tropical Africa and approaches 40% in some regions. Patients with sickle trait are heterozygous for the sickle cell hemoglobin gene, and less than 50% of the hemoglobin in each cell is hemoglobin-S. Polymerization of deoxy-hemoglobin in erythrocytes from patients with SA hemoglobin can occur under certain conditions and transform silent sickle cell trait into a syndrome resembling sickle cell disease with vaso-occlusion. In particular, sustained exercise and high altitude conditions cause tissue hypoxia, acidosis, dehydration, hyperosmolality, hypothermia can causing splenic infarction, exertional heat illness (exertional rhabdomyolysis, heat stroke, or renal failure) or idiopathic sudden death. Because of their proclivity to sickle and aggregate in hypoxic tissues, erythrocytes with SA hemoglobin are useful in the present invention.

Milosevic et al., Gynecologic Oncology 83, 428-431 (2001)) showed that erythrocytes from patients with sickle trait may sickle in the microvasculature of solid tumors and contribute to reduced blood flow and the development of hypoxia. Hypoxia is a strong independent prognostic factor in patients with cervix cancer. While this reference did not disclose the use of erythrocytes from patients with sickle cell trait for therapy of cancer, the skilled scientist would recognize that such cells can collect and aggregate under the hypoxic conditions within tumors in a fashion similar to homozygous SS sickle cell anemia. Similarly, under hypoxic conditions hemoglobins in erythrocytes from patients with other SS variants such hemoglobin SC, hemoglobin Antilles are known to polymerize leading to sickling and aggregation. Thus this population of cells is also considered to be useful therapeutically and may be safe for transfusion since they do not sickle only under hypoxic conditions such as those encountered in tumors and not under normal physiologic conditions.

Erythrocytes with SC Hemoglobin

The coinheritance of HbS and HbSC results in a clinically significant sickling disorder similar to that of sickle cell disease (HbSS). HbSC disease is usually considered less severe than Hb SS disease however, some individuals manifest a condition equal in severity. HbSC disease exhibits combined symptomatology of both Hb S and Hb C diseases independently.

Like SS disease, SC erythrocytes sickle under hypoxic conditions causing vaso-occlusion in ischemic tissues resulting in stroke, acute chest syndrome (chest pain, fever, dyspnea, and hypoxia), joint necrosis (especially head of femur and humerus), pain crises, acute and chronic organ dysfunction/failure, retinal hemorrhages, and increased risk of infection. Because SC erythrocytes sickle under ischemic conditions, they too are excellent candidates for use in the instant invention.

Erythrocytes with Hemoglobin S Antilles

Hemoglobin S Antilles show two mutations in hemoglobin S gene. The expected mutation of glutamic acid to valine at position β-6 similar to hemoglobin S is accompanied by a second substitution at position β-23 of valine to isoleucine. Since the mutation at β-23 produced no chance in the charge of the hemoglobin, it separated identically to hemoglobin S by standard techniques. Hemoglobin S Antilles is much less soluble than hemoglobin S. The consequence is that people heterozygous for hemoglobin A and hemoglobin S Antilles have symptoms and complications similar to those of patients with homozygous sickle cell disease. Because Hgb S Antilles erythrocytes sickle under hypoxic conditions, these cells are also excellent candidates for use in the present invention.

### Use of Sickle Erythroblasts and Erythrocytes In Vivo

### Subjects

The subjects treated are preferably human subjects and any mammalian species in which treatment or prevention of cancer is desirable, particularly agricultural and domestic mammalian species.

### Administration

Suitable methodology for administration of sickle erythrocytes, erythroblasts, sickle variants transduced with the various plasmids, vectors, oncolytic viruses, tumoricidal transgenes, proteins, antibodies, enzymes of the claimed invention is parenteral infusion or injection in a manner similar to a conventional blood transfusion with delivery between 5-1000 ml of cells/hr via a secure intravenous catheter.

### Dose

An effective dose of sickle erythrocytes is administered to a subject in need thereof. A “therapeutically effective amount” is an amount of the therapeutic composition sufficient to produce a measurable response (e.g., a cytolytic response in a subject being treated). Actual dosage levels of active ingredients in the pharmaceutical compositions of the claimed compositions are varied so as to administer an amount that is effective to achieve the desired therapeutic response for a particular subject.

The potency of a therapeutic composition can vary, and therefore a “therapeutically effective” amount can vary. However, using the assay methods described herein below, one skilled in the art can readily assess the potency and efficacy of a candidate modulator of this presently claimed subject matter and adjust the therapeutic regimen accordingly.

One of ordinary skill in the art can tailor the dosages to an individual patient, taking into account the particular formulation, method of administration to be used with the composition, and tumor size considering patient height and weight, severity and stage of symptoms, and the presence of additional deleterious physical conditions. Such adjustments or variations as well as evaluation of when and how to make such adjustments or variations are well known to those of ordinary skill.

Toxicity is assessed using criteria set forth by the National Cancer Institute and is reasonably defined as any grade 4 toxicity or any grade 3 toxicity persisting more than 1 week. Dose is also modified to maximize anti-tumor or anti-angiogenic activity.

### Tumors

The compositions of the claimed invention are useful in the treatment of both primary and metastatic solid tumors and carcinomas of the breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries, choriocarcinoma and gestational trophoblastic disease; male genital tract including prostate, seminal vesicles, testes and germ cell tumors; endocrine glands including thyroid, adrenal, and pituitary; skin including hemangiomas, melanomas, sarcomas arising from bone or soft tissues and Kaposi's sarcoma; tumors of the brain, nerves, eyes, and meninges including astrocytomas, gliomas, glioblastomas, retinoblastomas, neuromas, neuroblastomas, Schwannomas and meningiomas; solid tumors arising from hematopoietic malignancies such as leukemias and including chloromas, plasmacytomas, plaques and tumors of mycosis fungoides and cutaneous T-cell lymphoma/leukemia; lymphomas including both Hodgkin's and non-Hodgkin's lymphomas.

The compositions are also be useful for the prevention of metastases from the tumors described above either when used alone or in combination with radiotherapeutic, photodynamic, and/or chemotherapeutic treatments conventionally administered to patients for treating disorders, including angiogenic disorders. Treatment of a tumor with surgery, photodynamic therapy, radiation and/or chemotherapy is followed by administration of the compositions to extend the dormancy of micrometastases and to stabilize and inhibit the growth of any residual primary tumor or metastases. The compositions can be administered before, during, or after radiotherapy; before, during, or after chemotherapy; and/or before, during, or after photodynamic therapy.

### Pharmaceutical Compositions and Administration of SS Cells, SS Progenitor Cells or Genetically Modified SS Cells

The sickled erythrocytes may be administered parenterally preferably intravenously by infusion or injection but also may be implanted or injected intratumorally, intrapleurally, intrathecally, intrapericardially, intravesicularly, subcutaneously, intralymphatically, intraarticularly, intradermally, intracranially, intraarticularly or intramuscularly. They may be administered in a controlled release formulation.

The pharmaceutical compositions of the present invention will generally comprise an effective amount of sickled erythrocytes dissolved or dispersed in a pharmaceutically acceptable carrier or aqueous medium. One or more administrations may be employed, depending upon the lifetime of the drug at the tumor site and the response of the tumor to the drug. Administration may be by syringe, catheter or other convenient means allowing for introduction of a flowable composition. Administration may be every three days, weekly, or less frequent, such as biweekly or at monthly intervals.

The phrases “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by U.S. Food and Drug Administration. Supplementary active ingredients can also be incorporated into the compositions.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

### Injectable Formulations

The sickle cells compositions of the present invention are preferably formulated for parenteral administration, e.g., introduction by injection, infusion. They may also be administered intravenously, intramuscularly, intradermally, intraperitoneally, intrapleurally, intraarticularly. Means for preparing aqueous compositions that contain the SAg compositions are known to those of skill in the art in light of the present disclosure. Typically, such compositions can be prepared as for a typical blood transfusion, either as liquid solutions or suspensions; solid forms suitable for using to prepare solutions or suspensions upon the addition of a liquid prior to injection can also be prepared.

The techniques of preparation are generally well known in the art as exemplified by Remington's Pharmaceutical Sciences, 16th Ed. Mack Publishing Company, 1980, or most recent edition, incorporated herein by reference. Moreover, for human administration, preparations should meet sterility, pyrogenicity, general safety and purity standards as required by the U.S. Food and Drug Administration. Upon formulation, the therapeutic compositions are administered in a manner compatible with the dosage formulation and in such amount as is therapeutically effective.

### Animal Testing of Mature SS Cells, SS Progenitor Cells or Sickle Cells Carrying Tumoricidal Transgenes

The ability of murine mature SS cells, SS progenitor cells or iPPSCs carrying tumoricidal transgenes including siRNAs, shRNA, microRNAs and nucleic acids encoding oncolytic viruses/genomes, viruses carrying microRNAs and genes encoding dsRNA-caspase specific proteins described herein to induce a tumoricidal response after transfer to mice bearing various murine tumors is tested in murine tumor models given in the section titled “Tumor Models and Procedures for Evaluating Anti-Tumor Effects Studies.” In addition, several tumor types are implanted and their outgrowth assessed in several murine models of sickle cell disease shown in Table 3 (including mice with sickle-thalassemia containing little or no HbA hemoglobin). For this purpose, the Townes murine sickle cell model (UAB mouse) is especially useful. In addition, these SS models are used as sources of mature SS cells, SS progenitor cells (preferably carrying tumoricidal transgenes described above) for transfer to NOD SCID mice or irradiated syngeneic or allogeneic mice which after reconstitution may be implanted with syngeneic, allogeneic or xenogeneic tumors.

The Townes UAB SS model is useful for evaluating the outgrowth of subcutaneously implanted tumors in mice with C57B1/6 background. Such tumors include but are not limited to MCA 205-207 sarcoma, B16 melanoma, Lewis lung carcinoma, B16F10 melanoma, CT-26 colon carcinoma and hepatocellular carcinomas. Mature or progenitor SS cells obtained from the UAB mice are also transferred to NOD SCID mice or irradiated syngeneic or allogeneic mice after which syngeneic, allogeneic or xenogeneic tumors are implanted and their growth assesses. In the case of NOD SCID mice human SSiPPSCs and SSESCs (comprising tumoricidal transgenes) are also tested for their ability to kill xenogeneic (human tumors).

For cell transfer studies, mature SS cells from peripheral blood or SS progenitor cells transduced with tumoricidal transgenes are obtained from the bone marrow of SS mice. Methodology for preparation and administration of such murine and human SS progenitors, human SSiPPSCs and human SSEPCs are provided above. Such mice are rested for at least 8 weeks after SS progenitor SSiPPSCs and human SSEPCs transplantation to allow complete reconstitution after which tumor cells (10⁵-10⁶) are implanted subcutaneously and tumor growth assessed. Autologous and allogeneic transplantation with SSiPPSCs or SSEPCs for treatment or prevention of human tumors is described in Example 2.

TABLE 3
Transgenic Murine models for Sickle Cell disease

| Mouse | | | | | |
|---|---|---|---|---|---|
| Model | Mouse_Globin | Mouse_Globin | Human_Globin | Human_{—}^{s}Globin | %_{—}^{s} |
| Normal | Yes | Yes | No | No | 0 |
| New York | yes | Deleted | No | —^{s} | 32 |
| (NY) | | | | | |
| Berkeley | Yes | Deleted | No | —^{s}Antilies | 29 |
| (B) | | | | | |
| Hybrid(H) | Yes | Deleted | No | —^{s-Antilles} | 42.2 |
| [NY_B] | | | | —^{sAntilies} | 35.9 |
| Paszty(P) | Deleted | Deleted | Yes | —^{s} | 39 |

### Tumor Models and Procedures for Evaluating Anti-Tumor Effects Studies

The various sickle cell compositions described herein are tested for therapeutic efficacy in several well established rodent models which are considered to be highly representative of a broad spectrum of human tumors. These approaches are described in detail in Geran, R. I. et al., “Protocols for Screening Chemical Agents and Natural Products against Animal Tumors and Other Biological Systems (Third Edition)”, Canc. Chemother. Reports, Pt 3, 3:1-112, which is hereby incorporated by reference in its entirety.

In general, SS cells, SA cells, SS variant cells, SS porphyric cells, SS progenitors or SS ghosts loaded with tumoricidal virus, protein, drug, toxin, antibody, toxin-antibody conjugate optionally pre-treated with light therapy or photosensitizers as described herein or transfected with tumoricidal transgenes are tested in the animal models described in the next section.

Similarly these models are used to test the effectiveness of the SS progenitor cells as autologous or allogeneic transplants in mice with established tumors. These same cells can be used 1 or more days before tumor implantation to test their ability to prevent tumor outgrowth. The cells are administered to tumor bearing mice by intravenous infusion or injection in doses of 0.05 to 0.20 ml over 30 seconds to 2 minutes. The treatment is repeated every day or every second or third day for up to 10 treatments.

a. Calculation of Mean Survival Time (MST)
MST (days) is calculated according to the formula:

$$\frac{S + AS ( A - 1 ) - ( B + 1 ) NT}{S ( A - 1 ) - NT}$$

Day: Day on which deaths are no longer considered due to drug toxicity. For example, with treatment starting on Day 1 for survival systems (such as L1210, P388, B16, 3LL, and W256): Day A=Day 6; Day B=Day beyond which control group survivors are considered “no-takes.”
S: If there are “no-takes” in the treated group, S is the sum from Day A through Day B. If there are no “no-takes” in the treated group, S is the sum of daily survivors from Day A onward.
S(A−1): Number of survivors at the end of Day (A−1).
Example: for 3LE21, S(A−1)=number of survivors on Day 5.
NT: Number of “no-takes” according to the criteria given in Protocols 7.300 and 11.103.
B. T/C Computed for all treated groups

$$T \text{/} C = \frac{MST of treated group}{MST of control group} \times 100$$

Treated group animals surviving beyond Day Bare eliminated from calculations (as follows):

| No. of survivors in treated | Percent of “no-takes”in | |
|---|---|---|
| group beyond Day B | control group | Conclusion |
| 1 | Any percent | “no-take” |
| 2 | <10 | drug inhibition |
| | ³10 | “no-takes” |
| ³3 | <15 | drug inhibitions |
| | ³15 | “no-takes” |

Positive control compounds are not considered to have “no-takes” regardless of the number of “no-takes” in the control group. Thus, all survivors on Day B are used in the calculation of T/C for the positive control. Surviving animals are evaluated and recorded on the day of evaluation as “cures” or “no-takes.”

### Calculation of Median Survival Time (MedST)

MedST is the median day of death for a test or control group. If deaths are arranged in chronological order of occurrence (assigning to survivors, on the final day of observation, a “day of death” equal to that day), the median day of death is a day selected so that one half of the animals died earlier and the other half died later or survived. If the total number of animals is odd, the median day of death is the day that the middle animal in the chronological arrangement died. If the total number of animals is even, the median is the arithmetical mean of the two middle values. Median survival time is computed on the basis of the entire population and there are no deletion of early deaths or survivors, with the following exception:

C. Computation of MedST from Survivors

If the total number of animals including survivors (N) is even, the MedST (days) (X+Y)/2, where X is the earlier day when the number of survivors is N/2, and Y is the earliest day when the number of survivors (N/2)−1. If N is odd, the MedST (days) is X.

D. Computation of MedST from Mortality Distribution

If the total number of animals including survivors (N) is even, the MedST (days) (X+Y)/2, where X is the earliest day when the cumulative number of deaths is N/2, and Y is the earliest day when the cumulative number of deaths is (N/2)+1. If N is odd, the MedST (days) is X. “Cures” and “no-takes” in systems evaluated by MedST are based upon the day of evaluation. On the day of evaluation any survivor not considered a “no-take” is recorded as a “cure.” Survivors on day of evaluation are recorded as “cures” or “no-takes,” but not eliminated from the calculation.

E. Calculation of Approximate Tumor Weight from Measurement of Tumor Diameters with Vernier Calipers

The use of diameter measurements (with Vernier calipers) for estimating treatment effectiveness on local tumor size permits retention of the animals for lifespan observations. When the tumor is implanted sc, tumor weight is estimated from tumor diameter measurements as follows. The resultant local tumor is considered a prolate ellipsoid with one long axis and two short axes. The two short axes are assumed to be equal. The longest diameter (length) and the shortest diameter (width) are measured with Vernier calipers. Assuming specific gravity is approximately 1.0, and Pi is about 3, the mass (in mg) is calculated by multiplying the length of the tumor by the width squared and dividing the product by two. Thus,

$$Tumor weight ( mg ) = \frac{length ( mm ) \times ( width [ mm ] ) 2}{2} or \frac{L \times ( W ) 2}{2}$$

The reporting of tumor weights calculated in this way is acceptable inasmuch as the assumptions result in as much accuracy as the experimental method warrants.

### F. Calculation of Tumor Diameters

The effects of a drug on the local tumor diameter may be reported directly as tumor diameters without conversion to tumor weight. To assess tumor inhibition by comparing the tumor diameters of treated animals with the tumor diameters of control animals, the three diameters of a tumor are averaged (the long axis and the two short axes). A tumor diameter T/C of 75% or less indicates activity and a T/C of 75% is approximately equivalent to a tumor weight T/C of 42%.

G. Calculation of Mean Tumor Weight from Individual Excised Tumors

The mean tumor weight is defined as the sum of the weights of individual excised tumors divided by the number of tumors. This calculation is modified according to the rules listed below regarding “no-takes.” Small tumors weighing 39 mg or less in control mice or 99 mg or less in control rats, are regarded as “no-takes” and eliminated from the computations. In treated groups, such tumors are defined as “no-takes” or as true drug inhibitions according to the following rules:

| Percent of small tumors | Percent of “no-takes” | |
|---|---|---|
| in treated group | in control group | Action |
| ≦17 | Any percent | no-take; not used in |
| | | calculations |
| 18-39 | <10 | drug inhibition; use |
| | | in calculations |
| | ≧10 | no-takes; not used in |
| | | calculations |
| ≧40 | <15 | drug inhibition; use |
| | | in calculations |
| | ≧15 | Code all nontoxic |
| | | tests “33” |

Positive control compounds are not considered to have “no-takes” regardless of the number of “no-takes” in the control group. Thus, the tumor weights of all surviving animals are used in the calculation of T/C for the positive control (T/C defined above) SDs of the mean control tumor weight are computed the factors in a table designed to estimate SD using the estimating factor for SD given the range (difference between highest and lowest observation) (Biometrik Tables for Statisticians Pearson E S & Hartley H G eds. Cambridge Press, vol. 1, table 22, p. 165).

### II. Specific Tumor Models

### A. Melanotic Melanoma B16

Summary: Tumor cells (10⁵-10⁶) are implanted subcutaneously. Treatment begins 24 hours after either ip or sc implant or is delayed until an sc tumor of specified size (usually approximately 400 mg) can be palpated. Results expressed as a percentage of control survival time. The key parameter is mean survival time. Origin of tumor line: arose spontaneously in 1954 on the skin at the base of the ear in a C57BL/6 mouse (Handbook on Genetically Standardized Jax Mice. Jackson Memorial Laboratory, Bar Harbor, Me., 1962. See also Ann NY Acad Sci 100, Parts 1 and 2, (1963)).

| | |
|---|---|
| Animals | One sex used for all test and control animals in one |
| | experiment. |
| Propagation | C57BL/6 mice |
| Strain | |
| Tumor | Implant fragment sc by trochar or 12-g needle or tumor |
| Transfer | homogenate* every |
| | 10-14 days into axillary region with puncture in inguinal |
| | region. |
| Testing | C57BL/6 |
| Strain | |
| Time of | Excise sc tumor on Day 10-14 from donor mice and implant |
| Transfer | as above |
| Weight | Within a 3-g range, minimum weight of 18 g for males and |
| | 17 g for females. |
| Exp Size | 10/group; No. of control groups varies according to number |
| (n) | of test groups. |

*Tumor homogenate: Mix 1 g or tumor with 10 ml of cold balanced salt solution, homogenize, and implant 0.5 ml of tumor homogenate ip or sc. Fragment: A 25-mg fragment may be implanted sc.

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with therapeutic |
| | composition. Typically, mice receive 1 μg of the test composition |
| | in 0.5 ml saline. Controls receive saline alone. |
| | Treatment is one dose/week. Any surviving mice are sacrificed |
| | after 8 wks of therapy. |
| 5 | Weigh animals and record. |
| 60 | Kill all survivors and evaluate experiment. |

### Quality Control:

Acceptable control survival time is 14-22 days. Positive control compound is 5-fluorouracil: single dose is 200 mg/kg/injection, intermittent dose is 60 mg/kg/injection, and chronic dose is 20 mg/kg/injection. T/C lower limit for positive control compound is 135% Check control deaths, no takes, etc.

### Evaluation:

Compute mean animal weight on Days 1 and 5, and at the completion of testing compute T/C for all test groups with >65% survivors on Day 5. A T/C value of 85% indicates a toxic test. An initial T/C of 125% is considered necessary to demonstrate activity. A reproduced T/C 125% is considered worthy of further study. For confirmed activity a composition should have two multi-dose assays that produce a T/C 125%.

Metastasis after IV Injection of Tumor Cells

10⁵ B16 melanoma cells in 0.3 ml saline are injected intravenously in C57BL/6 mice. The mice are treated intravenously with 1 g of the composition being tested in 0.5 ml saline. Controls receive saline alone. Mice sacrificed after 4 weeks of therapy, the lungs are removed and metastases are enumerated.

### B. 3LL Lewis Lung Carcinoma

### Summary:

Tumor may be implanted sc as a 2-4 mm fragment, or im as a 2×10⁶-cell inoculum. Treatment begins 24 hours after implant or is delayed until a tumor of specified size (usually approximately 400 mg) can be palpated. Origin of tumor line: arose spontaneously in 1951 as carcinoma of the lung in a C57BL/6 mouse Cancer Res 15:39, (1955)). See also Malave I et al., J. Natl. Canc. Inst. 62:83-88 (1979).

| | |
|---|---|
| Animals | One sex used for all test and control animals in |
| | one experiment. |
| Propagation Strain | C57BL/6 mice |
| Tumor Transfer | Inject cells im in hind leg or implant fragment sc in |
| | axillary region with puncture in inguinal region. |
| | Transfer on day 12-14 |
| Testing Strain | C57BL/6 |
| Time of Transfer | Same as above |
| Weight | Within a 3-g range, minimum weight of 18 g for males |
| | and 17 g for females. |
| Exp Size (n) | 6/group for sc implant, or 10/group for im implant.; |
| | No. of control groups varies according to number |
| | of test groups. |

### Testing Schedule

| DAY | PROCEDURE |
|---|---|
| 0 | Implant tumor. Prepare materials. Run positive control in every |
| | odd-numbered experiment. Record survivors daily. |
| 1 | Weigh and randomize animals. Begin treatment with therapeutic |
| | composition. Typically, mice receive 1 μg of the test composition |
| | in 0.5 ml saline. Controls receive saline alone. Treatment is one |
| | dose/week. Any surviving mice are sacrificed after 4 wks of |
| | therapy. |
| 5 | Weigh animals and record. |
| Final day | Kill all survivors and evaluate experiment. |

### Quality Control:

Acceptable im tumor weight on Day 12 is 500-2500 mg. Acceptable im tumor MedST is 18-28 days. Positive control compound is cyclophosphamide: 20 mg/kg/injection, qd, Days 1-11. Check control deaths, no takes, etc.

### Evaluation:

Compute mean animal weight when appropriate, and at the completion of testing compute T/C for all test groups. When the parameter is tumor weight, a reproducible T/C of 42% is considered necessary to demonstrate activity. When the parameter is survival time, a reproducible T/C of 125% is considered necessary to demonstrate activity. For confirmed activity a composition must have two multi-dose assays

### C. 3LL Lewis Lung Carcinoma Metastasis Model

This model has been utilized by a number of investigators. See, for example, Gorelik, E. et al., J. Natl. Canc. Inst. 65:1257-1264 (1980); Gorelik, E. et al., Rec. Results Canc. Res. 75:20-28 (1980); Isakov, N. et al., Invasion Metas. 2:12-32 (1982) Talmadge J E et al., J. Nat'l. Canc. Inst. 69:975-980 (1982); Hilgard, P. et al., Br. J. Cancer 35:78-86(1977)).

Mice: male C57BL/6 mice, 2-3 months old. Tumor: The 3LL Lewis Lung Carcinoma was maintained by sc transfers in C57BL/6 mice. Following sc, im or intra-footpad transplantation, this tumor produces metastases, preferentially in the lungs. Single-cell suspensions are prepared from solid tumors by treating minced tumor tissue with a solution of 0.3% trypsin. Cells are washed 3 times with PBS (pH 7.4) and suspended in PBS. Viability of the 3LL cells prepared in this way is generally about 95-99% (by trypan blue dye exclusion). Viable tumor cells (3×10⁴-5×10⁶) suspended in 0.05 ml PBS are injected into the right hind foot pads of C57BL/6 mice. The day of tumor appearance and the diameters of established tumors are measured by caliper every two days. In experiments involving tumor excision, mice with tumors 8-10 mm in diameter are divided into two groups. In one group, legs with tumors are amputated after ligation above the knee joints. Mice in the second group are left intact as nonamputated tumor-bearing controls. Amputation of a tumor-free leg in a tumor-bearing mouse has no known effect on subsequent metastasis, ruling out possible effects of anesthesia, stress or surgery. Surgery is performed under Nembutal anesthesia (60 mg veterinary Nembutal per kg body weight).

### Determination of Metastasis Spread and Growth

Mice are killed 10-14 days after amputation. Lungs are removed and weighed. Lungs are fixed in Bouin's solution and the number of visible metastases is recorded. The diameters of the metastases are also measured using a binocular stereoscope equipped with a micrometer-containing ocular under 8× magnification. On the basis of the recorded diameters, it is possible to calculate the volume of each metastasis. To determine the total volume of metastases per lung, the mean number of visible metastases is multiplied by the mean volume of metastases. To further determine metastatic growth, it is possible to measure incorporation of ^{125I}dUrd into lung cells (Thakur M L et al., J. Lab. Clin. Med. 89:217-228 (1977)). Ten days following tumor amputation, 25 mg of ¹²⁵IdUrd is inoculated into the peritoneums of tumor-bearing (and, if used, tumor-resected mice. After 30 min, mice are given 1 mCi of ¹²⁵IdUrd. One day later, lungs and spleens are removed and weighed, and a degree of ¹²⁵IdUrd incorporation is measured using a gamma counter.

Statistics: Values representing the incidence of metastases and their growth in the lungs of tumor-bearing mice are not normally distributed. Therefore, non-parametric statistics such as the Mann-Whitney U-Test may be used for analysis.

### Example 1

For human studies, SS erythrocytes (SSRBCs) or SS nucleated erythrocyte precursors (SSEPCs) are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. The erythrocytes are a ABO- and Rh-matched for compatibility with recipients. Tumors of any type are susceptible to therapy with these agents. The cells are administered intravenously or intraarterially in a blood vessel perfusing a specific tumor site or organ, e.g. carotid artery, portal vein, femoral artery etc. over the same amount of time required for the infusion of a conventional blood transfusion. The quantity of cells to be administered in any one treatment ranges from one tenth to one half of a full unit of blood. The treatments are generally given every 2-7 days for a total of 1-12 treatments. However, the treatment schedule is flexible and may be given for a longer of shorter duration depending upon the patients' response.

All treated patients have histologically confirmed malignant disease including carcinomas, sarcomas, melanomas, lymphomas and leukemias and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. Histopathology is obtained to verify malignant disease.

Results: A total of 891 patients are patients treated. The number of patients for each tumor type and the results of treatment are summarized in Table 4. Positive tumor responses are observed in as high as 85-95% of the patients with breast, gastrointestinal, lung, prostate, renal and bladder tumors as well as melanoma and neuroblastoma as follows. Seven hundred and eighty three of 891 patients entered exhibit objective clinical responses for an overall response rate of 87.9%. Tumors generally start to diminish and objective remissions are evident after four weeks of therapy. Responses endure for an mean of 36 months.

TABLE 4

| | | | % of Patients |
|---|---|---|---|
| Patients/Tumors | No. | Response | Responding |
| All Patients | 891 | CR + PR | 87.9 |
| Breast adenocarcinoma | 165 | CR + PR | 91 |
| Gastrointestinal carcinoma | 156 | CR + PR | 90 |
| Lung Carcinoma | 200 | CR + PR | 95 |
| Brain glioma/astrocytoma | 60 | CR + PR | 85 |
| Prostate Carcinoma | 130 | CR + PR | 85 |
| Lymphoma/Leukemia | 61 | CR + PR | 80 |
| Head and Neck Cancer | 82 | CR + PR | 80 |
| Renal and Bladder Cancer | 53 | CR + PR | 95 |
| Melanoma | 67 | CR + PR | 85 |
| Neuroblastoma | 37 | CR + PR | 85 |

Toxicity consists of mild short-lived fever, fatigue and anorexia not requiring treatment. The incidence of side effects (as % of total treatments) are as follows: chills—12; fever—15; pain—6; nausea—3; respiratory—2; headache—2; tachycardia—4; vomiting—4; hypertension—1; hypotension—2; joint pain—3; rash—1; flushing—4; diarrhea—2; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed. Toxic effects usually associated with systemically administered chemotherapeutic agents are not observed.

### Example 2

For human studies, SS erythrocytes (SSRBCs) or nucleated SS erythrocyte precursors (SSEPCs) obtained from patients with homozygous S or sickle thalassemia hemoglobin, hemizygous sickle S and A hemoglobin, sickle hemoglobin-C disease, sickle beta plus thalassemia, The erythrocytes are ABO- and Rh-matched for compatibility with recipients are used. These erythrocytes express beta-2 adrenergic receptors operatively linked to granzyme and perforin. Neuroblastomas and pheochromocytomas are susceptible to therapy with these agents. The cells are administered intravenously or intraarterially in a blood vessel perfusing a specific tumor site or organ, e.g. carotid artery, portal vein, femoral artery etc. over the same amount of time required for the infusion of a conventional blood transfusion. The quantity of cells to be administered in any one treatment ranges from one tenth to one half of a full unit of blood. The treatments are generally given every 2-7 days for a total of 1-12 treatments. However, the treatment schedule is flexible and may be given for a longer of shorter duration depending upon the patients' response. All treated patients have histologically confirmed neuroblastoma or pheochromocytoma and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. Histopathology is obtained to verify malignant disease.

Results: A total of 1506 patients are patients treated. The overall number of patients for each tumor type and the results of treatment are summarized in Table 5. Positive tumor responses are observed in 83% of the patients with neuroblastoma and 81% of patients with pheochromocytoma as follows. Tumors generally start to diminish and objective remissions are evident after four weeks of therapy. Responses endure for a mean of 36 months.

TABLE 5
SS cells, SS progenitor cells transduced with beta-2
adrenergic receptor, granzyme and perforin

| | | | % of Patients |
|---|---|---|---|
| Patients/Tumors | No. | Response | Responding |
| All Patients | 1506 | CR + PR | 82 |
| Pheochromocytoma | 305 | CR + PR | 81 |
| Neuroblastoma | 1201 | CR + PR | 83 |

Toxicity consists of mild short-lived fever, fatigue and anorexia not requiring treatment. The incidence of side effects (as % of total treatments) are as follows: chills—12; fever—15; pain—6; nausea—3; respiratory—2; headache—2; tachycardia—4; vomiting—4; hypertension—1; hypotension —2; joint pain—3; rash—1; flushing—4; diarrhea—2; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed.

### Example 3

For human studies, SS erythrocytes (SSRBCs) or nucleated SS erythrocyte precursors (SSEPCs) are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia. The erythrocytes are ABO- and Rh-matched for compatibility with recipients. Mature or progenitor SS cell transfected with lentiviral or other suitable vector encoding a tumoricidal transgenes or TTORs as described herein.

Tumors of any type are susceptible to therapy with these agents. The cells are administered intravenously or intraarterially in a blood vessel perfusing a specific tumor site or organ, e.g. carotid artery, portal vein, femoral artery etc. over the same amount of time required for the infusion of a conventional blood transfusion. The quantity of cells to be administered in any one treatment ranges from one tenth to one half of a full unit of blood. The treatments are generally given every 2-7 days for a total of 1-12 treatments. However, the treatment schedule is flexible and may be given for a longer of shorter duration depending upon the patients' response. All treated patients have histologically confirmed malignant disease including carcinomas, sarcomas, melanomas, lymphomas and leukemias and have failed conventional therapy. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. Histopathology is obtained to verify malignant disease.

Results: A total of 1011 patients are patients treated with mature SS cells. All cells are stably transfected with or have encapsulated oncolytic virus as described herein and irradiated with light before intravenous administration. The overall number of patients for each tumor type and the results of treatment are summarized in Table 5. Positive tumor responses are observed in as high as 85-95% of the patients with breast, gastrointestinal, lung, prostate, renal and bladder tumors as well as melanoma and neuroblastoma as follows.

Eight hundred and ninty one of 1011 entered with all tumors exhibit objective clinical responses for an overall response rate of 88.9% (Table 6). Tumors generally start to diminish and objective remissions are evident after four weeks of therapy. Responses endure for a mean of 36 months.

TABLE 6
Mature SS cells or SS progenitor cells loaded with tumoricidal transgenes

| | | | % of Patients |
|---|---|---|---|
| Patients/Tumors | No. | Response | Responding |
| All Patients | 1011 | CR + PR | 88.7 |
| Breast adenocarcinoma | 165 | CR + PR | 90 |
| Gastrointestinal carcinoma | 156 | CR + PR | 90 |
| Lung carcinoma | 200 | CR + PR | 95 |
| Brain glioma/astrocytoma | 60 | CR + PR | 85 |
| Prostate carcinoma | 130 | CR + PR | 85 |
| Lymphoma/Leukemia | 61 | CR + PR | 80 |
| Head and Neck cancer | 82 | CR + PR | 80 |
| Renal and Bladder cancer | 53 | CR + PR | 95 |
| Melanoma | 67 | CR + PR | 85 |
| Neuroblastoma | 37 | CR + PR | 85 |

Toxicity consists of mild short-lived fever, fatigue and anorexia not requiring treatment. The incidence of side effects (as % of total treatments) are as follows: chills—12; fever—15; pain—6; nausea—3; respiratory—2; headache—2; tachycardia—4; vomiting—4; hypertension—1; hypotension—2; joint pain—3; rash—1; flushing—4; diarrhea—2; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed.

An additional 986 patients are treated with SS progenitor cells transduced with a tumoricidal transgene. The overall number of patients for each tumor type and the results of treatment are summarized in Table 7. Positive tumor responses are observed in 85-95% of patients with breast, gastrointestinal, lung, prostate, renal and bladder tumors as well as melanoma and neuroblastoma. Eighty nine percent of 986 patients entered with all tumors exhibit objective clinical responses. Tumors generally start to diminish and objective remissions are evident after three to four weeks of therapy. Responses endure for a mean of 36 months.

TABLE 7
Mature SS cells or SS progenitor cells loaded with anti-tumor drugs

| | | | % of Patients |
|---|---|---|---|
| Patients/Tumors | No. | Response | Responding |
| All Patients | 986 | CR + PR | 89.0 |
| Breast adenocarcinoma | 162 | CR + PR | 90 |
| Gastrointestinal carcinoma | 153 | CR + PR | 90 |
| Lung carcinoma | 195 | CR + PR | 95 |
| Brain glioma/astrocytoma | 57 | CR + PR | 85 |
| Prostate carcinoma | 127 | CR + PR | 85 |
| Lymphoma/Leukemia | 61 | CR + PR | 80 |
| Head and Neck Cancer | 80 | CR + PR | 80 |
| Renal and Bladder cancer | 51 | CR + PR | 95 |
| Melanoma | 63 | CR + PR | 85 |
| Neuroblastoma | 37 | CR + PR | 85 |

Toxicity consists of mild fatigue, anorexia and nausea not requiring treatment. The incidence of side effects (as % of total treatments) are as follows: fatigue—15; nausea—12; anorexia—10; chills—3; fever—1; pain—2; respiratory—2; headache—2; tachycardia—4; vomiting—4; hypertension—2; hypotension—1; joint pain—2; rash—1; flushing—1; diarrhea—4; itching/hives—1; bloody nose—1; dizziness—<1; cramps—<1; feeling faint—<1; twitching—<1; blurred vision—<1; gastritis<1; redness on hand—<1.

### Example 4

Autologous or allogeneic CD34+ stem cells containing SCA+ erythroid progenitors from normal donors transduced with SS beta globin genes using gene editing technology as described herein are used for transplantation. An additional source of these CD34+ stem cells or erythroid progenitors is from patients with sickle cell anemia or its variants as described in Examples 1-3. These cells are obtained directly from the bone marrow or peripheral blood of the recipient or related or unrelated allogeneic donor after short induction period. Bone marrow is obtained by repeated aspiration of the posterior iliac crests while the donor is under general or local anesthesia. Peripheral blood is a convenient source of these hematopoietic stem cells and has replaced marrow for autologous and most allogeneic transplantations. Induction treatment consists of 3 to 6 cycles of chemotherapy or other anticancer treatment in order to reduce the tumor burden. Stem-cell mobilization from the bone marrow is effected using granulocyte colony-stimulating factor (G-CSF), either alone or with cyclophosphamide and optionally AMD3100—a small-molecule reversible inhibitor of CXC chemokine receptor 4 (CXCR4). Peripheral blood stem cell are obtained via leukapheresis wherein up to 25 liters of blood is processed in four hours yielding enough CD34+ peripheral-blood stem cells to ensure rapid engraftment. After collection, stem cells are usually cryopreserved in dimethyl sulfoxide at temperatures below −120° C. The cells are transfected with nucleic acids encoding SS hemoglobin as described above and expanded in vitro to obtain sufficient numbers for transplantation. The minimal dose of CD34+ cells necessary for safe engraftment is 2×10⁶/kilogram of body weight. The hematopoietic stem cells are infused through a central venous catheter at a rate ranging from 5 to 20 ml per minute. The patient may be premedicated with an antihistamine, an antiemetic agent, an antipyretic agent, and corticosteroids to mitigate infusion reactions. The infusion is performed in a reverse-isolation room. The infusion of autologous stem cells itself is associated with a variety of adverse effects, including nausea and vomiting, headache, and chills and fever. In most patients, such effects are mild and transient. Blood counts are monitored weekly.

Autologous transplantation is followed by nonmyeloablative allogeneic transplantation. This strategy combines the tumor cytoreduction of a high-dose autologous transplant with the lowered treatment-related mortality (TRM) of a nonmyeloablative conditioning regimen (described below).

For allogeneic SS cell progenitor transfer ABO and DNA typing of donor and recipient is carried out to obtain closely matched donors. The use of a closely matched donor increases the chances of successful engraftment and reduces the risk of GVHD. Donors undergo 5 to 6 days of granulocyte colony stimulating factor (G-CSF) mobilization (10 to 16 μg/kilogram of body weight/day), followed by large-volume leukapheresis, with the goal of collecting at least 10×10⁶ CD34+ cells per kilogram of the recipient's body weight; the donor's cells are cryopreserved. Donor cells are further transfected with SS genes in a fashion identical to that for autologous transplantation. The conditioning regimen for the recipient consists of Alemtuzumab, a humanized monoclonal antibody directed against CD52 (expressed on lymphocytes), depletes T cells and B cells. It does not affect the development of hematopoietic stem cells and has been used to prevent GVHD at a total dose of 1 mg/kg given over a period of 5 days in gradually increasing doses: 0.03 mg per kilogram (test dose) on day-7, 0.1 mg per kilogram on day-6, and then 0.3 mg per kilogram per day on days-5 through-3. A single dose of 300 cGy of total-body irradiation (TBI) was administered on day-2 (with gonadal shielding applied for men). Treatment with oral sirolimus is initiated on day-1 at a dose of 5 mg every 4 hours for three doses, then 5 mg daily starting on day 0, modified to achieve target trough levels of 10 to 15 ng per milliliter of whole blood. Tapering of sirolimus can be initiated when donor chimerism reached 100%

Additional non-myeloablative regimen to prevent graft rejection and increase pretransplantation host T-cell immunosuppression consists of the administration of fludarabine at 30 mg/m2 was added to the conditioning regimen of 2 Gy TBI. Postgrafting immunosuppression consisted of cyclosporine or tacrolimus combined with mycophenolate. This nonmyeloablative conditioning regimen is associated with shorter in-patient hospital stays, reduced need for transfusions and a shorter duration of neutropenia with fewer bacterial infections. Postgrafting immunosuppression consisted of cyclosporine or tacrolimus.

A variety of reduced-intensity conditioning regimens have been studied. Most of these regimens are based on fludarabine and an alkylating agent (e.g., melphalan, cyclophosphamide or busulfan), with or without the addition of anti-T-lymphocyte antibodies, such as ATG or the anti-CD52 antibody, alemtuzumab. The spectrum of ablative and non-ablative regimens useful in this invention are given in Gyurkicza B Expert Opin Hematol. 3: 285-299 (2910) incorporated by reference with its references in entirety.

If transplantation is urgent or if suitable donors are not found, cord blood can be used. Blood from the umbilical cord and the placenta is rich in hematopoietic stem cells but limited in volume. It is collected immediately after birth and then frozen. The transplantation of cord blood requires less-stringent HLA matching than the transplantation of adult peripheral blood or marrow, because mismatched cord-blood cells are less likely to cause GVHD. The results are better with fewer HLA mismatches and greater numbers of CD34+ cells. Cord-blood stem cells can be transfected and expanded in vitro as described above for autotransplantation. The less-stringent HLA requirements for cord-blood transplantation would permit a smaller donor pool to serve virtually all potential recipients.

The SS gene transduced progenitor cells are administered intravenously or intraarterially in a blood vessel perfusing a specific tumor site or organ, e.g. carotid artery, portal vein, femoral artery etc. over the same amount of time required for the infusion of a conventional blood transfusion. The quantity of cells to be administered in any one treatment ranges from one tenth to one half of a full unit of blood. The treatments are generally given every 2-7 days for a total of 1-12 treatments. However, the treatment schedule is flexible and may be given for a longer of shorter duration depending upon the patients' response. Post transplantation, the SS cell hematocrit is adjusted with weekly erythropoietin dosing to range between 5 and 10% of the total hemoglobin.

All treated patients have histologically confirmed malignant disease including carcinomas, sarcomas, melanomas, lymphomas and leukemias and have failed conventional therapy. Tumors of any type are susceptible to therapy with SS stem cell transplants. Patients may be diagnosed as having any stage of metastatic disease involving any organ system. Staging describes both tumor and host, including organ of origin of the tumor, histologic type and histologic grade, extent of tumor size, site of metastases and functional status of the patient. A general classification includes the known ranges of Stage I (localized disease) to Stage 4 (widespread metastases). Patient history is obtained and physical examination performed along with conventional tests of cardiovascular and pulmonary function and appropriate radiologic procedures. Histopathology is obtained to verify malignant disease.

Results: A total of 1239 patients receiving autologous SS cell transplant and 1223 patients received allogeneic SS cell transplant. The overall number of patients for each tumor type and the results of treatment are summarized in Tables 8 and 9. Positive tumor responses are observed in 85-95% of the patients with breast, gastrointestinal, lung, prostate, renal and bladder tumors as well as melanoma and neuroblastoma as follows. Tumors generally start to diminish and objective remissions are evident after four weeks of therapy. Responses endure for a mean of 36 months.

TABLE 8
Autologous SS progenitor cell transplant

| | | | % of Patients |
|---|---|---|---|
| Patients/Tumors | No. | Response | Responding |
| All Patients | 1239 | CR + PR | 88.3 |
| Breast adenocarcinoma | 201 | CR + PR | 92 |
| Gastrointestinal carcinoma | 192 | CR + PR | 91 |
| Lung carcinoma | 241 | CR + PR | 90 |
| Brain glioma/astrocytoma | 78 | CR + PR | 86 |
| Prostate carcinoma | 151 | CR + PR | 87 |
| Lymphoma/Leukemia | 76 | CR + PR | 89 |
| Head and Neck cancer | 91 | CR + PR | 83 |
| Renal and Bladder cancer | 61 | CR + PR | 96 |
| Melanoma | 94 | CR + PR | 83 |
| Neuroblastoma | 54 | CR + PR | 88 |

Chimerism is achieved in 87% of these patient vs 80% with allogeneic SS progenitor cell transplants. However, autologous transplant recipients showed no graft versus host reactions, mucositis, sinus obstructive symptoms, opportunistic infections or neutropenia compared to the recipients of allogeneic cell transplants (Table 8).

TABLE 9
Complications of SS Progenitor Cell Transplantation

| | Autologous | Allogeneic |
|---|---|---|
| | SSPT | SSPT |
| Chimerism achieved | 87 | 80 |
| Hemoglobin S levels | 38% | 35% |
| Acute GVHD | none | 75% |
| Mucositis | none | 30% |
| Sinusoidal Obstructive | none | 12% |
| Syndrome | | |
| Opportunistic | none | 15% |
| Infections | | |
| Neutropenia | 0 | 85% |

Toxicity consists of mild short-lived fever, fatigue and anorexia not requiring treatment. The incidence of side effects (as % of total treatments) are as follows: chills—12; fever—70; pain—6; nausea—3; respiratory—2; headache—2; tachycardia—4; vomiting—4; hypertension—1; hypotension—2; joint pain—3; rash—1; flushing—4; diarrhea—2; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed.

In an additional study, 1223 patients are treated with allogeneic SS progenitor cell transplants The overall number of patients for each tumor type and the results of treatment are summarized in Table 10. Positive tumor responses are observed in 84-92% of the patients with breast, gastrointestinal, lung, prostate, renal and bladder tumors as well as melanoma and neuroblastoma. Eighty eight percent of all patients with tumors exhibit objective clinical responses. Tumors generally start to diminish and objective remissions are evident after three to four weeks of therapy. Responses endure for a mean of 36 months.

TABLE 10
Allogeneic SS progenitor cells transplant

| | | | % of Patients |
|---|---|---|---|
| Patients/Tumors | No. | Response | Responding |
| All Patients | 1223 | CR + PR | 88.5 |
| Breast adenocarcinoma | 197 | CR + PR | 91 |
| Gastrointestinal carcinoma | 173 | CR + PR | 94 |
| Lung carcinoma | 241 | CR + PR | 92 |
| Brain glioma/astrocytoma | 78 | CR + PR | 86 |
| Prostate carcinoma | 172 | CR + PR | 85 |
| Lymphoma/Leukemia | 81 | CR + PR | 81 |
| Head and Neck cancer | 80 | CR + PR | 87 |
| Renal and Bladder cancer | 68 | CR + PR | 96 |
| Melanoma | 79 | CR + PR | 89 |
| Neuroblastoma | 54 | CR + PR | 84 |

Toxicity consists of mild fatigue, anorexia and nausea not requiring treatment. The incidence of side effects (as % of total treatments) are as follows: fatigue—15; nausea—12; anorexia—10; chills—41; fever—97; pain—2; respiratory—2; headache—2; tachycardia—4; vomiting—4; hypertension—2; hypotension—1; joint pain—67; rash—1021; flushing—1; diarrhea—101; itching/hives—1; bloody nose—25; dizziness—<1; cramps—<1; feeling faint—<1; twitching—<1; blurred vision—<1; gastritis<1; redness on hand—<1.

### Example 5

For human studies, SSiPPSCs or SSESCs are obtained from patients with homozygous S or sickle thalassemia hemoglobin, hemizygous sickle S and A hemoglobin, sickle hemoglobin-C disease, sickle beta plus thalassemia, sickle hemoglobin-D disease, sickle hemoglobin-E disease, homozygous C or C-thalassemia, hemoglobin-C beta plus thalassemia, homozygous E or E-thalassemia from iPPSCs or embryonic SS stem cells. SS erythroid progenitors and mature SSRBCs are isolated and expanded as described herein. The mature erythrocytes from both sources are preferentially O+ and negative for Kidd, Kell, C, D, E antigens and Rh⁻ for compatibility with recipients. The erythroid progenitors are HLA allotyped to provide an acceptably close match with the recipients. Erythroid progenitor SS cells from either source are transduced with lentiviral or other suitable vector encoding a tumoricidal transgenes or oncogenic virus or genome or regulatory RNAs as described herein. This trial consists of an equal number of patients divided into four cohorts as follows: i) Mature SSRBCs from SSiPPSCs. ii) Mature SSRBCs from SS erythroid embryonic stem cells. iii) SS progenitor cell from SSiPPSCs. iv) SS progenitor cells from SS erythroid embryonic stem cells.

The SS cells are preferably administered intravenously. They may also be delivered intraarterially in a blood vessel perfusing a specific tumor site or organ, e.g. carotid artery, portal vein, femoral artery etc. over the same amount of time required for the infusion of a conventional blood transfusion. The quantity of cells to be administered in any one treatment ranges from one tenth to one unit of packed cells. The treatments are generally given every 2-7 days for a total of 1-12 treatments. However, the treatment schedule is flexible and may be given for a longer of shorter duration depending upon the patients' response. Tumors of any type are susceptible to therapy with these agents. All treated patients have histologically confirmed malignant disease including carcinomas, sarcomas, melanomas, lymphomas and leukemias and have failed conventional therapy. Patients may be diagnosed as having any stage (1-4) of malignant disease including metastases in any organ system. Karnofsky performance should be 60 or greater. Patient history is obtained and physical examination performed along with conventional tests of cardiovascular and pulmonary function and appropriate radiologic procedures. Histopathology is obtained to verify malignant disease.

Results: For each of the four cohorts, the number of patients treated for each tumor type and the results of treatment are summarized in Tables 11-14. Positive tumor responses are observed in 85-95% of the patients with breast, gastrointestinal, lung, prostate, renal and bladder tumors as well as melanoma and neuroblastoma as follows. Tumors generally start to diminish and objective remissions are evident after four weeks of therapy. Responses endure for a mean of 36 months.

TABLE 11
Mature SSRBCs derived from SS iPPSCs loaded
with tumoricidal transgenes

| | | | % of Patients |
|---|---|---|---|
| Patients/Tumors | No. | Response | Responding |
| All Patients | 1011 | CR + PR | 83.0 |
| Breast adenocarcinoma | 165 | CR + PR | 82 |
| Gastrointestinal carcinoma | 156 | CR + PR | 90 |
| Lung carcinoma | 200 | CR + PR | 83 |
| Brain glioma/astrocytoma | 60 | CR + PR | 82 |
| Prostate carcinoma | 130 | CR + PR | 80 |
| Lymphoma/Leukemia | 61 | CR + PR | 90 |
| Head and Neck cancer | 82 | CR + PR | 80 |
| Renal and Bladder cancer | 53 | CR + PR | 81 |
| Melanoma | 67 | CR + PR | 86 |
| Neuroblastoma | 37 | CR + PR | 84 |

TABLE 12
Mature SSRBCs derived from erythroid embryonic stem
cells loaded with tumoricidal transgenes

| | | | % of Patients |
|---|---|---|---|
| Patients/Tumors | No. | Response | Responding |
| All Patients | 1024 | CR + PR | 85.5 |
| Breast adenocarcinoma | 180 | CR + PR | 84 |
| Gastrointestinal carcinoma | 166 | CR + PR | 81 |
| Lung carcinoma | 220 | CR + PR | 91 |
| Brain glioma/astrocytoma | 80 | CR + PR | 82 |
| Prostate carcinoma | 150 | CR + PR | 81 |
| Lymphoma/Leukemia | 81 | CR + PR | 89 |
| Head and Neck cancer | 10 | CR + PR | 80 |
| Renal and Bladder cancer | 73 | CR + PR | 94 |
| Melanoma | 77 | CR + PR | 90 |
| Neuroblastoma | 57 | CR + PR | 83 |

TABLE 13
SS erythroid progenitor cells derived from SS iPPSCs
loaded with tumoricidal transgenes

| | | | % of Patients |
|---|---|---|---|
| Patients/Tumors | No. | Response | Responding |
| All Patients | 1116 | CR + PR | 87.4 |
| Breast adenocarcinoma | 175 | CR + PR | 91 |
| Gastrointestinal carcinoma | 166 | CR + PR | 81 |
| Lung carcinoma | 210 | CR + PR | 93 |
| Brain glioma/astrocytoma | 70 | CR + PR | 87 |
| Prostate carcinoma | 140 | CR + PR | 89 |
| Lymphoma/Leukemia | 71 | CR + PR | 86 |
| Head and Neck cancer | 92 | CR + PR | 84 |
| Renal and Bladder cancer | 63 | CR + PR | 82 |
| Melanoma | 77 | CR + PR | 94 |
| Neuroblastoma | 52 | CR + PR | 87 |

TABLE 14
SS erythroid progenitor cells derived from SS embryonic
stem cells loaded with tumoricidal transgenes

| | | | % of Patients |
|---|---|---|---|
| Patients/Tumors | No. | Response | Responding |
| All Patients | 1021 | CR + PR | 86.4 |
| Breast adenocarcinoma | 181 | CR + PR | 90 |
| Gastrointestinal carcinoma | 167 | CR + PR | 82 |
| Lung carcinoma | 219 | CR + PR | 92 |
| Brain glioma/astrocytoma | 79 | CR + PR | 85 |
| Prostate carcinoma | 146 | CR + PR | 81 |
| Lymphoma/Leukemia | 73 | CR + PR | 87 |
| Head and Neck cancer | 84 | CR + PR | 84 |
| Renal and Bladder cancer | 51 | CR + PR | 88 |
| Melanoma | 69 | CR + PR | 93 |
| Neuroblastoma | 52 | CR + PR | 82 |

Toxicity consists of mild short-lived fever, fatigue and anorexia not requiring treatment. The incidence of side effects (as % of total treatments) are as follows: chills—12; fever—15; pain—6; nausea—3; respiratory—2; headache—2; tachycardia—4; vomiting—4; hypertension—1; hypotension—2; joint pain—3; rash—1; flushing—4; diarrhea—2; itching/hives—1; bloody nose—1; dizziness—<1; cramps—<1; fatigue—<1; feeling faint—<1; twitching—<1; blurred vision—<1; gastritis<1; redness on hand—<1. Fever and chills are the most common side effects observed.

All the references patents and patent application cited above in this patent application including those below and their references are incorporated by reference in entirety. Whether specifically incorporated or not. In addition, the following patent applications and their references are incorporated by reference in entirety with their references.

| Inventor | Ser. No. | Filing Date | Title |
|---|---|---|---|
| Terman, D. S. | 13/328,748 | Dec. 16, 2011 | Compositions and Methods for Treatment of Cancer |
| Terman, D. S. | 14,037,176 | Sep. 25, 2013 | Compositions and Methods for Treatment of Cancer |
| Terman, D. S. | 61,807,457 | Apr. 2, 2013 | Sickled Erythrocytes Alone or with Anti-tumor Agents Induce Tumor |
| | | | Vaso-occlusion and Tumoricidal Effects |
| Terman, D. S. | 13/367,797 | Feb. 7, 2012 | Sickled Erythrocytes with Anti-tumor Agents Induce Tumor Vaso- |
| | | | occlusion and Tumoricidal Effects |
| Terman, D. S. | 13/317,590 | Oct. 20, 2011 | Compositions and Methods for Treatment of Cancer |
| Terman, D. S. | 61/455,592 | Oct. 20, 2010 | Compositions and Methods for Treatment of Cancer |
| Terman, D. S. | 12/586,532 | Sep. 22, 2009 | Sickled Erythrocytes with Anti-tumor Agents Induce Tumor |
| | | | Vaso-occlusion and Tumoricidal Effects |
| Terman D. S. | 12/276,941 | Nov. 24, 2008 | Compositions and Methods for Treatment of Cancer |
| Terman D. S. | 12/145,949 | Jun. 25, 2008 | Compositions and Methods for Treatment of Cancer |
| Terman D. S. | 10/937,758 | Sep. 8, 2004 | Compositions and Methods for Treatment of Cancer |
| Terman, D. S. | 61,215,906 | May 11, 2009 | Sickled Erythrocytes, Nucleated Precursors & Erythroleukemia Cells |
| | | | for Targeted Delivery of Tumoricidal Agents |
| Terman, D. S | 61/211,227 | Mar. 28, 2009 | Sickled Erythrocytes, Nucleated Precursors & Erythroleukemia Cells |
| | | | for Targeted Delivery of Tumoricidal Agents |
| Terman, D. S. | 61/206,338 | Jan. 28, 2009 | Sickled Erythrocytes, Nucleated Precursors & Erythroleukemia Cells |
| | | | for Targeted Delivery of Tumoricidal Agents |
| Terman D. S. | 61/205,776 | Jan. 22, 2009 | Sickled Erythrocytes Induced Tumor Vaso-occlusion and |
| | | | Tumoricidal Effects |
| Terman, D. S. | 61/192,949 | Sep. 22, 2008 | Sickled Erythrocytes, Nucleated Precursors & Erythroleukemia Cells |
| | | | for Targeted Delivery of Oncolytic Viruses, Anti-tumor Proteins, |
| | | | Plasmids, Toxins, Hemolysins and Chemotherapy |
| Terman, D. S. | PCT/US07/6986 | May 29, 2007 | Sickled Erythrocytes, Nucleated Precursors & Erythroleukemia Cells |
| Dewhirst M. W. | 9 | | for Targeted Delivery of Oncolytic Viruses, Anti-tumor Proteins, |
| | | | Plasmids, Toxins, Hemolysins and Chemotherapy |
| Terman, D. S. | 60/842,213 | Sep. 5, 2006 | Sickled Erythrocytes & Nucleated Precursors for Targeted Delivery |
| | | | of Oncolytic Toxins, Viruses, hemolysins and chemotherapy |
| Terman, D. S. | 60/819,551 | Jul. 8, 2006 | Sickled Erythrocytes & Nucleated Precursors for Targeted Delivery |
| | | | of Oncolytic Toxins, Viruses, hemolysins and chemotherapy |
| Terman, D. S. | 60/809,553 | May 30, 2006 | Sickled Erythrocytes & Nucleated Precursors for Targeted Delivery |
| | | | of Oncolytic Toxins, Viruses, hemolysins and chemotherapy |
| Terman, D. S. | 60/799514 | May 10, 2006 | Synergy of Superantigens, Cytokines and Chemotherapy in |
| Bohach, G | | | Treatment of Malignant Disease |
| Terman, D. S, Etiene, | PCTUS05/02263 | Jun. 27, 2005 | Enterotoxin Gene Cluster Superantigens (egc) to Treat Malignant |
| J., Vandenesch, F., | 8 | | Disease |
| Lina, G. Bohach, G. | | | |
| Terman, D. S, Etiene, | 60/583,692 | Jun. 29, 2004 | Enterotoxin Gene Cluster Superantigens (egc) to Treat Malignant |
| J., Vandenesch, F., | | | Disease |
| Lina, G. Bohach, G. | | | |
| Terman, D. S. | 60/665, 654 | Mar. 23, 2005 | Enterotoxin Gene Cluster Superantigens (egc) to Treat Malignant |
| | | | Disease |
| Terman, D. S, Etiene, | 60/626,159 | Nov. 6, 2004 | Enterotoxin Gene Cluster Superantigens (egc) to Treat Malignant |
| J., Vandenesch, F., | | | Disease |
| Lina, G. Bohach, G. | | | |
| Terman, D. S. | 60/583,692 | Jun. 29, 2004 | Intrathecal and Intrapleural Superantigens to Treat Malignant |
| | | | Disease |
| Terman, D. S. | 60/550,926 | Mar. 5, 2004 | Intrathecal and Intrapleural Superantigens to Treat Malignant |
| | | | Disease |
| Terman, D. S. | 60/539,863 | Jan. 27, 2004 | Intrathecal and Intrapleural Superantigens to Treat Malignant |
| | | | Disease |
| Terman, D. S. | PCT/US03/1438 | May 8, 2003 | Intrathecal and Intrapleural Superantigens to Treat Malignant |
| | 1 | | Disease |
| Terman, D. S. | 10/428,817 | May 5, 2003 | Composition and Methods for Treatment of Neoplastic Diseases |
| Terman, D. S. | 60/438,686 | Jan. 9, 2003 | Intrathecal and Intrapleural Superantigens to Treat Malignant |
| | | | Disease |
| Terman, D. S. | 60/415,310 | Oct. 1, 2002 | Intrathecal and Intratumoral Superantigens to Treat Malignant |
| | | | Disease. |
| Terman, D. S. | 60/406,750 | Aug. 29, 2002 | Intrathecal Superantigens to Treat Malignant Fluid Accumulation |
| Terman, D. S. | 60/415,400 | Oct. 2, 2002 | Composition and Methods for Treatment of Neoplastic Diseases |
| Terman, D. S. | 60/406,697 | Aug. 28, 2002 | Compositions and Methods for Treatment of Neoplastic Diseases |
| Terman, D. S. | 60/389,366 | Jun. 15, 2002 | Compositions and Methods for Treatment of Neoplastic Diseases |
| Terman, D. S. | 60/378,988 | May 8, 2002 | Compositions and Methods for Treatment of Neoplastic Diseases |
| Terman, D. S. | 09/870,759 | May 30, 2001 | Compositions and Methods for Treatment of Neoplastic Diseases |
| Terman, D. S. | 09/751,708 | Dec. 28, 2000 | Compositions and Methods for Treatment of Neoplastic Diseases |
| Terman, D. S. | 09/640,884 | Aug. 30, 2000 | Compositions and Methods for Treatment of Neoplastic Diseases |
| Terman, D. S. | 60/151,470 | Aug. 30, 1999 | Compositions and Methods for Treatment of Neoplastic Diseases |

Having now fully described this invention, it will be appreciated by those skilled in the art that the same can be performed within a wide range of equivalent parameters, concentrations, and conditions without departing from the spirit and scope of the invention and without undue experimentation.

We claim:

1. A method of treating a subject with a tumor comprising the administration of an effective amount of mature or progenitor erythrocytes or pluripotent erythroid stem cells containing at least one hemoglobin S allele comprising
(a) introducing into said erythroid progenitor cells or said pluripotent erythroid stem cells a recombinant nucleic acid vector comprising a gene(s) encoding one or a plurality of tumoricidal transgenes encoding tumoricidal proteins or nucleotides, an erythroid specific β-globin promoter/enhancer, the 2^{nd} β-globin intron and poly A sequence, the β-globin locus control region comprising at least one erythroid specific β-globin DNase I hypersensitive site, and
(b) allowing said erythroid progenitor cells or said said pluripotent erythroid stem cells to express said tumoricidal transgene encoding said tumoricidal protein or nucleotide in said sickle erythroid progenitor cell or said pluripotent erythroid stem cells, or
(c) allowing said erythroid progenitor cells or said pluripotent erythroid stem cells to differentiate into mature sickle erythrocytes wherein said tumoricidal transgene encoding said tumoricical protein or nucleotide are expressed in said mature erythrocytes, and
(d) administering said sickle erythroid progenitor cells or said pluripotent erythroid stem cells or said mature sickle erythrocytes expressing said tumoricidal transgenes encoding said tumoricidal proteins or nucleotides to a mammal with cancer, and
(e) inducing a tumoricidal response.

2. The method according to claim 1, wherein said tumoricidal transgene encoding tumoricidal proteins or nucleotides loaded into said recombinant nucleic acid vector is an anti-tumor virus or viral genome, a toxin, an siRNA, an shRNA, a microRNA, chemokine, antitumor cytokine.

3. The method according to claim 1, wherein said mature or progenitor erythrocytes or pluripotent erythroid stem cells containing at least one hemoglobin S allele are selected from a group consisting of erythrocytes containing SS hemoglobin, erythrocytes containing SA hemoglobin, erythrocytes containing SC hemoglobin, erythrocytes containing SD hemoglobin, erythrocytes containing SE hemoglobin, erythrocytes containing Antilles hemoglobin and erythrocytes containing S beta plus thalassemia hemoglobin.

4. The method according to claim 1 wherein said recombinant nucleic acid vector is of lentiviral origin.

5. The method according to claim 1 wherein said tumoricidal transgene or nucleotide is linked to a cell penetrating nucleotide.

6. The method according to claim 2 wherein said antitumor virus or viral genome is selected from the group consisting of herpes simplex, adenovirus, vaccinia, Newcastle Disease virus, reovirus and autonomous parvovirus, vesicular stomatitis virus, Sindbis virus.

7. The method according to claim 2 wherein said toxin is selected from selected from a group comprising, a perforin, a granzymes, a granulysin, a pseudomonas exotoxin, a pseudomonas homologues and fusion proteins, a pertussis toxin, a Shiga toxin, a diptheria toxin, a diptheria homologue or fusion protein, ricin toxin, a granzyme B, a perforin, a complement membrane attack complex,

8. The method according to claim 2 wherein the siRNA, shRNA, microRNA or viruses comprising microRNA selected from a group targeting mRNAs encoding heme oxygenase, nitrous oxide synthase, HIF1-1α, p53, RAS, CXCR4, p-Catenin, bcl-2, PLK-1, Somatostatin, Raf-1, c-raf, EGFR, HER-2, VEGL, HIF1-1α, Skp-2. MMP-9+, Cathepsin, PLK1, VEGF-R2, EWS-FLI1, Rad51, c-myc MDM2, VEGF, FGF-4, EZH2, p110α.

9. The method according to claim 2 wherein the said miRNA is let-7, miRNA 17-92, miRNA 155, miRNA 93

10. The method according to claim 2 wherein said antitumor cytokine is IL-12, TNFα, INFγ, IFNα, IFNβ, complement membrane attack complex,

11. The method according to claim 2, wherein said toxin consists of:
(i) a wild type staphylococcal enterotoxin or wild type streptococcal pyrogenic exotoxin protein which wild type protein has the biological activity of stimulating T cell mitogenesis via a T cell receptor vβ region;
(ii) a biologically active variant or fragment of a wild type staphylococcal enterotoxin or streptococcal pyrogenic exotoxin, which variant or fragment:
(a) has the biological activity of stimulating T cell mitogenesis via a T cell receptor vβ region and
(b) has sequence homology characterized as a z value exceeding 13 when the sequence of the variant or fragment is compared to the sequence of a wild type staphylococcal enterotoxin or a wild type streptococcal pyrogenic exotoxin, determined by FASTA analysis using gap penalties of −12 and −2, Blosum 50 matrix and Swiss-PROT or PIR database; or
(iii) a biologically active fusion protein comprising:
(A) said variant,
(B) said wild type staphylococcal enterotoxin,
(C) said wild type streptococcal pyrogenic exotoxin, or
(D) said fragment, operably linked to a peptide or polypeptide fusion partner

12. The method according to claim 11 wherein said peptide or polypeptide fusion partner is a tumor specific antibody, Fab fragment or single chain antibody or tumor specific ligand or receptor.

13. The method according to claim 2, wherein the toxin is a staphylococcal enterotoxin selected from the group consisting of SEA, SEB, SEC, SED, SEE, SEG, SEH, SEI, SEJ, SEK, SEL, SEM, SEN, SEO, SEP, SER, SEU.

14. The method according to claim 2, wherein said toxin is a mutant or variant of a wild type toxin which has the biological activity of the wild type toxin and has sequence homology characterized as a z value exceeding 13 when the sequence of the variant or said fragment is compared to the sequence of a wild type toxin, determined by FASTA analysis using gap penalties of −12 and −2, Blosum 50 matrix and Swiss-PROT or PIR database or a biologically active fusion protein comprising said mutant or variant fused to a peptide or polypeptide fusion partner.


SEQUENCE LISTING (12 sequences)

SEQ ID NO: 1 - DNA, 26 bp, Artificial Sequence
  EPO HRE consensus sequence
ccgggtagctggcgtacgtgctgcag

SEQ ID NO: 2 - DNA, 1712 bp, Staphylococcus aureus
gaactaggtagaaaaataattatgagaaaacactatgttgttaaagatgttttcgtatataagtttaggtgatgtatagttacttaattttaaaagcataacttaattaatataaataacatgagattattaaatataattaagtttcttttaatgtttttttaattgaatatttaagattataacatatatttaaagtgtatctagatactttttgggaatgttggataaaggagataaaaaatgtataagagattatttatttcacatgtaattttgatattcgcactgatattagttatttctacacccaacgttttagcagagagtcaaccagatcctaaaccagatgagttgcacaaatcgagtaaattcactggtttgatggaaaatatgaaagttttgtatgatgataatcatgtatcagcaataaacgttaaatctatagatcaatttctatactttgacttaatatattctattaaggacactaagttagggaattatgataatgttcgagtcgaatttaaaaacaaagatttagctgataaatacaaagataaatacgtagatgtgtttggagctaattattattatcaatgttatttttctaaaaaaacgaatgatattaattcgcatcaaactgacaaacgaaaaacttgtatgtatggtggtgtaactgagcataatggaaaccaattagataaatatagaagtattactgttcgggtatttgaagatggtaaaaatttattatcttttgacgtacaaactaataagaaaaaggtgactgctcaagaattagattacctaactcgtcactatttggtgaaaaataaaaaactctatgaatttaacaactcgccttatgaaacgggatatattaaatttatagaaaatgagaatagcttttggtatgacatgatgcctgcaccaggagataaatttgaccaatctaaatatttaatgatgtacaatgacaataaaatggttgattctaaagatgtgaagattgaagtttatcttacgacaaagaaaaagtgaaattatattttagaaaagtaaatatgaagagttagtaattaaggcaggcacttatagagtacctgccttttctaatattatttagttatagttatttttgttatatctctctgatttagcattaaccccttgttgccattatagttttcaccaactttagctgaaattgggggatcatttttatctttactatggatagttactgtgtcgccgtttttaacgatttgtttctcttttaatttgtcagttaattttttccatgcatcatttgcgtcaaacctatttccatttggatttattcttgacaaatcaattcttttaacactatcggtattaatcggcttgttattaaaattactaagttcatctaaatcagctgtacccgtaatactactttcgccaccattatttaaattgtacgtaacaccaactgtctcatttgctgttttatcgataatatttgcttctttcaaagcatctcttacatttttccataagtctctatctgttatttcagaagcctttgcaacgttattaataccattataatttgaagaagaatgaaaacctgaacctactgttgttaaaactaaagcacttgctatcaatgttcttgttaatagttttttattcattttattttctcctataacttatttgcaatcgat

SEQ ID NO: 3 - protein, 31 aa, Staphylococcus aureus
RSITVRVFEDGKNLLSFDVQTNKKKVTAQEL

SEQ ID NO: 4 - protein, 30 aa, Staphylococcus aureus
NVLIRVYENKRNTISFEVQTDKKSVTAQEL

SEQ ID NO: 5 - protein, 18 aa, Unknown
  Thosea asigna virus
EGRGSLLTCGDVEENPGP

SEQ ID NO: 6 - DNA, 35 bp, Artificial Sequence
  primer
ggcactcgagatgggtctcaacccccagctagttg

SEQ ID NO: 7 - DNA, 36 bp, Artificial Sequence
  primer
gccgtctagactacgagtaatccatttgcatgatgc

SEQ ID NO: 8 - DNA, 2056 bp, Mus musculus
cgtcttggtgggacttcagctttccagagtttatgactactgtgcctgcagcatcatggccacgtgcctgttcctcctgggccttttcctgctgctgccacgacctgtccctgctccctgctacactgccactcggtcagaatgcaagcagaagcacaagttcgtgccaggtgtatggatggctggggaaggcatggatgtgactaccctccgccgctccggctccttcccagtgaacacacagaggttcctgaggcctgaccgcacctgcaccctctgtaaaaactccctaatgagagacgccacacagcgcctacctgtggcaatcacccactggcggcctcacagctcacactgccagcgtaatgtggccgcagccaaggtccactccacggagggtgtggcccgggaggcagctgctaatatcaataacgactggcgtgtggggctggatgtgaaccctaggccagaggcaaacatgcgcgcctccgtggctggctcccactccaaggtagccaattttgcagctgagaagacctatcaggaccagtacaactttaatagcgacacagtagagtgtcgcatgtacagttttcgcctggtacaaaaacctccactccaccttgacttcaaaaaggcgctcagagccctcccccgcaactttaacagctccacagagcatgcttaccacaggctcatctcctcctatggcacgcactttatcacggctgtggacctcggtggccgcatctcggtccttacagccctgcgtacctgtcagctgaccctgaatgggctcacagctgatgaggtaggagactgcctgaacgtggaggcccaggtcagcatcggtgcccaagccagcgtctccagtgaatacaaagcttgtgaggagaagaagaaacagcacaaaatggccacctctttccaccagacctaccgtgagcgtcacgtcgaagtacttggtggccctctggactccacgcatgatctgctcttcgggaaccaagctacaccagagcagttctcaacctggacagcctcactgcccagcaaccctggtctggtggactacagcctggagcccctgcacacattactggaagaacagaacccgaagcgggaggctctgagacaggctatcagccattatataatgagcagagcccggtggcagaactgtagcaggccctgcaggtcaggccagcataagagtagccatgattcatgccagtgtgagtgccaggattcaaaggtcaccaaccaggactgctgcccacgacagaggggcttggcccatttggtggtaagcaatttccgggcagaacatctgtggggagactacaccacagctactgatgcctacctaaaggtcttctttggtggccaggagttcaggaccggtgtcgtgtggaacaataacaatccccggtggactgacaagatggactttgagaatgtgctcctgtccacagggggacccctcagggtgcaggtctgggatgccgactacggctgggatgatgaccttcttggttcttgtgacaggtctccccactctggtttccatgaggtgacatgtgagctaaaccacggcagggtgaaattctcctaccatgccaagtgtctgccccatctcactggagggacctgcctggagtatgccccccaggggcttctgggagatcctccaggaaaccgcagtggggctgtgtggtaacataataacaacaataacatgcctgagagctgggtgtagtagcacacgcctttaatcccagcatttgggaggcagagacaggtggatatctatgagttcgaggccagcctgggtctacagggtctcaaaaaaaaaaaaagcaaacaacaaaactggaatgttcaactggcttctccctggggatctgcaatggcttactatgcatagagaggccactagagtggctgagtttttacaatagagcatccctgactttcccttccacactgcctcagccctgcaatgcccgaaagcttggcaactactgccacgaagcgtaaacatgggccaggagcc

SEQ ID NO: 9 - DNA, 1418 bp, Mus musculus
agagggggtacaaggtcacagagccccctctgccttcttcctctcctagaggttaaaagagagcaaggacaacactcttgacgctgggacctaggcggccttccggggaagatgaagatcctcctgctactgctgaccttgtctctggcctccaggacaaaggcaggggagatcatcgggggacatgaagtcaagccccactctcgaccctacatggccttactttcgatcaaggatcagcagcctgaggcgatatgtgggggcttccttattcgagaggactttgtgctgactgctgctcactgtgaaggaagtataataaatgtcactttgggggcccacaacatcaaagaacaggagaagacccagcaagtcatccctatggtaaaatgcattccccacccagactataatcctaagacattctccaatgacatcatgctgctaaagctgaagagtaaggccaagaggactagagctgtgaggcccctcaacctgcccaggcgcaatgtcaatgtgaagccaggagatgtgtgctatgtggctggttggggaaggatggccccaatgggcaaatactcaaacacgctacaagaggttgagctgacagtacagaaggatcgggagtgtgagtcctactttaaaaatcgttacaacaaaaccaatcagatatgtgcgggggacccaaagaccaaacgtgcttcctttcggggggattctggaggcccgcttgtgtgtaaaaaagtggctgcaggcatagtttcctatggatataaggatggttcacctccacgtgctttcaccaaagtctcgagtttcttatcctggataaagaaaacaatgaaaagcagctaactacagaagcaacatggatcctgctctgattacccatcgtccctagagctgagtccaggattgctctaggacaggtggcaggatctgaataaaggactgcaaagactggcttcatgtccattcacaaggaccagctctgtccttggcaggccaatggaacacctcttctgccaccatgctgtgacaacccaactgacatcttcctatggaagtttgccctctccacaaaagaagtagaatgtttgcattggagctgggcatgctctgcttcccctcagtgccccgagaatgttatctaatgctagtcatcattaatagctccctacagaactttcatacagttgcacccaagttgctgatgtgttctctagaatagagcaagaaatagtaaacagaattccttttgcctctctgtactattttcccccaaataccaagatttgtatgttttataaagctaatttccttatcaaatgacatcttttaatttttacattaatggcttattttcaaggtacaacctgatttttttatggacaaaaatgatgtaaaatcaaataaaa

SEQ ID NO: 10 - DNA, 1591 bp, Mus musculus
ggcactgcaaggctgcttctcaggcattcaggctgcggctgcaggcaccgcgagcccggagcacccacgagctgagtgtgcaggacgcaccccagcacagccacctacggccgctgaatgaagcttccaggagtccgcccccggccggctgcgccccgtcggaggtgcacccgctgagagcgcctgggcaccgaaagccggtgcgctcacctgctaacctgccagccatggggccacacgggaacgacagcgacttcttgctggcacccaacggaagccgagcgccagaccacgacgtcactcaggaacgggacgaagcgtgggttgtgggcatggccatcctcatgtcggttatcgtcctggccatcgtgtttggcaacgtgctggtcatcacagccattgccaagttcgagcgactacaaaccgtcaccaactacttcataatctccttggcgtgtgctgatctagtcatgggcctagcggtggtgccgtttggggccagtcacatccttatgaaaatgtggaattttggcaacttctggtgcgagttctggacttccattgatgtgttgtgcgtcacagccagcatcgagaccctgtgcgtgattgcagtggatcgctatgttgctatcacatcgcccttcaagtaccagagcctgctgaccaagaataaggcccgagtggtcatcctgatggtatggattgtatctggccttacctcctttttgcctatccagatgcactggtaccgtgccacccacaagaaagctatcgattgttacaccgaggagacttgctgtgacttcttcacgaaccaggcctacgccatcgcgtcctcgattgtgtctttctacgtgcccctggtggtgatggtctttgtctattcccgggtcttccaggtggccaaaaggcagctgcagaagatagacaaatctgaaggaagattccacgcccaaaacctcagccaggtggagcaggatgggcggagcggccacggactccgaaggtcctccaagttctgcttgaaagagcacaaagccctcaagactttaggcatcatcatgggcacattcaccctctgctggctgcccttcttcattgtcaatatcgtgcacgttatcagggacaacctcatccctaaggaagtttacatcctccttaactggttgggctacgtcaactctgccttcaatcctcttatctactgtcggagtccagatttcaggattgcctttcaagagcttctgtgccttcgcaggtcttcttcgaaaacctatgggaacggctactctagcaatagcaacggcagaacggactacacaggggagccaaacacttgtcagctggggcaggagagagaacaggaactgctgtgtgaggatcccccaggcatggaaggctttgtgaactgtcaaggtactgtgcctagccttagcgttgactcccaaggaaggaactgtagtacaaatgactcgccactgtaatacaggctttctactctctaagacccctccttgacaggacactaaccagactatttaacttgagtgtaataactttagaataaaattgtatagagatttgcagaagg

SEQ ID NO: 11 - protein, 9 aa, Unknown
  CD63 sorting signal
SIRSGYEVM

SEQ ID NO: 12 - protein, 10 aa, Pseudomonas aeruginosa
KKKVTAQELD